SEQUENCE LISTING (2 sequences)

SEQ ID NO: 1 - DNA, 27 bp, Artificial Sequence
  Description of Artificial Sequence Synthetic primer
gatcaccggttcaaaaacaatcttaca

SEQ ID NO: 2 - DNA, 21 bp, Artificial Sequence
  Description of Artificial Sequence Synthetic primer
agcggctgaaggcaaatatca

(19) United States
(12) Patent Application Publication
(10) Pub. No.: US 2012/0077260 A1
(43) Pub. Date: Mar. 29, 2012

(54) RESERVOIR-BUFFERED MIXERS AND REMOTE VALVE SWITCHING FOR MICROFLUIDIC DEVICES

(75) Inventors: Andre Sharon, Newton, MA (US); David A. Chargin, Somerville, MA (US); Paul Mirsky, Jamaica Plain, MA (US); Alexis Sauer-Budge, Lincoln, MA (US)

(73) Assignees: Fraunhofer USA, Inc., plymouth, MI (US); Trustees of Boston University, Boston, MA (US)

(21) Appl. No.: 13/262,570

(22) PCT Filed: Mar. 30, 2010
(86) PCT No.: PCT/US10/29272

§ 371 Date: Dec. 1, 2011

Related U.S. Application Data
(60) Provisional application No. 61/164,756, filed on Mar. 30, 2009.

(51) Int. Cl. C12M 1/40; G01N 21/75; B01L 3/00

(52) U.S. Cl. 435/287.2; 422/502; 435/289.1; 422/82.05

(57) ABSTRACT

The present invention relates generally to the control of fluid flow rate and direction on a microfluidic device. In particular, the present invention provides an integrated valveless microfluidic device, where directional fluid control is controlled using off-chip remote valve switching and fluid flow rate changes are controlled using on-chip flow-rate changing fluid reservoirs. The present invention provides methods and systems for directional fluid control and control of fluid flow rate in an integrated microfluidic device which enables processes with different flow rates to be performed on one device without the need of on-chip valves.

### CROSS REFERENCE TO RELATED APPLICATIONS

This application claims priority under 35 U.S.C. 119(e) of U.S. Provisional Patent Application Ser. No. 61/164,756 filed Mar. 30, 2009, the contents of which are incorporated herein by reference in their entirety.

### FIELD OF THE INVENTION

The present invention generally relates to the field of microfluidic devices, and in particular integrated microfluidic devices and the control of the movement of fluid within microfluidic devices. In particular, the present invention relates to methods, devices, systems and instruments for directional fluid control and rate of fluid control, using remote valve switching devices and systems and reservoir buffered devices and systems respectively.

### BACKGROUND OF THE INVENTION

Currently, the majority of infection diagnoses, e.g. bacterial, parasites, fungi, viruses are conducted via cultures² or immunoassays or PCR (e.g. viruses), which can take many hours to days. Thus, physicians will typically prescribe an initial broad-spectrum drug therapy at the initial examination and then change the therapy as needed upon receipt of the culture results. This practice contributes to the rise of antibiotic resistance and is often ineffective at treating the patient. Moreover, some infectious agents are difficult to culture with standard laboratory procedures, contributing to the number of unidentified cases. To address these concerns, rapid diagnostics have been developed; both immunoassays and nucleic acid based tests (NAT). The immunoassays often suffer from inadequate sensitivity and/or specificity³, while NATs are expensive⁴. NATs require specialized lab space, expensive reagents, and extensively trained technicians to appropriately conduct the assays. In practice, these factors lead to batch processing once a day in most clinical labs, making the effective turn-around-time 24 hours⁵. To address these challenges and provide a truly rapid test that meets the demands of high sensitivity and specificity, many have proposed a point-of-care molecular diagnostic that automates the sample preparation, nucleic acid amplification, and detection in a miniaturized lab-on-a-chip format⁶⁻⁸. However, much of the work in this field has yet to result in a fully integrated lab-on-a-chip or a design that is truly low cost to manufacture, or low cost integrated lab-on-a-chip which can control fluid flow rate and/or velocity and direction, or enable multiple manipulations on one chip.

Only two examples of fully integrated microfluidic chips for nucleic acid based analysis with sample in-answer out capability have been reported to date^{9, 10}. Both examples are impressive in the level of integration and functionality, but are limited in utility in that they are complicated and expensive to manufacture due to the multiple material types, the multi-level structures, and the number of assembly steps required. Thus, there exists a need in the art for a low-cost automated lab-on-a-chip device for point-of-care molecular diagnostic truly rapid test that meets the demands of high sensitivity and specificity.

### SUMMARY OF THE INVENTION

Sample preparation and analysis, e.g. nucleic acid based tests (NATs) and immunodetection on microfluidic devices typically requires processing the sample through different multiple microfluidic devices in a sequential manner and transferring the sample from one microfluidic device to another. Even where the microfluidic devices are fluidly connected to each other, the transfer of the sample between the microfluidic devices can increase risk of sample contamination and loss of sample volume. Additionally, as each microfluidic device serves a different function, the velocity of the flow of fluid through each the microfluidic device needs to controlled for optimal functioning of each device. For example, the velocity of a sample through a microfluidic device for cell lysis or sample mixing is different (e.g. at a faster velocity) than the velocity of a sample through a microfluidic device configured for, e.g. nucleic acid extraction, which requires a slow velocity. Accordingly, control of sample velocity needs to be carefully controlled if a sample is sequentially processed through different microfluidic devices, each requiring a different sample velocity.

The control of fluid velocity between microfluidic devices is typically controlled by on-chip valves which regulate and control the fluid flow between each microfluidic device. Similarly, on-chip valves are used between modules that serve different functions in single microfluidic device. However, existing microfluidic devices and lab-on-a-chip which comprise multiple modules which require different velocities are highly complicated devices, often comprising on-chip valves, multiple layers and multi-level structures, multiple material types and a number of assembly steps required. The presence of on-chip valves and multiple layers on microfluidic chips results in very expensive manufacturing costs not suitable for a cheap-disposable lab-on-a-chip system.

Herein, the inventors demonstrate use of a simple, disposable integrated microfluidic device which comprises reservoirs to serve as fluid buffers to control and change the rate and/or velocity of fluid flow through the device, thus enabling both high-flow processes and low-flow process to be present on the same microfluidic chip, and where the direction of the fluid is controlled by off-chip, remote switching valves. As the microfluidic chip does not comprise any on-chip valves, it can be produced at very low manufacturing costs, yet maintaining both high-flow and low-flow components on the same microfluidic device, thus reducing sample transfer issues (e.g. contamination and sample loss) and enabling a fully integrated microfluidic chip for high flow and low flow processes on a single chip which is disposable.

Additionally, the remove valve switching mechanism which interfaces with the microfluidic device can be used with any generic microfluidic chip which is configured to use the same interface. In some embodiments, a generic microfluidic chip used with the remote valve switching has at least one reservoir to buffer changes in fluid flow as disclosed herein. In alternative embodiments, the generic microfluidic chip used with the remote valve switching does not have a reservoir. As such, the present invention provides a dynamic and diverse system in which fluid flow on the microfluidic chip can be controlled remotely, and in embodiments where the microfluidic chip comprises reservoirs to buffer the rate and/or velocity of fluid flow from a high flow and a low flow process, and vice versa, the microfluidic chip can process the sample through multiple manipulations on one chip, such that the chip can be tailored and selected for a specific function or assay (e.g. nucleic acid assay, PCR assay, immunoassay etc.).

Accordingly, one the benefits of the remote-valve switching and the presence of reservoirs on the microfluidic chips as disclosed herein enables a dynamic microfluidic system in a format which can be tailored to the specific needs of the assay. The remote valves used to control fluid direction and velocity are controlled by a control unit can be automated to provide both improved reproducibility of sample preparation and fast sample analysis, allowing for quick sample turn-around. Furthermore, the presence of reservoirs on the microfluidic chips provides a format enabling multiple manipulations to be integrated onto a single microfluidic chip without complicated multi-layer formats or presence of on-chip valves. This is highly advantageous for reduced cost of manufacturer of the integrated microfluidic device, particularly suitable for production of single-use, disposable microfluidic chips for point-of-care sample analysis.

Herein, the inventors have demonstrated a fully functional microfluidic system, as shown in FIGS. 1 and 2A-2B, which comprises a control unit for automated remote control of the fluid flow in the microfluidic chip, and a sample analysis or detection subsystem for end-sample analysis, such as an optical detection subsystem. In some embodiments, the optical detection subsystem is connected to a computer and also an optical interface which interfaces with part of the microfluidic chip to analyze and detect the presence of a labeled end-sample, e.g. a fluorescent sample present in a detection well on the microfluidic chip. The optical interface which can analyze the presence of fluorescence on a sample is advantageous for continuous-sample analysis, for example real-time sample analysis. In alternative embodiments, the optical detection subsystem can analyze an end-sample off the chip. In some embodiments, microfluidic system further optionally comprises a thermal interface which is controlled by a thermal controller for integrated temperature cycling on the microfluidic device.

Importantly, the interface of the fluid control system has the capability of accommodating various chip different designs, including any generic microfluidic chip, as well as microfluidic chips comprising reservoir buffers which allow sample processing through both high and low flow processes on a single integrated microfluidic chip. In some embodiments, a generic microfluidic chip used with the remote valve switching has at least one reservoir to buffer changes in fluid flow as disclosed herein. In alternative embodiments, the generic microfluidic chip used with the remote valve switching does not have a reservoir.

Accordingly, the present invention generally relates to a microfluidic system comprising a fully integrated microfluidic chip, where directional fluid flow is controlled off-chip using remote-valve switching, where the valves are controlled by a control unit, e.g. a fluid control subsystem, and where rate and/or velocity of fluid flow, e.g. changes in rate of fluid flow/velocity, is controlled using an on-chip reservoir-buffers present on the microfluidic device. In some embodiments, the integrated microfluidic chip comprising at least one flow changing reservoir can be used by its self (e.g. independent of the system described herein), or alternative embodiments, with the fluid control subsystem using the remote valves as disclosed herein.

Stated another way, the present invention relates to a system where a change in direction of fluid flow on a microfluidic chip is controlled off-chip using a remote valve switching system, and a change in velocity of fluid flow on the microfluidic chip is controlled on-chip using reservoir buffers. This is a highly diversifiable system, enabling fluid flow through a disposable integrated microfluidic chip, (e.g. a microfluidic chip comprising components requiring both high- and low velocities for different processes to be performed on a single chip) to be controlled in an automated manner, without the need of expensive on-chip valves. As the disposable integrated microfluidic chip is valve-less, it can be produced at easily and rapidly with low manufacturing costs.

One aspect of the present invention therefore relates to a remote valve switching system, connected to a control unit for directional fluid control of fluid on a microfluidic device which lacks valves or pumps on the microfluidic chip. Accordingly, one aspect of the invention relates to a remote-valve microfluidic system.

Another aspect of the present invention relates to control of velocity and/or rate of fluid flow, and in particular to allowing velocity and flow rate changes on a valveless microfluidic device. According to this aspect of the invention, the inventors incorporated flow-rate and/or velocity changing fluid-reservoirs, herein referred to as “reservoir-mixer buffers” or “reservoirs” to increase or decrease the fluid velocity and/or flow rate on a microfluidic device. Accordingly, one aspect of the invention relates to a microfluidic device comprising at least one reservoir to change (i.e. increase or decrease) the velocity and/or flow rate on the fluid on the microfluidic chip. In some embodiments, the reservoirs can be used to change the velocity and/or flow rate from high to low, or alternatively, to change the velocity and/or flow rate from low to high. As such, the reservoir comprising microfluidic chips enable samples to be processed through multiple components on a single integrated chip which required different velocities and/or flow rates, without the need of expensive on-chip valves and the like.

In one embodiment, a reservoir is located on a microfluidic device between at least two chambers requiring different velocities. For example, as shown FIGS. 3A and 3B, where it is desirable to use a reservoir to decrease velocity on a microfluidic device, a reservoir is fluidly connected to and located between a high flow chamber (e.g. a mixer) and a high flow chamber (e.g. a nucleic acid extraction column), where the input of the reservoir receives a fluid stream from the output of a high flow chamber, and the reservoir output is fluidly connected to the input of a low flow chamber.

In one embodiment, as shown in FIGS. 4A and 4B, where it is desirable to use a reservoir to increase the velocity on a microfluidic device, a reservoir is fluidly connected to and located between a high flow chamber (e.g. a nucleic acid extraction column) and a high flow chamber (e.g. a mixer), where the input of the reservoir receives a fluid stream from the output of a low flow chamber, and the reservoir output is fluidly connected to the input of a high flow chamber.

As such, one aspect of the present invention relates to a microfluidic device comprising at least one reservoir, where change of direction of the fluid flow on the microfluidic device is controlled using remote-valve switching as disclosed herein, and change of rate and/or velocity of fluid flow on the microfluidic device is controlled using reservoir-buffered chambers as disclosed herein. FIGS. 3C and 4B show flow diagrams of embodiments of a method and system to control change in fluid direction (using at least two remote valves), and change in fluid velocity (using a reservoir buffers) through a integrated microfluidic chip, where FIG. 3C shows an embodiment of the steps for configuring the remote-valves open/closed order to direct the fluid into the reservoir to change the velocity from a high to a low velocity, and FIG. 4B shows an embodiment of the steps to configure the remote-valve open/closed order to direct the fluid into the reservoir to change the velocity from a low to a high velocity.

One advantage of the microfluidic chips comprising flow-rate and/or velocity changing fluid reservoirs is that they have a very low-cost of manufacture due to lack of on-chip valves, and also enables multiple processes using different flow velocities to be performed on a single microfluidic chip. Thus, microfluidic chips comprising and/or velocity changing fluid reservoirs are particularly suited for use as single-use, fully-integrated disposable chips for complete sample processing and analysis in a high throughput manner.

In particular, in order to achieve a truly low-cost integrated disposable microfluidic chips, in which fluid flow can be regulated in an automated manner, the inventors have minimized the cost of the disposable component by removing all active components from the chip, enabling the chip to have a planar design and to be manufactured using low-cost methods such as injection molding, where a minimal number of assembly steps are required. Additionally, the inventors have used material with dimensional stability at high temperatures (unlike PDMS) and that could be manufactured reproducibly without caustic chemicals (such as hydrofluoric acid which is used to etch glass). In alternative embodiments, one can make the disposable microfluidic chip from any suitable material known by persons of ordinary skill in the art, for example, but not limited to plastics (e.g. acrylic, polycarbonate, etc) and other cheap, durable stable synthetic materials where thermal stability and/or on-chip fluorescence detection is not important.

As disclosed herein, the inventors designed a plastic microfluidic chip in a planar format without any active components, which is amenable to injection molding and utilizes a novel porous polymer monolith (PPM) embedded with silica or carbon nanoparticles which the inventors have previously demonstrated has been shown to lyse bacteria and isolate the nucleic acids from clinical samples¹. In some embodiments, the microfluidic chip is made of ZEONEX® (ZEONEX 690R), a thermoplastic with a high melting temperature to allow PCR, good UV transmissibility for UV-curing of the PPM, and low auto-fluorescence for fluorescence detection and analysis of the PCR product amplicon.

In some embodiments, the present invention relates to any integrated microfluidic device comprising a and/or velocity changing reservoir which is configured to process or lab-on-a-chip function, for example, microfluidic devices for the detection of nucleic acids in a solution, e.g. bacteria in a liquid sample or sample suspension, as disclosed in the Examples. In one embodiment, as shown in FIGS. 5A and 5B, a microfluidic device comprising a flow velocity changing reservoir comprises at least one or more components to conduct bacterial lysis, nucleic acid isolation (e.g. a solid phase extraction (SPE) column), nucleic acid concentration and polymerase chain reaction (PCR) (e.g. a PCR channel). The PCR product can be analyzed using fluorescent detection using the sample analysis detection subsystem as shown in FIGS. 1 and 2A. In particular, in addition to demonstrating a proof of principal of detection of nucleic acid from bacteria (see Example 2), the inventors have demonstrated nucleic acid extraction from a variety of samples, e.g., virus, bacteria, mammalian cells, and includes nucleic acid such as DNA, RNA, and the like. In particular, the inventors have previously established that the same microfluidic chip shown in FIG. 5A can be used for detection of influenza RNA from nasopharyngeal washes and transrenal DNA (data not shown).

In some embodiment, a microfluidic device can comprise multiple reservoir-buffer chambers to enable multiple changes in velocities of the same chip, as shown in the Examples and in FIGS. 5A and 5B. In other words, a reservoir-buffer chamber serves the function to “buffer” the fluid velocity from the mixer (e.g. a high flow chamber) and a low-velocity chamber, or vice versa. In some embodiment, a high flow chamber is a mixer chamber, and a low flow chamber is a separation column such as a SPE, or porous polymer monolith (PPM) embedded with silica particles (e.g. PPM) column. In some embodiments, the reservoir can be used as an individual modular microfluidic device which can be combined with any microfluidic device known by a skilled artisan so varying velocities and/or flow rates can be used in the same microfluidic system comprising individual microfluidic devices requiring different velocities and/or flow rates. Thus, the inventors have developed a method to enable multiple microfluidic devices to be used sequentially regardless of the required velocity and/or flow rate so that any microfluidic device can be combined for a desired application.

In some embodiments, a microfluidic device comprising at least one velocity-changing reservoir can be adapted or integrated to comprise components for cell lysis, such as those designed to lyse samples, as disclosed in International Patent Application WO2009/002580 (herein incorporated in its entirety by reference), and those designed for nucleic acid separation and detection as disclosed in U.S. patent application 2007/0015179, herein incorporated in its entirety by reference. In alternative embodiments, the microfluidic device disclosed herein comprising reservoirs to buffer fluid flow between high flow processes and low flow process can be fluidly connected to other microfluidic devices, such as those disclosed in WO2009/002580 and 2007/0015179.

In some embodiments, a microfluidic device comprising at least one flow changing reservoir can further include a porous polymer monolith (PPM) impregnated with silica particles (e.g. for nucleic acid extraction) or carbon nanoparticles (e.g. for cell lysis) of a biological sample, as disclosed in WO2009/002580 or 2007/0015179, where the PPM impregnated with silica or carbon particles can be prepared via UV initiated polymerization of a porous polymer solution embedded with the silica or carbon nanoparticles, within the channel. In alternative embodiments, the microfluidic device comprising at least one flow-rate and/or velocity changing reservoir can further include a micro-column, for example micro-columns commonly known by one of ordinary skill in the art, for example, a microaffinity column packed with beads, or glass wool or fiber material, for sample preparation and isolation.

In some embodiments, as shown FIGS. 5A and 5B, a microfluidic device comprising at least one reservoir can further comprise a PCR thermal cycling component, e.g. a PCR channels where PCR thermal cycling can be achieved by any means, such as an thermal interface which contacts part of the microfluidic chip comprising the thermal cycling component, where the thermal interface comprises a PCR thermal cycling device with a ceramic heater and air cooling. In such an embodiment, analysis of the PCR product can be performed by fluorescence detection, which can be accomplished with an optical spectrometer to detect the product either on- or off the microchip. In some embodiments, as shown in FIG. 1, a thermal interface is controlled by the thermal controller connected to the control unit. In some embodiments, as shown in FIG. 1, end-sample analysis is performed by a sample analysis or sample detection subsystem. In some embodiments, where the end-sample is fluorescently labeled, the detection subsystem is an optic detection system which comprises a spectrometer and optical detection system. In some embodiments, the detection subsystem is connected to a computer. In some embodiments, as discussed earlier, the detection subsystem analyses a product which is present on the chip. In alternative embodiments, the detection subsystem analyses a product which is removed from the microfluidic devices.

Another aspect of the present invention relates to a system and apparatus as shown in FIG. 1 comprising any combination of, or all of, the following components: a control unit (e.g. a fluid control unit) connected to remote-valves, an interface connecting the remote valves with the microfluidic chip, a microfluidic chip comprising reservoirs, and a sample analysis/detection system (e.g. optical detection subsystem). In some embodiments, the system can comprise multiple sample analysis/detection systems, e.g. for optical detection and another for a different types of detection, for example, colorimetric detection, or for continuous fluorescence detection. One of ordinary skill in the art can connect the appropriate sample analysis detection systems for the appropriate microfluidic device and assay being performed. In embodiments, the system further optionally comprises a thermal interface controlled by a thermal controller which is connected to the control unit, which can be used for thermal cycling e.g. for use with a microfluidic chip configures to comprise a PCR channel. In alternative embodiments, the thermal interface controlled by a thermal controller can be used for thermal control of the microfluidic chip, e.g. isothermal amplification or for multiple heaters for continuous flow amplification.

The inventors demonstrate the integrated functionality of control of fluid direction using remote valves of an integrated microfluidic chip comprising velocity changing reservoirs using Bacillus subtilis as a model bacterial target. In one embodiment, and as proof of principle, the inventors demonstrate use of the system of FIGS. 1 and 2 for remote valve switching to control fluid flow in an integrated microfluidic chip of FIG. 5A. The chip of FIG. 5A comprises at least two reservoirs, at least two mixers, a nucleic acid extraction column (SPE column) and a PCR column and was used for integrated gene expression analysis on-chip using a TaqMan assay to detect the isolated bacterial DNA.

In particular, the microfluidic chip of FIG. 5A comprises a system for changing velocity from high- to low flow (e.g. similar to the configuration of FIG. 3), where a reservoir is located between a high flow chamber (e.g. mixer) and a low flow chamber (e.g. a SPE column), and use of the remote valve system was used to control the direction of fluid on the microfluidic chip according to the steps outlined in FIG. 3. The microfluidic chip of FIG. 5A additionally comprises a system for changing the velocity from a low- to high velocity (e.g. similar to the configuration of FIG. 4A), where a reservoir is located between a low flow chamber (e.g. a SPE column) and a high flow chamber (e.g. mixer), prior to the fluid flowing into PCR channel, and use of the remote valve system used to control the direction of fluid on the microfluidic chip according to the steps outlined in FIG. 3.

Accordingly, the inventors in Example 2 demonstrate the functionality of using remote valve switching to change fluid direction and reservoirs on the microfluidic chip to change fluid flow (from high- to low velocities; and low- to high velocities) for a fully integrated microchip system.

### BRIEF DESCRIPTION OF THE FIGURES

This patent or application file contains at least one drawing executed in color. Copies of this patent or patent application publication with color drawing(s) will be provided by the Office upon request and payment of the necessary fee.

FIG. 1 shows a schematic drawing of one embodiment of a system for remote-valve switching to control directional fluid flow on a microfluidic chip. The valves of the remote valve-switching system are controlled by a control unit, e.g. a fluid control subsystem, and where each valve is fluidly connected to an input and/or output port at the interface of an integrated microfluidic chip, where the integrated microfluidic chip comprises at least one flow changing reservoir to change (e.g. increase/decrease) the flow rate and/or velocity between a high flow and low flow process (or vice versa). In one embodiment, the control unit is connected to a computer, where the control unit is connected to, and controls the opening and closing of each valve which is fluidly connected to an input and/or output port at the interface with the microfluidic device.

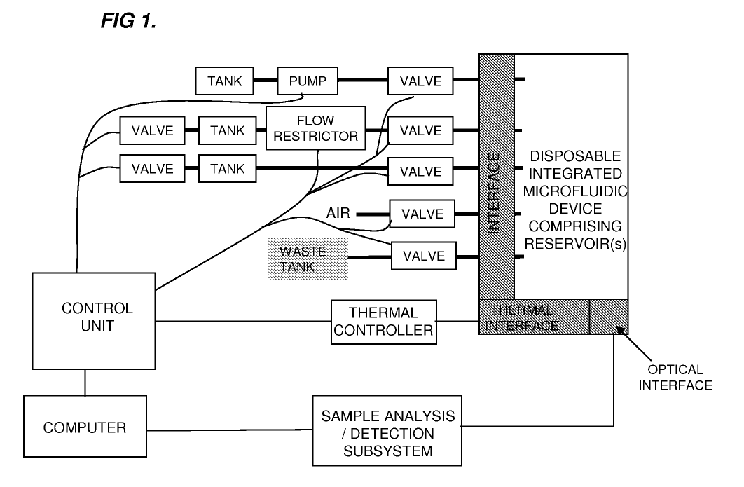

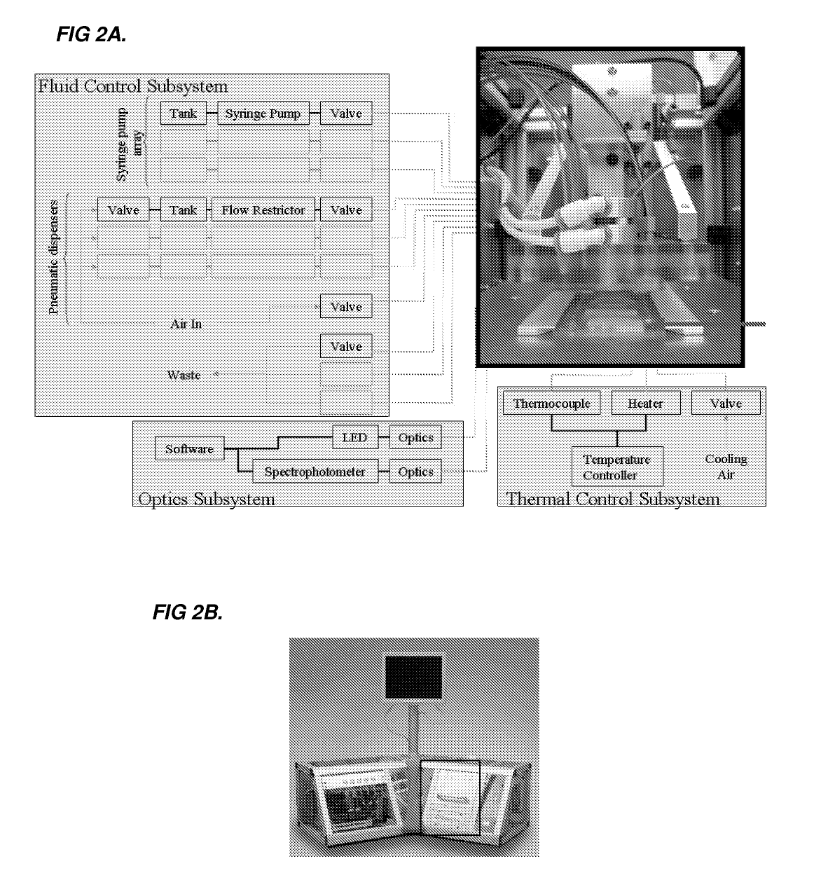

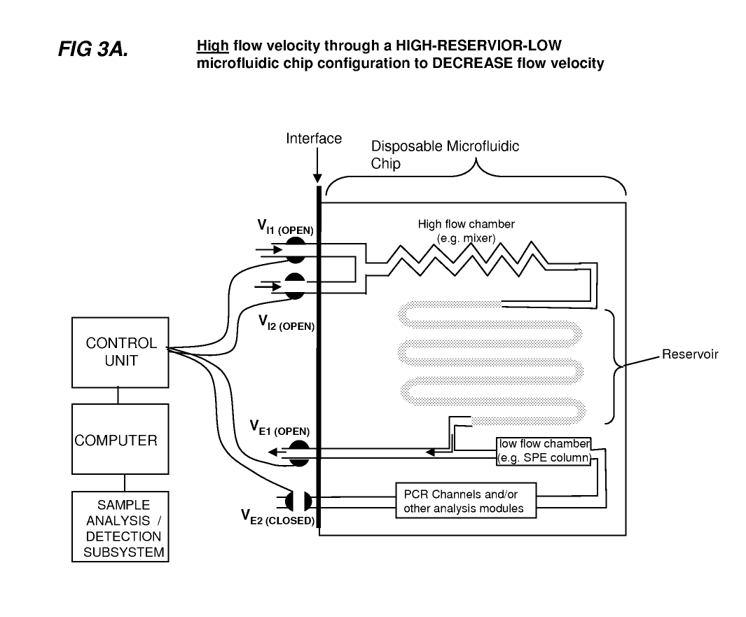

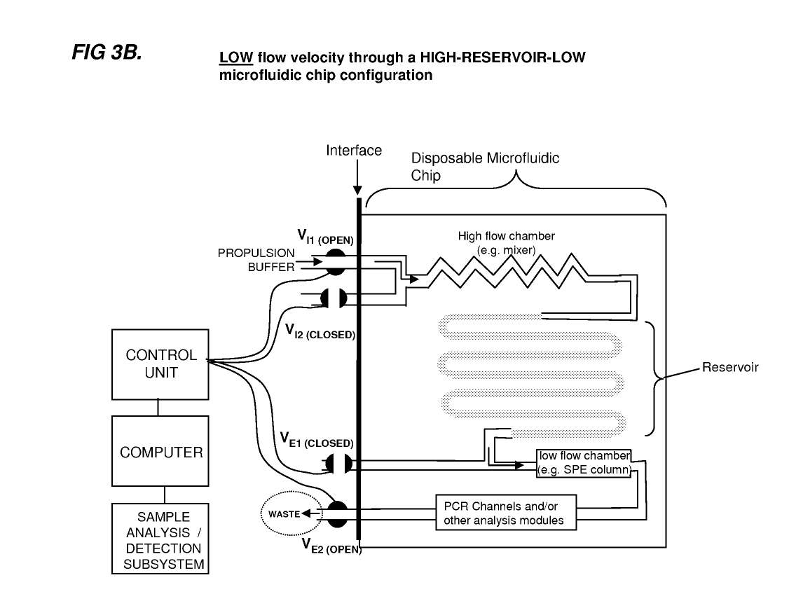

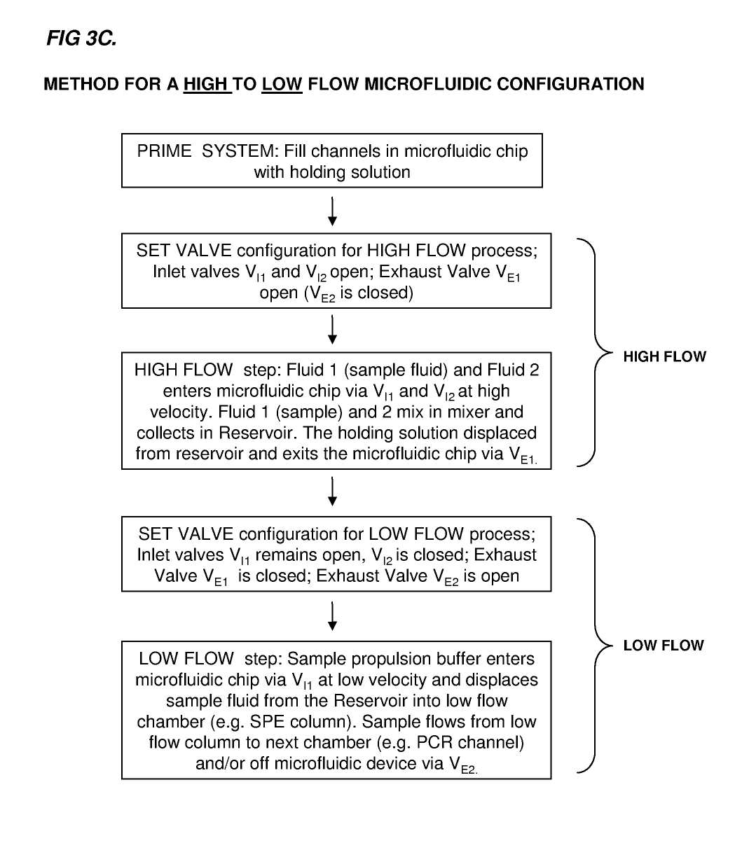

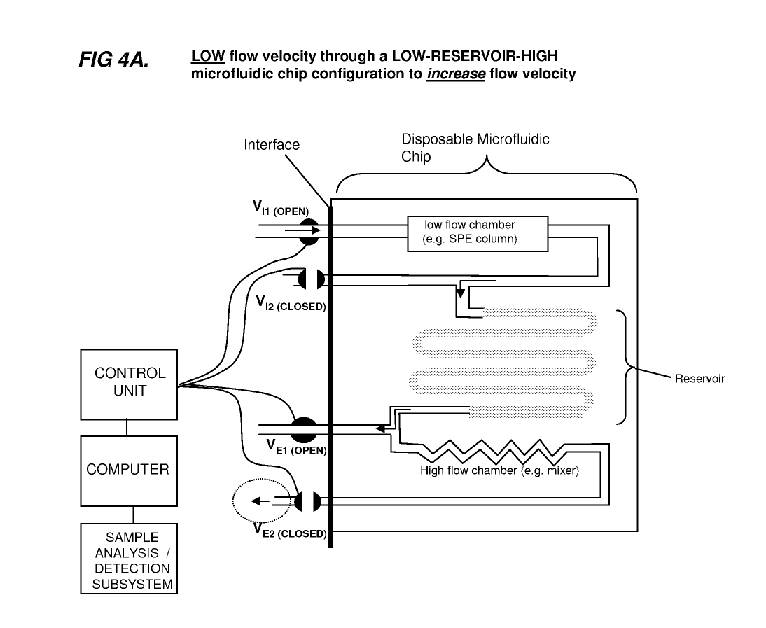

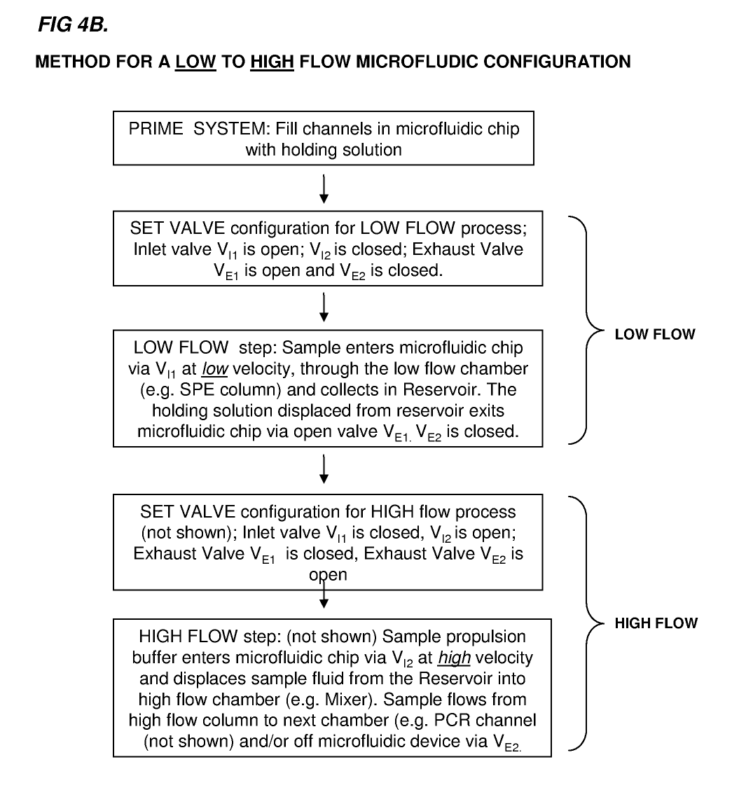

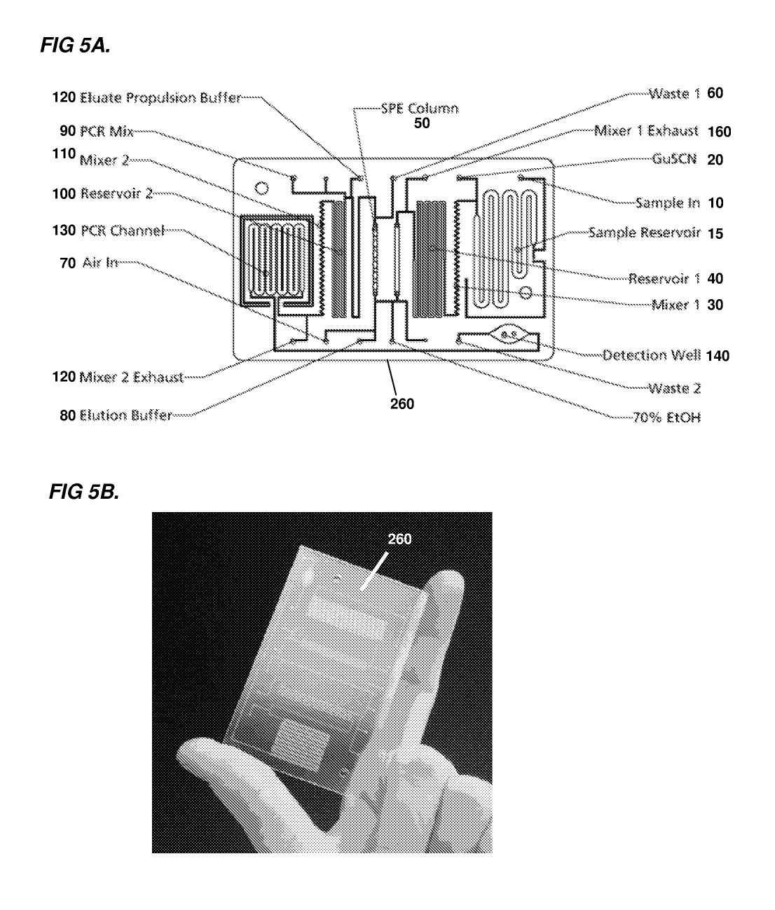

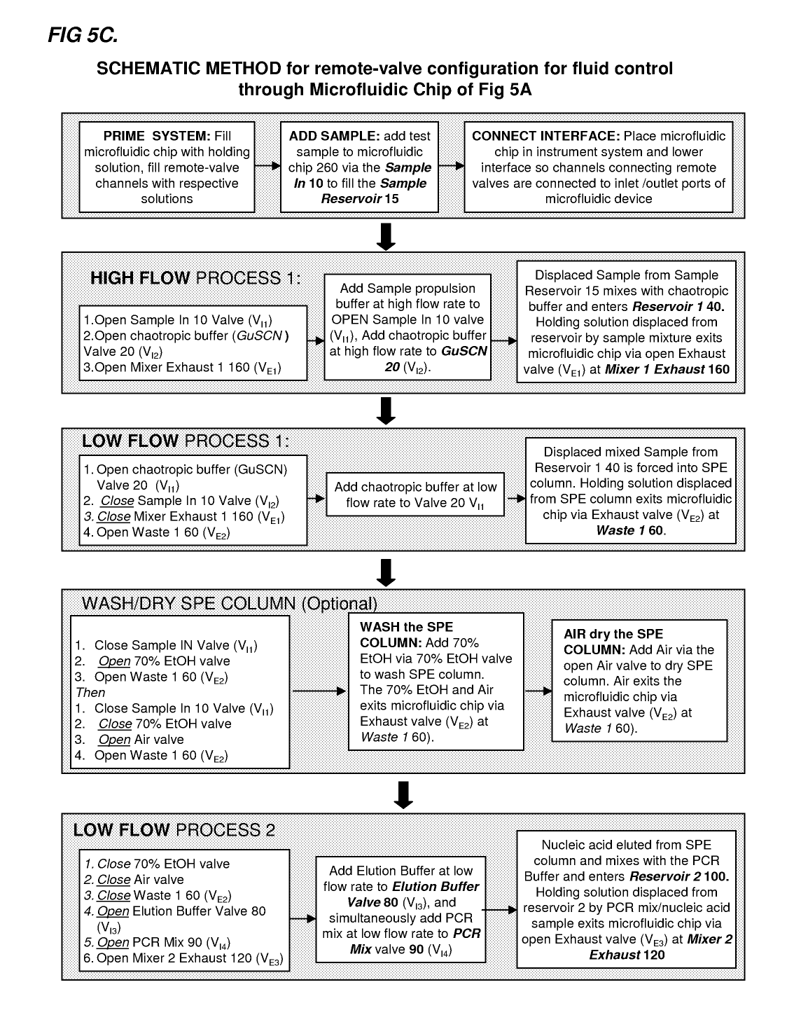

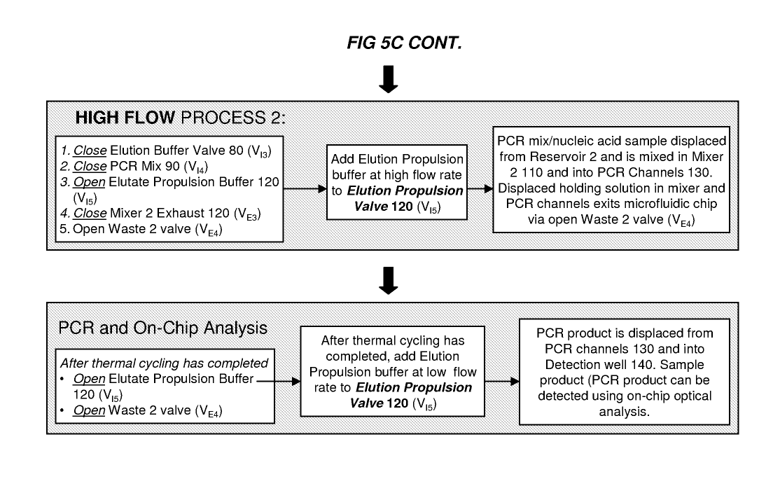

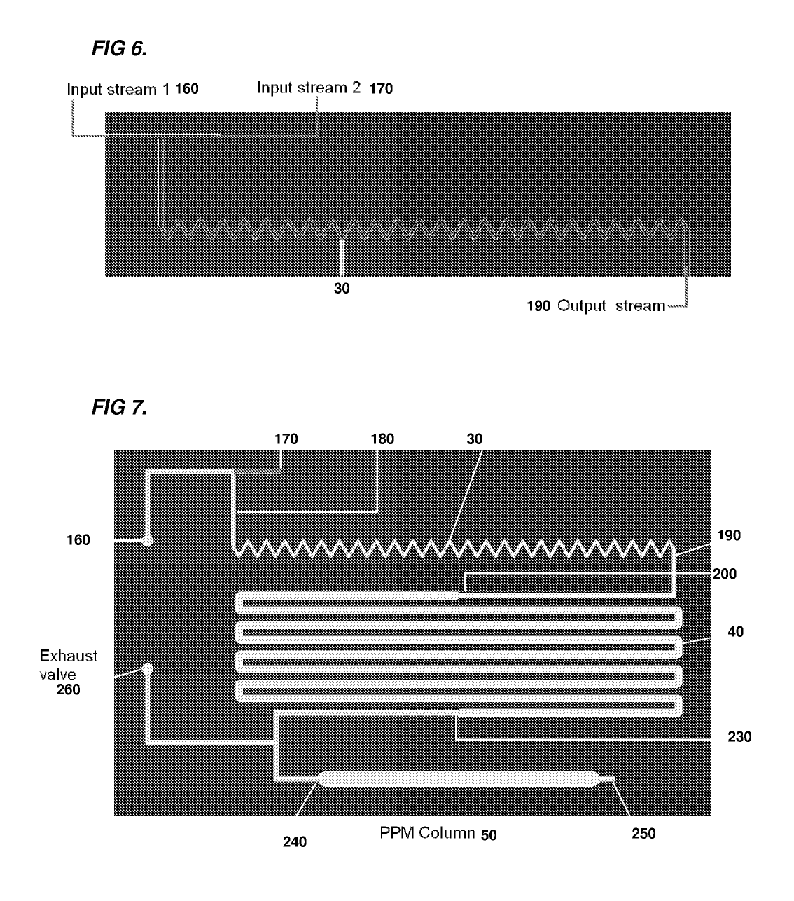

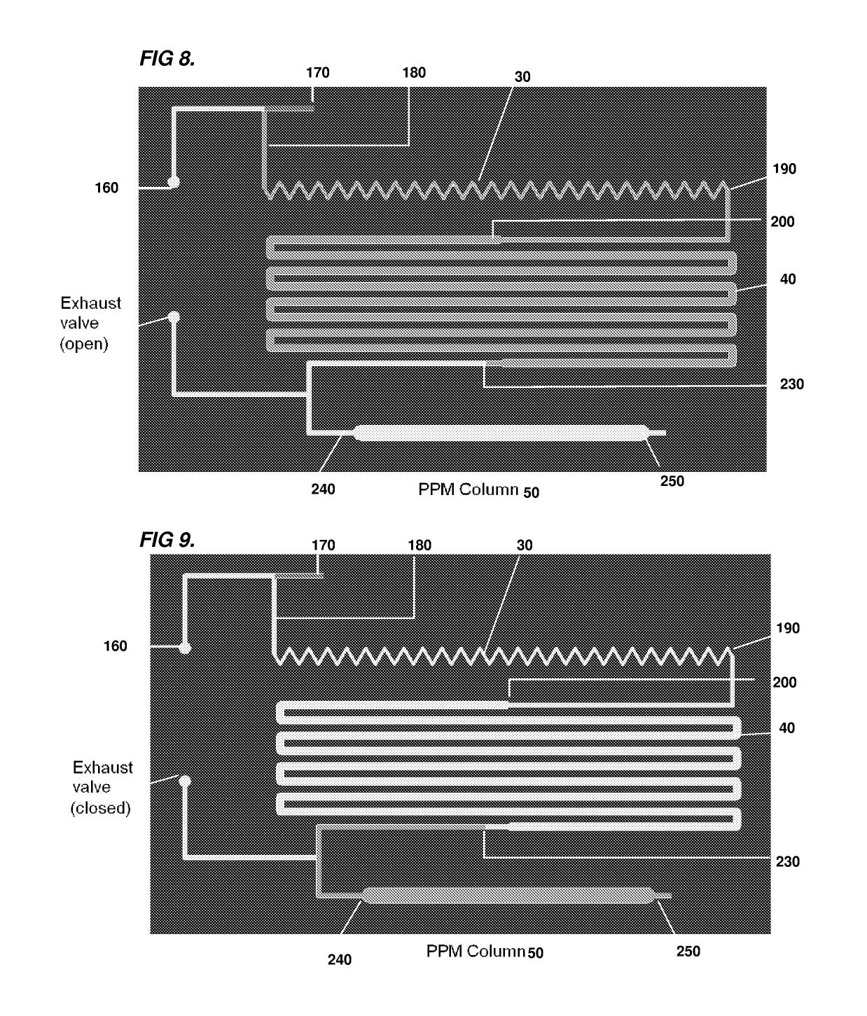

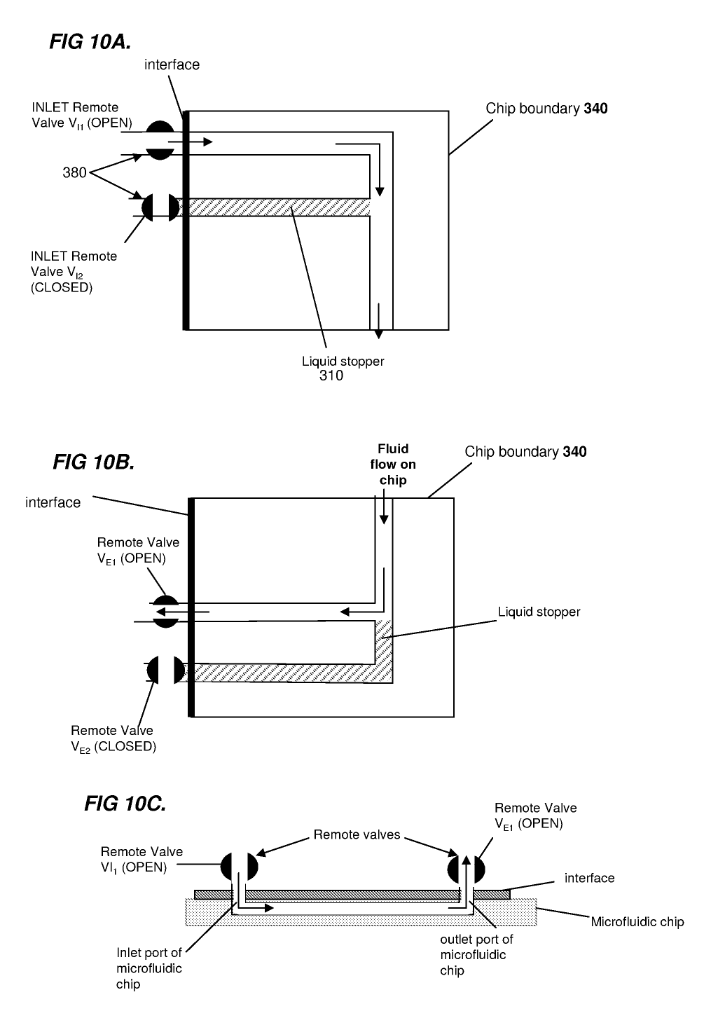

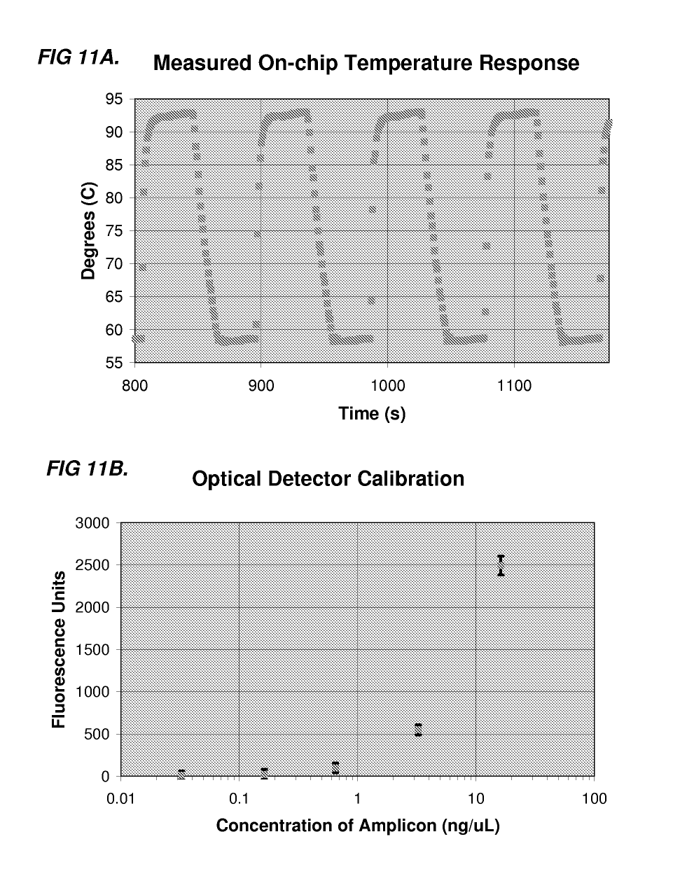

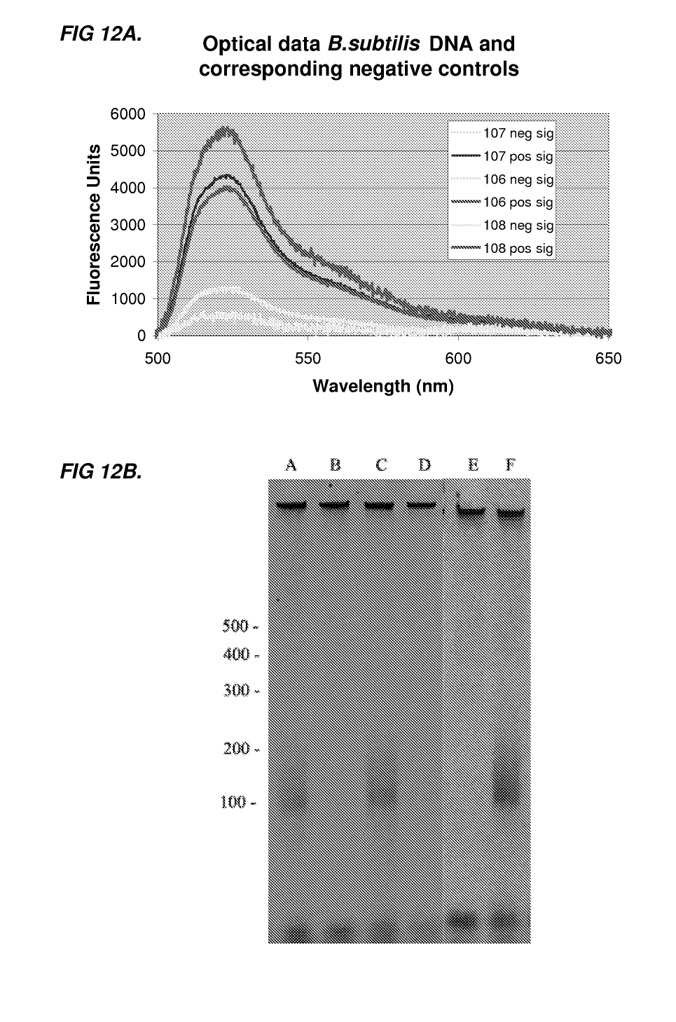

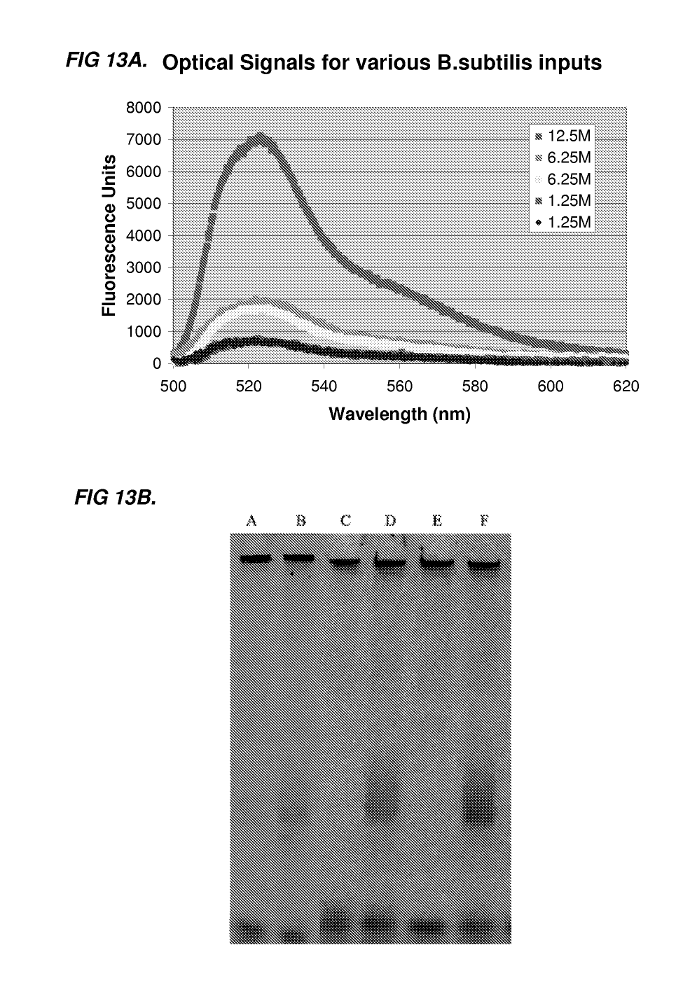

Shown in FIG. 1 are different embodiments of controlling the rate and/or velocity of fluid flow through the valves connected to an input and/or output port at the interface with the microfluidic device. In some embodiments, a valve at the interface is connected to a pump which is connected to a tank, where the pump pumps fluid from the tank at pre-determined flow rate to the valve at the interface. The valve connected to the pump can be used for low flow rate (resulting in low velocities on chip) or high flow rate (resulting in high velocities on chip) flow input into the microfluidic device. In some embodiments, a valve at the interface is connected to a flow restrictor. A tank is connected to both a valve and the flow restrictor, and in some embodiments, the flow restrictor can establish a pre-determined pressure in the tank, and where fluid flows from flow restrictor to the valve which is fluidly connected at the interface. The valve connected to flow restrictor can be used for low flow rate or preferably high flow rate flow input into the microfluidic device. In another embodiments, a valve at the interface is connected to a tank which is connected to another valve, where fluid flows from tank to the valve which is fluidly connected at the interface. In some embodiments, a valve which interfaces with the microfluidic device is connected to air, for example, for input and/or output of air into the microfluidic device. In some embodiments, a valve at the interface is connected to an output port on the microfluidic device, where the valve is fluidly connected to a waste tank or chamber. In some embodiments, the system comprises at least one sample analysis/detection subsystem, which is connected to the computer. In some embodiments, the sample analysis detection subsystem connects to analysis interface which contacts the microfluidic chip for analysis of samples “on-chip”. In alternative embodiments, the sample analysis/detection subsystem can be used for the analysis of a sample “off-chip”, where the sample is collected in an end-sample container off chip. In some embodiments, the control unit connects to a thermal controller, which is connected and controls the temperature of the thermal interface which interfaces with an integrated microfluidic chip. The thermal interface typically only interfaces with a region or part of the microfluidic chip which comprises PCR channels (see FIG. 5).

FIGS. 2A-2B shows embodiments of the remote valve switching system comprising the control unit, optical subsystem and thermal control subsystem and its interface with the microfluidic device. FIG. 2A is a schematic drawing of an embodiment of the instrument functionality and photo of the integrated microfluidic chip (comprising speed-changing flow reservoir buffers) and the instrument interface. The interface block, which fluidly connects the permanent off-chip remote valves to input and/or output ports of the microfluidic chip is raised to show the position of the microfluidic chip (arrow).

In some embodiments, the interface block comprises the permanent off-chip remote valves. The interface block is configured to fluidly connect to as many input/output ports on the microfluidic device, for example, at least one, or at least 2, or at least 3, or at least 4, or at least 5, or at least 5-8, or at least 5-10, or at least 10-15 or at least 15-20, or at least 20-25, or at least 25-30, or at least 30-35 or at least 35-50 or more than 50 permanent off-chip remote valves switching devices to input and/or output ports of the microfluidic chip, can be lowered to contact the microfluidic chip, such that the remote valve switching devices are in fluid contact with input/output ports of the microfluidic chip. The location of the permanent off-chip remote valves switching devices connected to the interface block, and the interface block is configured in such a way that when the interface block is lowered and contact the microfluidic chip, each of the remote valve switching device fluidly connects 300 with the external surface or chip boundary 340 of the microfluidic device 260 so that the channel 310 of remote valve switching device is in fluid communication with the channels of the microfluidic chip 260 (See FIG. 10). FIG. 2 B shows a photograph of instrument. The black square box highlights the housing door of the microfluidic chip/instrument interface housing.

FIGS. 3A-3C show schematics of an embodiment of the configuration of an integrated microfluidic device comprising a reservoir for change of velocity from a high- to a low-velocity. FIG. 3A shows high fluid flow through a microfluidic chip with a reservoir in the high- to low flow configuration. In particular the valve configuration of the remote valve switching system for fluid flow for at high velocities for high flow processes, where the microfluidic chip comprises a reservoir after the high flow chamber (e.g. mixer) and before the low flow chamber (e.g. SPE column). In FIG. 3A, the valves are configured for fluid flow at high velocities for high flow processes (e.g. mixing), where input valves (V_{I}), V_{I1} and V_{I2} are in the open position so that at least one fluid sample flows at high rate from output ports of the remote valve switching system into the input ports of the microfluidic chip and into the mixer and into the reservoir on the microfluidic device. Due to the high resistance of the low flow chamber (e.g. SPE column) and because the exhaust valve (V_{E}), V_{E1} is in the open configuration, fluid is directed (see arrow) from the reservoir to an output port at the interface with the remote valve switching system. The exhaust valve (V_{E}), V_{E2} is in the closed configuration to create a “liquid stopper” to direct the flow of fluid away from the low flow chamber (see direction of arrow). In alternative embodiments, where it is not necessary to mix more than one fluid, only one input valve (V_{I1}) is necessary and when the input valve V_{I1} is in the open position the fluid flows at high rate from output ports of the remote valve into the input ports of the microfluidic chip and into the mixer and into the reservoir on the microfluidic device.

FIG. 3B shows low fluid flow through a microfluidic chip with a reservoir in the high- to low-flow configuration. In particular, the valves are configured for fluid flow for at low velocities for low flow rate processes (e.g. nucleic acid extraction using the SPE column), where input valve V_{I1} is in the open position, and V_{I2} and exhaust valve V_{E1} in the closed position, so that at least one fluid sample, typically a propulsion buffer, flows at low rate from the input valve V_{I1} to the input port of the microfluidic chip, and is directed (as shown by an arrow) through the mixer and displaces fluid from the reservoir into the low flow chamber (e.g. SPE column). As V_{E1} in the closed position, it creates a liquid stopper directing (as shown by the arrow) the fluid displaced from the reservoir into the low flow chamber (e.g. SPE column). Fluid is directed along the microfluidic channels and through any downstream components, e.g. in some embodiments, PCR channels, and as V_{E2} in the open position, the sample flows into a waste collection either on or off-chip. In some embodiments, the sample is collected on-chip, and analysis performed on-chip at the optical/analysis interface (see FIG. 1).

FIG. 3C shows a flow diagram of the method steps for an embodiment using a microfluidic reservoir configuration of FIGS. 3A and 3B change the flow from a high flow to a low flow rate, using the remote valves to control directional fluid flow on the microfluidic device.

FIGS. 4A-4B show schematics of an embodiment of the configuration of an integrated microfluidic device comprising a reservoir for change of velocity from a low- to a high-velocity. FIG. 4A shows low fluid flow through a microfluidic chip with a reservoir in the low- to high flow configuration. In particular, FIG. 4A shows the valve configuration of the remote valve switching system for fluid flow for at low velocities for low flow processes, where the microfluidic chip is configured such that a reservoir is positioned after (downstream) of a low flow chamber (e.g. SPE column) and before a high flow chamber (e.g. mixer). In FIG. 4A, the valves are configured for fluid flow at low velocities for low flow rate processes (e.g. nucleic acid extraction using SPE), where input valves (V_{I}), V_{I1} is in the open position and V_{I2} is in the closed configuration, so that fluid flows at low flow rate from output ports of the remote valve switching system into the input ports of the microfluidic chip and into the low flow chamber (e.g. SPE column) and into the reservoir on the microfluidic device. As V_{I2} in the closed position, it creates a liquid stopper directing the fluid from the low flow chamber into reservoir (as shown by the arrow). Fluid displaced from the reservoir can exit the microfluidic device via an open Exhaust valve, V_{E1} (V_{E2} is closed). Once all the sample fluid has been processed by the low flow chamber and is present in the reservoir, the configuration of the valves can be changed by the remote valve switching system so that the sample can proceed to be processed by high flow processes (e.g. mixers and the like). For example, a change of valve configuration so that input valves V_{I1} is closed and V_{I2} is opened, and exhaust valve V_{E2} remains closed (the valve configuration for high flow in low-to-high flow microfluidic chip configuration is not shown). With the valves in this high-flow configuration, a propulsion buffer can be flowed at high flow rate from V_{I2}, displacing the fluid in the reservoir which flows into a downstream high flow chamber (e.g. mixer). As V_{I1} is in a closed configuration, and/or the high resistance of the low flow chamber, fluid inflowing from V_{I2} will be directed into the reservoir chamber (not shown). In some embodiments, it may be desirable to mix the sample displaced from the reservoir with a second fluid sample, e.g. a PCR master mix, in which case, V_{I2} and V_{E1} can both be in an open configuration, and simultaneously allow inflow of propulsion buffer at high flow rate from V_{I2} with inflow of the second fluid (e.g. a PCR master mix) through V_{E1} at the a high flow rate, allowing the displaced sample from the reservoir to mix with the second fluid (e.g. PCR master mix) and enter the mixer at high velocity (not shown). Shown is a channel whereby the sample flows from the reservoir to the exhaust valve V_{E1}. However, in some embodiments like in FIG. 5A, a PCR chamber (e.g. PCR channels) or other components are downstream of a high flow chamber (e.g. mixer) in a “low-flow-reservoir-high-flow” microfluidic chip configuration, in which case sample from the mixer can be processed by PCR, using the thermal interface controlled by the thermal controller, as shown in FIG. 1. The end sample can be analyzed when still present on the microfluidic chip, for example by a sample analysis/detection system connected to the computer, as shown by FIG. 1. In an alternative embodiment, the end sample can be analyzed off the chip by a sample analysis/detection system connected to the computer, then the sample it is transferred to an end sample collection chamber (see FIG. 1.). In alternative embodiment, the end sample can be analyzed on-chip, however, the microfluidic chip can be removed from system and the remote-valve interface, allowing the microfluidic chip to be analyzed by an independent sample analysis/detection system. In another embodiment, the sample is removed from the microfluidic device and transferred to a separate container for analysis, for example, a multi-well plate (e.g. 96, 384-well plates) for high-throughput analysis of multiple samples simultaneously. This is advantageous for comparing multiple samples at the same time for comparison and accuracy purposes, as well as reduces the time each microfluidic chip is in the system of the invention, thus increasing efficiency and throughput of the system.

FIG. 4B shows a flow diagram of the method steps for an embodiment using a microfluidic reservoir configuration of FIG. 4A to change the flow from a low to a high flow, using the remote valves to control directional fluid flow on the microfluidic device.

FIGS. 5A-5C shows one embodiment of an integrated disposable microfluidic chip comprising speed-changing fluid reservoirs. FIG. 5A shows one embodiment of the chip design with fluid inputs and outputs and functional regions labeled. The sample is placed into an on-chip sample reservoir via a sample in input port 10. The sample reservoir is fluidly connected to a reservoir in a high- to low flow configuration (see FIGS. 3A and 3B). For example, the sample reservoir 15 is connected to a first high flow chamber (e.g. a mixer) 30 which is connected to a first reservoir 40 which is fluidly connected to low flow chamber (e.g. SPE column) 50. The low flow chamber (e.g. SPE column) 50 also forms part of low- to high-flow reservoir configuration (see FIG. 4A), where the low flow chamber (e.g. SPE column) 50 is in fluid communication a second reservoir 100 which is connected to a second mixer 110. The second mixer is connected to a PCR channel 130 which is connected to an on-chip end sample detection well 140. FIG. 5B shows a photograph of one such embodiment of a chip prototype demonstrating its planar configuration, disposable format in a credit card-like size.

FIG. 6 is a schematic of one embodiment of a high flow chamber, such as a zig-zag mixer 30, showing inputs 160 and 170 and an output 190 which fluidly communicates with a reservoir. In one embodiment, this represents the configuration of the mixer in a high- to a low-flow reservoir configuration, where input stream receives propulsion buffer from a valve of the remote valve switching system and the other input stream receives the fluid test sample, e.g. from the on-chip the sample reservoir 15.

FIG. 7 is a schematic of one embodiment of a reservoir-buffer chamber in a configuration to decrease the flow from a high to a low flow. FIG. 7 shows the input 160 and output 190 of the high flow chamber (i.e. a mixer 30), where the output of the mixer is fluidly connected to the input 200 of the reservoir 40. The output of the reservoir 230 is fluidly connected to a low flow chamber, such as a PPM (porous polymer monolith) 50, and an exhaust valve 260.

FIG. 8 is a schematic of one embodiment of the reservoir-buffer chamber as shown in FIG. 7, with the exhaust valve in an open configuration, allowing a mixed sample to enter the reservoir 40 at a pre-determined or designated high flow velocity for sufficient mixing and displace a holding solution in the reservoir into waste chamber off-chip (not shown). The predetermined low flow rate is controlled by the fluid input into the remote valves, which is regulated by the control unit.

FIG. 9 is a schematic of the reservoir-buffer chamber as shown in FIG. 7, which is the next step from that shown in FIG. 8, with the exhaust valve in a closed configuration, allowing a propulsion buffer at a specified low flow rate to displace the sample in the reservoir into the low flow chamber, such as the SPE or PPM column 50. The predetermined low flow rate is controlled by the fluid input into the remote valves, which is regulated by the control unit.

FIG. 10A-10C show schematic drawings of an embodiment of the control of directional fluid flow of a channel in a microfluidic device by two remote-valve devices. FIG. 10A shows a schematic of the top view of the principal of directional fluid flow onto a microchip via remote valve switching, where one inlet valve V_{I1} is in the open configuration, and V_{I2} is in the closed configuration, so that the channel connecting to V_{I2} acts as a liquid stopper, thus directing the direction of the fluid flow on the microfluidic chip. FIG. 10B shows a schematic of the top view of the principal of directional fluid flow of a microchip via remote valve switching, where one exhaust valve V_{E1} is in the open configuration, and V_{E2} is in the closed configuration, so that the channel connecting to V_{E2} acts as a liquid stopper, thus directing the direction of the fluid flow on the microfluidic chip. In an alternative embodiment, V_{E2} were open, and V_{E1} closed, then the channel arm of the microfluidic chip connected to V_{E1} would act as a liquid stopper, and fluid would be directed to the exhaust valve V_{E2}. FIG. 10C shows a schematic of the side view of remote valve switching system and a microfluidic chip. Shown are the interface fluidly connecting the remote valves to inlet and outlet ports on the top surface of a microfluidic device, however, in alternative embodiments, the valves can be fluidly connected to an interface which connects to inlet and outlet ports on the side of the microfluidic device.

FIGS. 11A-11B show representative on-chip measuring of the on-chip temperature of a dummy chip using the thermocouple thermal interface. FIG. 11A shows an example on-chip thermal profile measured with a thermocouple embedded in a dummy chip. FIG. 11B shows an optical detector calibration dilution curve using a 90 bp (basepair) amplicon generated off-chip using a B. subtilis Taqman assay. The amplicon concentration was estimated at 8.1 μg/mL using an absorbance measurement, and the fluorescence measurements of serial dilutions of the target were conducted on-chip and the peak fluorescence is graphed for the various amplicon concentrations. The on-chip detection can be measured at less than 1 ng/μL amplicon. Mean values of 50 measurements and error bars (one standard deviation) are plotted.

FIGS. 12A-12B show the optical detection data for fully integrated chip protocol with an input of 20 ng of B. subtilis DNA. Corresponding negative controls (sample of water) are shown. Legend indicates arbitrary chip number followed by a designation of “neg sig” for the water input and “pos sig” for the 20 ng DNA sample. FIG. 12B shows results of an example gel electrophoresis of amplicon (90 bp). A: Chip #107 positive, B: Chip #107 negative, C: Chip #106 positive, D: Chip #106 negative, E: Chip #108 negative, F: Chip #108 positive.

FIGS. 13A-13B show functionality of the remote valve system and integrated microfluidic chip comprising reservoirs to detect presence of bacteria in a sample. FIG. 13A shows optical detection for various numbers of B. subtilis cells; Red: 12.5×10⁶ cells, Yellow and Orange: 6.25×10⁶ cells, Green and blue: 1.25×10⁶ cells. The values shown are normalized against respective negative control samples. FIG. 13B shows a representative gel electrophoresis of resultant on-chip generated amplicons (90 bp). A: Negative control for 1.25×10⁶ cells experiment, B: 1.25×10⁶ cells input, C: Negative control for 6.25×10⁶ cells experiment, D: 6.25×10⁶ cells input, E: Negative control for 12.5×10⁶ cells experiment, F: 12.5×10⁶ cells input.

### DETAILED DESCRIPTION OF THE INVENTION

The proposed invention generally relates to the improvement of fluid flow on microfluidic chips and devices. Herein, the inventors demonstrate use of a simple, disposable integrated microfluidic device which comprises reservoirs to serve as fluid buffers to control the flow rate of fluids through the device, thus enabling both high flow processes and low process to be present on the same microfluidic chip, and where the direction of the fluid is controlled by off-chip, remote switching valves. As the microfluidic chip does not comprise any on-chip valves, it can be produced at very low manufacturing costs, yet maintaining both high-flow and low flow components on the same microfluidic device, thus reducing sample transfer issues (e.g. contamination and sample loss) and enabling a fully integrated microfluidic chip for high flow and low flow processes on a single chip which is disposable.

Additionally, the remove valve switching mechanism which interfaces with the microfluidic device can be used with any generic microfluidic chip which is configured to use the same interface. As such, the present invention provides a dynamic and diverse system in which fluid flow on the microfluidic chip can be controlled remotely, and in embodiments where the microfluidic chip comprises reservoirs to buffer fluid flow from a high to a low flow process, and vice versa, the microfluidic chip can process the sample through multiple manipulations on one chip, such that the chip can be tailored and selected for a specific function or assay (e.g. nucleic acid assay, PCR assay, immunoassay etc.).

Stated another way, the present invention relates to a system where a change in direction of fluid flow on a microfluidic chip is controlled off-chip using a remote valve switching system, and a change in rate and/or velocity of fluid flow on the microfluidic chip is controlled on-chip using reservoir buffers. This is a highly diversifiable system, enabling fluid flow through a disposable integrated microfluidic chip, (e.g. a microfluidic chip comprising components requiring both high- and low flow rates for different processes to be performed on a single chip) to be controlled in an automated manner, without the need of expensive on-chip valves. As the disposable integrated microfluidic chip is valve-less, it can be produced at easily and rapidly with low manufacturing costs.

One aspect of the present invention therefore relates to a remote valve switching system, connected to a control unit for directional fluid control of fluid on a microfluidic device which lacks valves or pumps on the microfluidic chip. Accordingly, one aspect of the invention relates to a remote-valve microfluidic system.

Another aspect of the present invention relates to control of rate and/or velocity of fluid flow, and in particular to allowing flow rate and/or velocity changes on a valveless microfluidic device. According to this aspect of the invention, the inventors incorporated flow-rate and/or velocity changing fluid-reservoirs, herein referred to as “reservoir-mixer buffers” or “reservoirs” to increase or decrease the fluid flow rate and/or velocity on a microfluidic device. Accordingly, one aspect of the invention relates to a microfluidic device comprising at least one reservoir to change (i.e. increase or decrease) the flow rate and/or velocity of the fluid on the microfluidic chip. In some embodiments, the reservoirs can be used to change the flow from a high- to a low flow, or alternatively, to change the flow from a low to a high-flow. As such, the reservoir comprising microfluidic chips enable samples to be processed through multiple components on a single integrated chip which required different flow rates, without the need of expensive on-chip valves and the like.

Accordingly, the present invention provides an integrated valveless microfluidic device where directional fluid control is controlled using remote valve switching and fluid flow rate/velocity changes are controlled using flow changing fluid reservoirs. In particular, different embodiments of the present invention provides two methods to control fluid flow in a microfluidic device; (i) a remote-valve switching device to control directional fluid flow and (ii) a reservoir-buffered mixer to control fluid flow rate or to change the fluid speed on a microfluidic device. In some embodiments, a remote-valve switching device and a reservoir-buffered mixer can be used together or independently on a microfluidic device to improve fluid flow in microfluidic devices.

Accordingly, one aspect of the present invention relates to a Remote-valve switching device. In one embodiment of this aspect of the present invention, a remote-valve switching device is an off-chip valve that functions as a remote-valve switching device to direct the flow of a fluid on a microfluidic chip. Accordingly, a remote valve device eliminates the need for valves to be present on a disposable chip (e.g. eliminates on-chip valves) which are often expensive and complicated to produce for microfluidic devices desired to be disposable, and which need to be manufacturer at low cost.

In one embodiment, when the off-chip valve is in the closed position, fluid cannot flow through the channel which the off-chip valve is controlling, and thus the fluid is diverted along a different channel at a junction located closest to the off-chip valve. In some instances, the fluid is diverted to the closest outlet port with an open valve. When a remote valve is in an off position, it blocks fluid flow of a port of an input/output channel of the microfluidic chip, therefore diverting fluid along the direction of another channel at an upstream junction point (see FIGS. 3 and 4 for examples). Accordingly, an off-chip valve works because the flow path between the junction and the off-chip valve functions as a “liquid stopper”, blocking any flow along that leg of the junction controlled by the off-chip valve. Accordingly, one aspect of the present invention relates to an off-chip permanent valve which functions as remote-valve actuation system.

In one embodiment of the remote valve switching device, an off-chip valve comprises the following elements: a valve with an input and an output, where the input of the valve is connected to an arm channel of an on-chip junction, and where the valve can be in an open or closed configuration. Stated another way, a valve can be a simple valve, or any valve known by one of ordinary skill in the art whereby if it is in the open configuration, fluid is allowed to flow through the valve, and when the valve is in the closed configuration, fluid is prevented to flow through the valve.

If the valve is in the closed configuration, fluid is prevented from flowing through the valve. As a result, fluid fills the channel on the chip connected to the valve up to the point of a junction in the channel (e.g. acts as liquid stopper), and flow through microfluidic device is diverted along an alternative channel route. If a remote-valve is in the open configuration, fluid can flow through the valve in either direction, depending on where the fluid pressure is higher, and where the fluid flows from the high pressure to low pressure direction. In some instances, the fluid flows from a pump through a remote valve and to an input port of the microfluidic device at the interface (e.g. see input valve V_{I1} in FIG. 3A), or vice versa, e.g. fluid flows from a channel in the microfluidic device and out of an output port at the interface and to a remote valve) (e.g. see exhaust valve V_{E1} in FIG. 3A). In some embodiments, fluid impermeable material, for example, rubber o-rings, or other equivalent sealing mechanism are placed at the interface at the junction on the microfluidic chip, where the valve is connected to the input or output port of a channel of a microfluidic device. In alternative embodiments, the material at the interface junction between the microfluidic chip and the interface block can be any material commonly known to persons of ordinary skill in the art which prevents fluid leakage, for example, elastomer film and the like.

Another aspect of the present invention relates to a reservoir-buffered mixer. In one embodiment of this aspect of the present invention, a reservoir-buffered mixer is a fluid-collection and holding reservoir which is located between two chambers which require different flow rates (e.g., a reservoir can be located between a slow flow chamber (e.g. a slow column such as a DNA extraction column) and a chamber requiring a high fluid velocity, e.g. such as a fluid mixer column). Accordingly, the reservoir-buffered mixer allows processes requiring both high flow and low flow rates to be carried out on the same single integrated microfluidic chip.

In some embodiments of this aspect of the invention, the reservoir is located between columns or channels in the microfluidic device which require different flow rates (i.e. a high flow mixing column and a low-flow processing column). As an illustrative example only, where it is desirable to change the flow from a high to a low flow (i.e. decrease flow rate and/or velocity), the following configuration can be used, for example, high flow chamber which is fluidly connected to a reservoir chamber, which is fluidly connected to a low flow chamber (e.g. see FIGS. 3A and 3B).

In an alternative embodiment, where it is desirable to change the flow from a low flow to a high flow (i.e. increase flow rate and/or velocity) the following confirmation can be used, for example, a low flow chamber which is fluidly connected to a reservoir chamber, which is fluidly connected to a high flow chamber (e.g. see FIG. 4A).

For example, in one embodiment, a reservoir can be located between the mixing chamber (which requires high flow velocity) and a separation column (such as a PPM or SPE column) which requires a low flow velocity. Accordingly, the inventors have discovered a method to change the flow rate and/or velocity in a microfluidic device from a high-flow to a low-flow. In such an embodiment, the reservoir collects the fluid from the output of a high-flow chamber (e.g. a mixing chamber) and allows input at a defined slow flow into a slow flow chamber, e.g. a separation column (i.e. PPM column) (see FIGS. 3A and 3B).

In some embodiments, a reservoir-buffered mixer present on a microfluidic device can be used to change the fluid flow from a high to a low flow rate and/or velocity. In such an embodiment, a microfluidic chip is configured so the reservoir is positioned to decrease the fluid flow from a high flow, where the methods steps are as follows:
1. the reservoir comprises a holding solution, and the output of the reservoir is controlled by an exhaust valve, controlling output of the fluid from the reservoir into a waste chamber.
2. When the exhaust value is in the open configuration, it allows the holding solution in the mixer and reservoir to be expelled into a waste compartment. When two fluids from two input streams are converged into a single output stream and passed through the mixer channel (such as a zigzag-mixer channel) at high flow velocity, and the output stream of the mixer column passes into the reservoir at a high flow velocity and rate and displaces the holding solution into an waste reservoir.
3. When the exhaust valve is in the closed configuration, a propulsion buffer of an input stream displaces the fluid in the reservoir at a low flow rate/velocity into the separation column (i.e. PPM column).

In another embodiment, a reservoir-buffered mixer present on a microfluidic chip can be used to increase fluid rate and/or velocity from a low to a high flow. In such an embodiment, a reservoir can be located between a low flow column, such as for example a separation column (i.e. PPM column) which requires a low flow rate and low velocity and a high flow channel, (e.g. such as a mixing chamber) which requires high velocity for optimal mixing. Such an embodiment is useful where it is desirable to mix the output fluid from the low flow column (i.e. the separation column) with additional agents (such as preservatives, diluants, and/or PCR reagents etc).

Accordingly, one aspect of the reservoir-buffered mixer aspect of the invention relates to use of the reservoir to change the flow rate and/or velocity in a microfluidic device from a high to a low flow condition, for example where the reservoir is located after a high flow velocity column such as for example a mixer column, but before the low flow velocity column, such as a separation column. In such an embodiment, the configuration of the reservoir-buffered mixer on the microfluidic device comprises the following elements: (i) a reservoir, with an input and an output. (ii) a mixer chamber (such as a zig-zag shaped chamber or any other known mixer) with an input and an output, wherein the output of the mixer is connected to the input of the reservoir, (iii) a waste chamber, the input of the waste chamber connected to the output of the reservoir, (iv) an exhaust valve which controls flow of the fluid from the output of the reservoir to the input of the waste chamber.

In another embodiment of the reservoir-buffered mixer aspect of the invention relates to use of the reservoir to change the flow rate and/or velocity in a microfluidic device from a low to a high flow, for example where the reservoir is located after a low flow column, such as a separation column but before high flow velocity column such as a mixer column. In such an embodiment, the configuration of the reservoir-buffered mixer on the microfluidic device comprises the following elements: (i) a reservoir, with at least one input and an output, where one input is connected to a low flow column (i.e. a separation column) and a second input is for high flow propulsion buffer; (ii) a mixer chamber (such as a zig-zag shaped chamber or any other known mixer) with an input and an output, wherein the input of the mixer is connected to the output of the reservoir.

Other aspects of the invention relate to the use of the reservoir situated between any two or more elements on a microfluidic chip which require different flow rates. In some embodiments, a microfluidic device can comprise any number of reservoir situated between and connecting a high flow channel and a low flow channel on a microfluidic device, for example a microfluidic device can have at least one or at least 2 or at least 3 or at least 4 or at least 5, or at least 6, or at least 7, or at least 8, or at least 9 or at least 10 or more than 10 reservoirs situated between and connecting a high flow channel and a low flow channel on a microfluidic device.

In some embodiments of all aspects of the present invention, a high flow element is a mixer column designed to mix at least one fluid with a second or more fluid. In other embodiments, a high flow column is any high flow element on a microfluidic device, including mixers or lysis columns and the like. In some embodiments, a mixer element can be in any confirmation of a mixer element which requires a high flow, such as zigzag configuration, coanda effect channels, v-shaped bas-relief shaped mixers, other micromixer configurations such as slanted groove, staggered herringbone and herringbone mixers and other mixers known in the art.

In some embodiments of all aspects of the present, a low flow element is any low flow column known to a skilled artisan, for example a separation column, including but not limited to separation columns, PPM columns etc.

In some embodiments of all aspects of the present invention, the reservoir-buffered mixer can comprise a reservoir of any shape or size, or geometric configuration, for example, but not limited to a serpentine, zig-zag, coils configuration or any configuration to minimize the space it takes up on the microfluidic chip. In some embodiments, the size and volume of the reservoir is dependent on the volume required for the preceding or subsequent step (i.e. for the high-flow step or the low-flow step)

### DEFINITIONS

The term “flow rate” refers to the volume of fluid that passes through any cross section of the channel per unit of time. “Flow rate” is a measure of fluid volume per volume/unit time, and is typically measured in μL/second or μL/min. Flow rate and flow velocity are related through the cross-sectional area of the channel. Whereas some processes require a particular velocity, often the flow rate is controlled by pumps and velocity by the geometry of the channel.

The term “velocity” refers to a measure of fluid flow over a defined distance per unit of time, and is typically measured in mm/sec or mm/min, and refers to the speed of the molecules of fluid. Velocity is important for many of the processes, the mixing channels (e.g. high flow channels) and the SPE columns (e.g. low flow chambers). Velocity of a fluid in a channel is dependent on channel cross sections.

The term “low flow rate” refers to a flow rate of less than 200 μL/minute, and can be any range from 0.0001 μL/min to 199.99 μL/min. In some embodiments herein, a low flow rate is for example, but not limited to, the rate required for a particular biological process to be effective, such as DNA extraction using a SPE column as disclosed herein. A slow flow rate is at a lower flow rate than a high flow rate, for example at least 10% as compared to a high flow rate, for example a decreased flow rate by at least about 20%, or at least about 30%, or at least about 40%, or at least about 50%, or at least about 60%, or at least about 70%, or at least about 80%, or at least about 90% or up to and including a 100% decrease, or any decrease between 10-100% as compared to a high flow rate. In some embodiments, a low flow rate is approximately a volume per minute of 2 or 3 or 4 or 5 to 10 times slower that the flow rate of a high flow element as that term is defined herein. In some embodiments, a flow rate of a low flow element is a flow rate of less than 1 mL/min or flow rate below, for example, in the range of 0-199.99 μL/min, or for example within the range of 0.1 μL/min to 50 μL/min, or in the range of 50 μL-100 μL/min or in the range of 100-199.99 μL/min or any range therebetween.

The term “low velocity” refers to a velocity of less than 155 mm/sec, and can be any range from 0.0001 mm/sec to 149.99 mm/second. In some embodiments, a low velocity rate refers to a velocity of between 15-150 mm/second. In some embodiments herein, a low velocity is for example, but not limited to, the rate required for a particular biological process to be effective, such as DNA extraction using a SPE column as disclosed herein. A slow velocity is at a lower velocity than a high velocity, for example at least 10% as compared to a high velocity, for example a decreased velocity by at least about 20%, or at least about 30%, or at least about 40%, or at least about 50%, or at least about 60%, or at least about 70%, or at least about 80%, or at least about 90% or up to and including a 100% decrease, or any decrease between 10-100% as compared to a high velocity. In some embodiments, a low velocity is approximately a mm/second 2 or 3 or 4 or 5 to 10 times or 100 times slower that the velocity of a high flow element as that term is defined herein.

The term “high flow rate” refers to a rate of 200 μL/min or a flow rate greater than 200 μL/min. In some embodiments, a high flow rate is a flow rate of a flow volume per minute of 2 or 3 or 4 or 5 to 10 times faster that the flow rate of a low flow element as that term is defined herein. In some embodiments, a flow rate of a high flow element is a flow rate of 200 μL/min or any rate above 200 μL/min, for example, at least about 400 μL/min, or at least about 500 μL/min, or at least about 1 mL/min or at least about 1.2 mL/min, at least 1.5 mL/min, at least 2 mL/min above. In some embodiments, a high flow rate has a faster flow rate of at least about 20%, or at least about 30%, or at least about 40%, or at least about 50%, or at least about 60%, or at least about 70%, or at least about 80%, or at least about 90% or up to and including a 100% faster or any faster rate between 10-100% as compared to a reference flow rate (i.e. a reference slow flow rate), or at least about a 2-fold, or at least about a 3-fold, or at least about a 4-fold, or at least about a 5-fold or at least about a 10-fold faster rate, or any increase between 2-fold and 10-fold or greater than 10-fold faster flow rate as compared to a reference flow rate (i.e. a reference slow flow rate).

The term “high velocity” refers to a velocity of greater than about 155 mm/sec, and can be any range from 150 mm/sec to 1500 mm/second or greater than 1500 mm/second. In some embodiments, a high velocity rate refers to a velocity of between 150-1500 mm/second. In some embodiments, a velocity of a high flow element has a velocity of greater than 150 mm/sec or any rate above 150 mm/sec, for example, at least about 200 mm/sec, or at least about 300 mm/sec, or at least about 400 mm/sec, or at least about 500 mm/sec, at least 600 mm/sec, or at least 800 mm/sec mL/min, or at least about 1000 mm/sec, or at least about 1200 mm/sec, or at least about 1500 mm/sec or above. In some embodiments, a high velocity has a faster velocity of at least about 20%, or at least about 30%, or at least about 40%, or at least about 50%, or at least about 60%, or at least about 70%, or at least about 80%, or at least about 90% or up to and including a 100% faster or any faster velocity between 10-100% as compared to a reference flow velocity (i.e. a reference slow velocity), or at least about a 2-fold, or at least about a 3-fold, or at least about a 4-fold, or at least about a 5-fold or at least about a 10-fold faster rate, or any increase between 2-fold and 10-fold or greater than 10-fold faster velocity as compared to a reference velocity rate (i.e. a reference slow velocity).

As used herein, a “device” refers to a tool or piece of equipment which typically is used for a particular function, mechanical task or use, for example, in some embodiments of the present invention, the device is used as a tool for controlling fluid flow (i.e. directional or rate of fluid flow) as described herein.

The term “microfluidics” or “microfluidic device” or “microfluidic chip” are used interchangeably herein and refer to the manipulation of microliter and nanoliter volumes of fluids and the design of systems in which such small volumes of fluids will be used.

The term “Lab-on-a-chip” as used herein refers to a platform to perform laboratory reactions and processes on a single microfluidic chip on a micro-scale level. Typically, lab-on-a-chip are relatively inexpensive disposable chips that do not require highly skilled personnel or expensive laboratory space, and which allow processing of a small amount of sample material. In some embodiments, the lab-on-a-chips enable processing of a sample sequentially through multiple reactions and/or processes using a single device. Lab-on-a-chip devices are typically designed to perform a particular laboratory reaction, for example extraction and isolation of biomolecules from a biological sample. A lab-on-a-chip used herein encompasses all microfluidic devices which enable processing of samples in small volume and on a micro-scale, and includes, without limitation, lab-on-chips which takes an unprocessed sample and processes the sample through multiple processes, including end-product sample production and sample detection.

The terms “lower”, “reduced”, “reduction” or “decrease” or “slower” are all used herein generally to mean a decrease by a statistically significant amount. However, for avoidance of doubt, “lower”, “reduced”, “reduction” or “decrease” or “inhibit” means a decrease by at least 10% as compared to a reference level (i.e. a reference flow rate), for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level (i.e. a reference flow rate).

The terms “increased”, “increase” or “enhance” or “higher” or “faster” are all used herein to generally mean an increase by a statically significant amount; for the avoidance of any doubt, the terms “increased”, “increase” or “enhance” or “higher” or “faster” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level (i.e. a reference flow rate), or at least about a 2-fold, or at least about a 3-fold, or at least about a 4-fold, or at least about a 5-fold or at least about a 10-fold increase, or any increase between 2-fold and 10-fold or greater as compared to a reference level (i.e. a reference flow rate).

The term “high flow chamber” refers to any element, component or channel on a microfluidic device which has a preferred flow rate above about 200 μL/min or any rate above about 500 μL/min or any rate above about 1 ml/min or any rate above 1 mL/min, for example, at least 1.2 mL/min, at least 1.5 mL/min, at least 2 mL/min. In some embodiments, a high flow rate is a volume per minute above 2 or 3 or 4 or 5 to 10 times faster that the flow rate of a low flow chamber to which is fluidly connected to an intermediate reservoir chamber.

The term “low flow chamber” refers to any element, component or capillary channel on a microfluidic device which has a preferred flow rate of less than 200 μl/min flow rate or any rate below, for example, in the range of 0-199.9 μL/min, or for example within the range of 0.1 μL/min to 10 μL/min or 1.0 μL/min to 50 μL/min, or in the range of 50 μl-100 μL/min or in the range of 100-199.9 μL/min or any range therebetween. In some embodiments, a flow volume per minute of 2 or 3 or 4 or 5 to 10 times slower that the flow rate of a high flow chamber to which is fluidly connected to an intermediate reservoir chamber.

The term “exhaust valve” refers to a valve which regulates the flow of a capillary channel connected to an outlet of reservoir, wherein when the exhaust valve is open it allows fluid to flow from the reservoir, as it is being displaced by a different fluid (i.e. a sample fluid) to an exhaust chamber or a waste chamber.

The term “inlet” is the passageway of fluid into a chamber or channel.

The term “outlet” is the passageway of fluid out of a chamber or channel.

The term “eluant” or “eluted sample” as used herein refers to a sample that is collected after processing with at least one module of the microfluidic device.

The term “microchannel” as used herein, refers to a channel that is sized for passing through microvolumes of liquid.

The term “channel” as used herein means any capillary, channel, tube or grove that is deposed within or upon a substrate.

The term “microorganism” as used herein includes ay microscopic organism or taxonomically related organisms within the categories of bacteria, algae, fungi, yeast, protozoa and the like. The microorganisms targeted can be pathogenic microorganisms.

The term “bacteria” as used herein is intended to encompass all variants of bacteria, for example, prokaryotic organisms and cyanobacteria. Bacteria are small (typical linear dimensions of around 1 μm), non-compartmentalized, with circular DNA and ribosomes of 70S. The term bacteria also includes bacteria subdivisions of Eubacteria and Archaebacteria. Eubacteria can be further subdivided on the basis of their staining using Gram stain, and both gram-positive and gram-negative eubacteria, which depends upon a difference in cell wall structure are also included, as well as classified based on gross morphology alone (into cocci, bacilli, etc.).

The term “pathogen” as used herein refers to any disease producing microorganism.

The term “pathology” as used herein, refers to symptoms, for example, structural and functional changes in a cell, tissue, or organs, which contribute to a disease or disorder. For example, the pathology may be associated with a particular nucleic acid sequence, or “pathological nucleic acid” which refers to a nucleic acid sequence that contributes, wholly or in part to the pathology, as an example, the pathological nucleic acid may be a nucleic acid sequence encoding a gene with a particular pathology causing or pathology-associated mutation or polymorphism. The pathology may be associated with the expression of a pathological protein or pathological polypeptide that contributes, wholly or in part to the pathology associated with a particular disease or disorder. In another embodiment, the pathology is for example, is associated with other factors, for example ischemia and the like.

The term “biological sample” as used herein refers to a cell or population of cells or a quantity of tissue or fluid from a subject. Most often, the sample has been removed from a subject, but the term “biological sample” can also refer to cells or tissue analyzed in vivo, i.e. without removal from the subject. Often, a “biological sample” will contain cells from the animal, but the term can also refer to non-cellular biological material, such as non-cellular fractions of blood, saliva, or urine, that can be used to measure gene expression levels. Biological samples include, but are not limited to, whole blood, plasma, serum, urine, semen, saliva, aspirates, cell culture, or cerebrospinal fluid. Biological samples also include tissue biopsies, cell culture. A biological sample or tissue sample can refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tissue biopsies, scrapes (e.g. buccal scrapes), tumors, organs, and also samples of in vitro cell culture constituent. In some embodiments, where the sample is solid, it can be liquidized and homogenized into a liquid sample for use in the device and systems as disclosed herein. In some embodiments, the sample is from a resection, bronchoscopic biopsy, or core needle biopsy of a primary or metastatic tumor, or a cellblock from pleural fluid. In addition, fine needle aspirate samples are used. Samples may be either paraffin-embedded or frozen tissue. The sample can be obtained by removing a sample of cells from a subject, but can also be accomplished by using previously isolated cells (e.g. isolated by another person), or by performing the methods of the invention in vivo. Biological sample also refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the biological samples can be prepared, for example biological samples may be fresh, fixed, frozen, or embedded in paraffin.

The term “tissue” is intended to include intact cells, blood, blood preparations such as plasma and serum, bones, joints, muscles, smooth muscles, and organs.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, interdisposition, affection. A disease and disorder, includes but is not limited to any condition manifested as one or more physical and/or psychological symptoms for which treatment is desirable, and includes previously and newly identified diseases and other disorders.

The articles “a” and “an” are used herein to refer to one or to more than one (i.e., to at least one) of the grammatical object of the article. By way of example, “an element” means one element or more than one element.

Other than in the operating examples, or where otherwise indicated, all numbers expressing quantities of ingredients or reaction conditions used herein should be understood as modified in all instances by the term “about.” The term “about” when used in connection with percentages can mean±5%. The present invention is further explained in detail by the following examples, but the scope of the present invention should not be limited thereto.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### General

The present invention provides an integrated microfluidic device where directional fluid flow of a valveless microfluidic device is controlled using remote valve switching device, and changes in fluid flow rate and/or velocity can be controlled using flow changing fluid reservoirs present on the device.

### System

The inventors have demonstrated the fully functional microfluidic system, as shown in FIGS. 1 and 2A-2B, which comprises at least any of the following; a control unit for automated remote control of the fluid flow in the microfluidic chip, and a sample analysis or detection subsystem for end-sample analysis, such as an optical detection subsystem. In some embodiments, the sample analysis or detection subsystem e.g. an optical detection subsystem analyses the sample on-chip, and in alternative embodiments, the analysis is done of the end-product sample off chip. Alternatively, in some embodiments, the sample analysis/detection subsystem e.g. an optical detection subsystem can perform continuous analyses the sample on-chip. In some embodiments, microfluidic system further comprises a thermal interface which is controlled by a thermal controller for integrated temperature cycling on the microfluidic device. The interface of the fluid control system has the capability of accommodating various chip different designs, including any generic microfluidic chip, as well as microfluidic chips comprising reservoir buffers which allow sample processing through both high and low flow processes on a single integrated microfluidic chip.

Accordingly, provided herein is a system where a change in direction of fluid flow on a microfluidic chip is controlled off-chip using a remote valve switching system, and a change in rate of fluid flow on the microfluidic chip is controlled on-chip using reservoir buffers. This is a highly diversifiable system enables the remote control of fluid flowing through a disposable integrated microfluidic chip, both in terms of direction and fluid flow rate. In some embodiments, the remote valves are controlled by a control unit, and can be controlled in an automated manner to regulate the fluid flow on a disposable integrated microfluidic chip.

A system which enables the control of fluid direction has been demonstrated using remote valve system shown in FIGS. 1 and 2A with an integrated microfluidic chip shown in FIG. 5, which comprises two flow changing reservoirs to detect the presence of Bacillus subtilis in a biological sample. In shown herein in Example 2, the inventors demonstrate use of the system of FIGS. 1 and 2 for remote valve switching to control fluid flow in an integrated microfluidic chip of FIG. 5A. The chip of FIG. 5A comprises at least two reservoirs, at least two mixers, a nucleic acid extraction column (SPE column) and a PCR column and was used for integrated gene expression analysis on-chip using a TaqMan assay to detect the isolated bacterial DNA. As shown in FIGS. 5A and 5B, a microfluidic device comprising a flow changing reservoir comprises at least one or more components to conduct bacterial lysis, nucleic acid isolation (e.g. a SPE column), nucleic acid concentration and polymerase chain reaction (PCR) (e.g. a PCR channel). In some embodiments, a PCR product can be analyzed using end-point fluorescent detection using the sample analysis detection subsystem as shown, for example in FIG. 2A, where the detection is performed on the sample on-chip.

In particular, referring to FIG. 1, a system for remote-valve switching can be used control directional fluid flow on a microfluidic chip, where the valves of the remote valve-switching system are controlled by a control unit, e.g. a fluid control subsystem, and where each valve is fluidly connected to an input and/or output port at the interface of an integrated microfluidic chip, where the integrated microfluidic chip comprises at least one flow changing reservoir to change (e.g. increase/decrease) the flow rate and/or velocity between a high flow and low flow process (or vice versa). In one embodiment, the control unit is connected to a computer, where the control unit is connected to, and controls the opening and closing of each valve which is fluidly connected to an input and/or output port at the interface with the microfluidic device.

One can use any system to controlling the rate of fluid flow through the valves connected to an input and/or output port at the interface with the microfluidic device. For example, as shown in FIG. 2A, one can use syringe pumps, pneumatic dispensers and flow restrictors, which can direct fluid by air or other fluids.

In some embodiments, a valve at the interface is connected to a pump which is connected to a tank, where the pump pumps fluid from the tank at pre-determined flow rate to the valve at the interface. The valve connected to the pump can be used for low flow rate or high flow rate input into the microfluidic device. In some embodiments, a valve at the interface is connected to a flow restrictor. A tank is connected to both a valve and a flow restrictor, and in some embodiments, a pressure regulator (not shown), and in some embodiments, the pressure regulator (e.g. a pressure regulator) can establish a pre-determined pressure in the tank, and where pressure drives the fluid through a flow restrictor to the valve which is fluidly connected at the interface. The valve connected to flow restrictor can be used for low flow rate or preferably high flow rate flow input into the microfluidic device. In an alternative embodiment, a valve at the interface is connected to a tank which is connected to another valve, where fluid flows from tank to the valve which is fluidly connected at the interface. In some embodiments, a valve which interfaces with the microfluidic device is connected to air, for example, for input and/or output of air into the microfluidic device. In some embodiments, a valve at the interface is connected to an output port on the microfluidic device, where the valve is fluidly connected to a waste tank or chamber. In some embodiments, a valve at the interface is connected to an output port on the microfluidic device, where the valve is fluidly connected to an end sample chamber. In some embodiments, an end sample chamber is connected to a sample analysis detection subsystem, which is connected to the computer. In alternative embodiments, as disclosed earlier in reference to FIG. 1, the sample analysis detection system is connected to a detection/analysis interface, which connects with the microfluidic chip for sample detection on-chip, for example, continuous detection, such as, for example, real-time detection of the production of a product. In some embodiments, the control unit connects to a thermal controller, which is connected and controls the temperature of the thermal interface which interfaces with an integrated microfluidic chip. The thermal interface typically only interfaces with a region or part of the microfluidic chip which comprises PCR channels (see FIG. 5).

In some embodiments, the system can optionally comprise a sample analysis or detection system connected to a computer. In some embodiments, the sample analysis or detection system is an optical subsystem as shown in FIG. 2A. This is useful for analyzing the end product sample, either on chip (see FIG. 5), or alternatively a sample removed from the chip and collected in an end sample collection chamber. However, in alternative embodiments, the microfluidic chip comprising the end-product sample is removed from the system and analyzed the sample can be analyzed, either still present on the chip, or removed in a separate container, using a separate optical detection system. Such an embodiment is useful if the sample analysis, e.g. optical measurement is technically difficult or requires a significant quantity of time, or if a special apparatus or wavelength for sample analysis. Performing the sample analysis using an independent system would free up the remote-valve system of the present invention to enable increased samples to be processed and thus increases turn-over and sample processing efficiency. Furthermore, using an independent sample analysis is useful where the sample is to be removed from the microfluidic device and transferred to a separate container for analysis, for example, a multi-well plate (e.g. 96, 384-well plates), which allows sample storage (for future use, e.g. cloning or amplification), as well as enabling high-throughput sample analysis of multiple samples simultaneously. This is advantageous for comparing multiple samples at the same time for comparison and accuracy as well as optimization purposes, as well as reduces the time each microfluidic chip is in the system of the invention, thus increasing efficiency and throughput of the system.

In some embodiments, the system can optionally comprise a thermal control subsystem which is connected to a thermal interface with the microfluidic device. As discussed in Example 1, the thermal interface allows for controlled ramping of the chip temperature for a cycle time which enables PCR to be performed on the integrated microfluidic chip. The thermal interface typically contacts the microfluidic device at a region comprising PCR channels or other channels conducive to PCR of nucleic acids. In an alternative embodiment, the thermal interface controlled by a thermal controller (e.g. thermal control subsystem) can be used for non-PCR thermocycling, for example for thermal control of the microfluidic chip, e.g. isothermal amplification or for multiple heaters for continuous flow amplification. Where continuous flow PCR is desired, the thermal interface can comprise multiple temperature heating devices which are spatially distinct to allow for continuous flow PCR amplification.

The thermal interface can comprise a heating device, which can be selected to heat and cool a substrate, including an external resistive heater such as a nichrome coil; a Peltier device; flowing air, gas, water or other liquids past a device; rapidly moving a microchip from one thermal zone to another; IR heating; heating by alternating current; or other methods well known to function at the macroscale, microscale or smaller dimension. Usefully, feedback from a temperature measuring device such as a thermocouple and control software control the temperature regulation to thermally cycle or maintain an isothermal or other profile.

### Instrument

In some aspects of the present invention, the remote-valve switching device is located in a housing to which the valveless microfluidic device is placed into, as disclosed in the examples and in FIG. 2. In some embodiments, the instrument houses detection apparatus, such as fluorescent spectrometers and the like, which may be adapted to detect a desired readout of the on-chip microfluidic device procedure, such as detection of the presence of PCR product for gene expression analysis, such as the TaqMan procedure as disclosed herein in Example 1.

In some embodiments, the instrument houses at least one and any combination of the following components; a remote valve switching device, an actuator of the valves of the remote switching device, detection apparatus, apparatus or heating device to enable PCR thermal cycling, fluorescence detection, temperature sensors, pressure sensors, humidity sensors and the like.

In some embodiments, the instrument is also connected to a display module (FIG. 2B) which displays data which can be recorded or printed by the end user. In some embodiments, the instrument also is connected to a computer systems and other modules, including but not limited to a processing module, a control module for controlling a desired reaction, such as PCR on the microfluidic device, a database module, a user interface module, a storage module, a memory module, a network which can communicate to remote clients, a communication interface.

### Remote-Valve Switching Device

### Valves

Referring to FIG. 10, one can use any valve 380 for the remote-valve which is commonly known by one of ordinary skill in the art, for example a singular valve, a multiple valve or a moving valve operation. A valve can be any means to “plug” or function as a “liquid stopper” to inhibit flow of fluid through a channel of the remote-valve microfluidic device. The valves can be actuated manually, mechanically, electronically, pneumatically, magnetically, fluidically or by chemical means (e.g. hydrogels). The present invention is not limited to any particular type of valve. In some embodiments, passive or active valves can be used. Passive valves have been disclosed in U.S. Pat. No. 6,296,020 which is incorporated in its entirety herein by reference. In some embodiments, a valve 380 is a fluid impedence region which results in constriction of a channel or any other suitable means for impending flow of the fluid from the arm channel.

In some embodiments, the remove valves 380 can be controlled using control lines that control the opening and closing of the remote-valves. For example, in one embodiment, a remote valve can be controlled using macroscopic pressures that are located in the instrument, which are connected through control lines to the remote valves. In complex microfluidic devices with many remote-valve switching devices, electrical actuating devices can be used to open and close a mechanical remote-switching valve.

In some embodiments, the valve 380 in the system is a disposable valve, such as, for example a capillary stop, which is commonly known in the art and as the name suggests, prevent liquids from flowing through the capillary. If the capillary passageway is hydrophilic and promotes liquid flow, then a hydrophobic capillary stop can be used, i.e. a smaller passageway having hydrophobic walls. The liquid is not able to pass through the hydrophobic stop because the combination of the small size and the non-wettable walls results in a surface tension force which opposes the entry of the liquid. A hydrophobic valve has been disclosed in U.S. Pat. No. 6,296,020 herein incorporated in its entirety by reference.

In some embodiments, where the remote-valve 380 is a hydrophobic stop located in a hydrophilic capillary, a pressure difference must be applied to overcome the effect of the hydrophobic stop. In some embodiments, the pressure difference needed is a function of the surface tension of the liquid, the cosine of its contact angle with the hydrophilic capillary and the change in dimensions of the capillary. That is, a liquid having a high surface tension will require less force to overcome a hydrophobic stop than a liquid having a lower surface tension. A liquid which wets the walls of the hydrophilic capillary, i.e. it has a low contact angle, will require more force to overcome the hydrophobic stop than a liquid which has a higher contact angle. The smaller the hydrophobic channel, the greater the force which must be applied.

### Method of Controlling Fluid Flow Direction Using Remote Valves

One aspect of the present invention relates to a remove valve which controls the fluid direction of a microfluidic device. Referring now to the drawings, FIG. 10A shows an embodiment comprising two remote valve switching devices, which are input valves (V_{I}), where one in a closed configuration (bottom, V_{I2}) and one in an open configuration (top) (V_{I1}) in liquid communication with a channel on a microfluidic device. By way of an example, and referring to FIG. 10A, a remote valve microfluidic device can be used to control the direction of fluid flow in channels of the microfluidic device by preventing the flow along one arm of the channel, thus the fluid flow is directed along the channel as shown by the arrow in FIG. 10A.

The chip interfaces with the instrument by mating to an interface block, and in some embodiments, is sealed with a fluid impermeable material, for example, rubber o-rings, or other equivalent sealing mechanism at each of the fluid inputs and outputs. In alternative embodiments, the material at the interface junction between the microfluidic chip and the interface block can be any material commonly known to persons of ordinary skill in the art which prevents fluid leakage, for example, elastomer film and the like. The remote valve switching takes advantage of the inherent incompressibility of liquids such that once all the fluid lines are full, the fluids in the chip can be driven by remote pumps and switched by remote valves. For instance, suppose that a channel branches to two outputs, A and B. Both outputs connect to tubes which lead into the instrument, where they pass through valves A and B. To switch the flow out through A, the instrument opens valve A and closes valve B. As long as there is no compressibility in the entire path between the branching point and valve B, then no fluid can flow into channel B, and thus it will all flow through output A. This effectively works like a valve located at the branch point, but is far cheaper and easier to implement. In order for this design to work, the channel needs to be infinitely stiff to avoid a small amount of liquid being displaced in the ‘closed’ channel.

The microfluidic device 260 has a channel, and in some embodiments, the channel has a junction or branch point, such as a T junction as shown in FIGS. 10A and 10B, or a “Y” branch. In some embodiments, a remote switching valve comprises a substrate, such as a fluid impermeable substrate which can be configured to allow fluid flow through the valve (e.g. open configuration), or configured to prevent fluid flow through the valve (e.g. closed configuration) (see FIG. 10A). In other embodiments, the remote valves interface with the same external surface of the microfluidic device, which is advantageous to reduce manufacturing cost of the microfluidic devices (see FIG. 10C). If the valve is in its open position, fluid can flow uninterrupted from the remove valve to the microfluidic chip, (e.g. for an Inlet Valve (V_{I})), or vice versa (e.g. for an Exhaust Valve (V_{E})). When a remote valve is in a closed position, fluid flow is completely interrupted from the remove valve, blocking any fluid flow along the channel connected to the closed valve, therefore diverting fluid in a different direction on the microfluidic chip. In some embodiments, a fluid impearmeable material, such as an O-ring seal is located at the interface between the external surface of the microfluidic device and the external surface of the interface which is connected to the remote-valve to prevent fluid leakage at the interface.

As shown in FIG. 10A showing input valves (V_{I}), where one input remote valve is closed position, fluid cannot flow in the arm channel of the microfluidic device to which the remote valve channel is fluidly connected to, and thus the path of the fluid is directed or “pushed” along another arm channel which is fluidly connected to remote valve in an open configuration. In embodiments where both remote valves fluidly connected to the channels on the microfluidic device are in the closed configuration, fluid flow in the channel is stopped.

As shown in FIG. 10B showing output valves (V_{I}), where one output remote valve is closed position, fluid cannot flow in the arm channel of the microfluidic device to which is connected to the closed remote valve, and thus the path of the fluid is directed or “pushed” along another arm channel which is fluidly connected to an Exhaust remote valve (e.g. V_{E1}) in an open configuration.

One aspect of the present invention relates to a system for remotely controlling the direction of fluid flow in at least one channel on a microfluidic device, the system comprising: (i) at least one microfluidic device having at least one channel, said channel having at least one input and at least one output, wherein at least one input or at least one output are on an external surface of the microfluidic device; (ii) a plurality of remote-valve microfluidic devices, each containing a valve 380 and at least one channel bias towards and reversibly sealed to, at least one microfluidic chip, creating in interface therebetween; wherein the channel of each remote-valve microfluidic device is capable of fluidly communicating with an input or output of a channel on the microfluidic device across the interface; (iii) a means for opening and closing the valve in at least one remote-valve microfluidic device, wherein an open valve position allows fluid to flow across the interface between the channel of the remove valve microfluidic device and a channel or arm channel of the microfluidic device, whereas a closed valve position prevents the flow across the interface between the channel of the remove valve microfluidic device and a channel or arm channel of the microfluidic device. In some embodiments, the channel of the microfluidic device has an inlet, at least one junction and at least two arm channels coming off the junction, each arm channel having an outlet on the same or different external surface of the microfluidic device. In some embodiments, there is a fluid impermeable substrate or material, e.g. an O-ring, at the interface between the microfluidic device and interface connecting to the remote-valves.

In some embodiments, the system comprises the microfluidic device and the remote valves are housed in an instrument. The directional flow of fluid along channels on a microfluidic device can be controlled by any number of remote-valve switches or remote valve devices, for example, by at least one, at least 2, or at least 3, or at least 4, or a least 5, or at least 6, or at least 7, or more than 7, e.g. at least 10 or 10-15 or 15-25 remote valves fluidly connected to input/output ports of the microfluidic device. In some embodiments, a remote valve device can be used to control the input flow of fluid into an input of a channel of a microfluidic device, or alternatively, control the output flow of an output of a channel or arm channel of a microfluidic device. In some embodiments, the microfluidic device which is fluidly connected to at least one remote valve device is valveless (i.e. absent of any internal valves), however, in alternative embodiments a microfluidic device can comprise at least one valve which is controls the fluid flow of a channel which is not controlled by a remote valve device of the present invention.

Stated another way, in some embodiments the remote valve microfluidic device system functions as follows: fluid is loaded into the inlet of the microfluidic trunk channel and fills the arm channels and the fluidly connected channel of the remote valve device to the valve, and if the remote valve device is closed, the fluid in the arm channel functions as a liquid stopper, and fluid flow movement is directed along the arm channel connected to the remove valve which is in the open position (See FIGS. 10A and 10B). Because the arm channel has a volume, the liquid stopper has a discrete volume of the volume of the arm branch up to the branch point. Typically, a remote valve device or a set of remove valve devices are interconnectedly controlled, in come embodiments via actuators, to control the flow of fluid along each arm channels that branches off from a channel on a microfluidic device. Thus in some embodiments, one aspect of the present invention relates to use of multiple remote valve devices fluidly connected to each arm channel for the directional control of fluid flow in a network of branching channels in a microfluidic device.

### Microfluidic Chips Comprising Reservoirs for Increasing or Decreasing Velocity

One aspect of the present invention relates to a system and microfluidic device for changing the velocity on a microfluidic device and therefore enabling multiple fluid velocities on the same microfluidic device. Referring now to the drawings. FIG. 7 shows one embodiment of a microfluidic device comprising a the reservoir buffered mixer to decrease the velocity on the microfluidic device, where the reservoir 40 is positioned between a high flow chamber such as mixer 30 and a low flow chamber such as a PPM column 50. First, the system or microfluidic device is primed by loading, manually or on an automated basis using a pipette or similar apparatus with a holding solution 210 into the mixer 30 and reservoir 40 and the low flow chamber, such as a SPE or PPM column 50 via an inlet 1 160 or inlet 2 170. As shown in FIG. 8, when an exhaust valve 150 is in the closed position, and at least one sample is loaded into input 1 160 and/or input 2 170 at a desired velocity (i.e. a high velocity, i.e. a high velocity for optimal functioning of the high flow chamber (i.e. for mixing)) either manually or on an automated basis using a syringe or similar apparatus, the combined sample 220 flows through a capillary channel 180 into high flow chamber, such as a zig-zag mixer 30. In an embodiment where the exhaust valve 150 is in the open configuration, the sample 220 flows at the high velocity from the outlet of the high flow chamber 190 such as mixer 30 into the reservoir 40 via a first reservoir inlet 200 to displace the holding solution 210 in the reservoir 40 into the waste chamber (not shown). When the exhaust valve 150 is in the closed configuration, as shown in FIG. 9, a propulsion buffer 230 can be loaded at a desired velocity, for example the velocity for optimal functioning or efficiency of the high flow chamber into the reservoir 40 via the first reservoir inlet 200 the same way the sample 220 entered, or alternatively via a second reservoir inlet (not shown) to displace the sample 220 in the reservoir chamber 40 into a low flow chamber, such as a SPE or PPM column 50. In some embodiments, the flow rate of the sample through the high flow chamber, such as a mixer 40 is about 6-8 μL/sec or about 360-480 μL/min. In some embodiments, the flow rate of the sample through a low flow chamber, such as SPE or PMM 50 column is less than 1 μL/sec, for example, about 0.18 μL/sec or about 10.8 μL/min. Stated in velocity, in some embodiments, the velocity of the sample through the high flow chamber, such as a mixer 40 is about 150 mm/sec or about 100-200 mm/sec. In some embodiments, the velocity of a sample through a low flow chamber, such as SPE or PMM 50 column is less than about 150 mm/sec, for example, about 15-149 mm/sec or or about 75-149 mm/sec, or about 15-75 mm/sec, or about 15-50 mm/sec or between 1-50 mm-sec or less than 50 mm/sec.

In some embodiments, a microfluidic device can comprise at least one reservoir located between and in liquid communication with at least one high flow chamber and/or at least one low flow chamber for changing the fluid velocity multiple times on the same microfluidic device. As shown in the Examples and in FIG. 1, a first reservoir 40 can be located between a first high flow chamber, such as a mixer 30 and a low flow chamber such as a SPE column 50, and a second reservoir 100 can be located between the same SPE column 50 and a second high flow column, such as a mixer 110 to allow, for example the change of fluid velocity from high velocity to low velocity and to high velocity respectively.

In some embodiments, a microfluidic device may have for example at least 2 or at least 3 or at least 4 or more reservoirs according to the invention as disclosed herein, wherein the reservoir is located between alternating high flow chambers and low flow chambers. In some embodiments, a reservoir can be located between two low flow chambers where each low flow chamber requires a different low velocity for optimal functioning. In some embodiments, a reservoir can be located between two high flow chambers where each high flow chamber requires a different high velocity for optimal functioning.

In some embodiments, the sample 220 is pushed through the high flow chamber via the inlet using a syringe pump or other pneumatic dispenser.

Another aspect of the present invention relates to a microfluidic device comprising, (a) a reservoir 40 with an first input 200 and first output 230, the first input 200 being in liquid communication through a capillary passageway to the output 190 of a first chamber 30, (b) a first chamber 30 with at least one input 160, 170 and an output 190, the output being in liquid communication through a capillary passageway to the first input 200 of the reservoir; (c) a second chamber 50 with an input 240 and output 250, the input 240 being in liquid communication through a capillary passageway to the first output 230 of the reservoir 40. In some embodiments of this aspect of the present invention, the microfluidic device can further comprise a waste chamber and a waste valve, wherein the inlet of the waste chamber is in liquid communication through a capillary passageway to a second output on the reservoir and wherein the waste valve controls flow of fluid from the reservoir second output into the inlet of the waste chamber, wherein when the waste valve is in the open position, fluid flows from the reservoir second output into the inlet of the waste chamber. In a further embodiment, the microfluidic device of can further comprise a second input on the reservoir chamber, wherein the second inlet of the reservoir chamber receives a liquid to displace the liquid in the reservoir via the first output into the second chamber or via the second output into the waste chamber.

Reservoirs

A reservoir chamber for use in the microfluidic devices instruments and systems as disclosed herein may be various shapes, but typically they will be generally rectangular, circular or square in cross-section. In some embodiments, a reservoir chamber can comprise internal features such as steps or ramps, which are believed to have a minor effect on mixing of the liquids, although they may be included for other reasons. It is considered important that a reservoir chamber has sufficient space for the volume of liquid of the microchip procedure, i.e. of sufficient volume or capacity to receive the liquid from the inlet (i.e. from the element subsequent to it, such as a mixer) or to provide sufficient volume for the element in the next step, (i.e. the next element such as a SPE column) which ever is the larger volume.

In some embodiment, a reservoir is shaped as a thin channel, so that fluids can wet the reservoir completely without bubbles, and so the propulsion can displace the reservoir contents efficiently. In some embodiments, a reservoir is an interconnecting channel between a low flow chamber and a high flow chamber. Embodiments where the where the sample volume is small enough to fit in the normal channel cross-section/length.

In some embodiments the reservoir is in a coiled configuration to conserve space on the microfluidic device. In some embodiments, the reservoir chamber is about 0.020″ wide and about 0.020″ deep. The volume of the reservoir is dependent on the volume and length of the reservoir chamber is dependent on the volume of liquid required for the subsequent or proceeding step on the microfluidic device. In some embodiments, a reservoir chamber of about 0.020″ wide and 0.020″ deep has a length of about 3-15″ long, for example, in the range of 5-10″ long, or for example, about 3″, or about 4″, or about 5″, or about 6″, or about 7″, or about 8″, or about 9″, or about 10″, or about 11″, or about 12″, or about 13″ long etc. As stated previously, in some embodiments, the length of the reservoir is dependent on the volume required for proceeding and subsequent steps on the microfluidic device. In some embodiments, a microfluidic device can be coiled into an arbitrary shape, for example a serpentine path to conserve space on the microfluidic device.

High Flow Chamber

One aspect of the present invention relates to a reservoir between a high flow chamber or element, and a low-flow chamber or element on a microfluidic device.

In some embodiments, a high flow element is any element or capillary on a microfluidic device which as a flow volume per minute of 2 or 3 or 4 or 5 to 10-times or between 10-100 times faster, or more that the flow rate of a low flow chamber as that term is defined herein. In some embodiments, a high flow chamber is any element or capillary on a microfluidic device which as a flow distance per second of 2 or 3 or 4 or 5 to 10-times or between 10-100 times faster, or more that the velocity of a low flow chamber as that term is defined herein. In some embodiments, a flow rate of a high flow chamber is about 6-8 μL/sec or 480 μL/min, or any rate above about 200 μL/min or any rate above about 500 μL/min or any rate above about 1 mL/min or any rate above 1 mL/min, for example, at least 1.2 mL/min, at least 1.5 mL/min, at least 2 mL/min above. In some embodiment, the velocity of a high flow chamber is greater than about 155 mm/sec, and can be any range from 150 mm/sec to 1500 mm/second or greater than 1500 mm/second. In some embodiments, a velocity of fluid through a high flow chamber has a velocity of greater than 150 mm/sec or any rate above 150 mm/sec, for example, at least about 200 mm/sec, or at least about 300 mm/sec, or at least about 400 mm/sec, or at least about 500 mm/sec, at least 600 mm/sec, or at least 800 mm/sec mL/min, or at least about 1000 mm/sec, or at least about 1200 mm/sec, or at least about 1500 mm/sec or above.

In some embodiments of all aspects of the present invention, a high flow element is a mixer column designed to mix at least one fluid with a second or more fluid. In other embodiments, a high flow column is any high flow element on a microfluidic device, including mixers or lysis columns and the like. In some embodiments, a mixer element can be in any confirmation of a mixer element which requires a high flow, such as zig-zag configuration, coanda effect channels, v-shaped bas-relief shaped mixers, other micromixer configurations such as slanted groove, staggered herringbone and herringbone mixers and other mixers known in the art. In some embodiments, a zig-zag mixer high flow element is used, wherein each leg of the zig-zag mixer is about a 60-degree angle with respect to the next leg, and the total number of legs is sufficient for complete mixing. In some embodiments, a total number of legs is about 30-50 legs, for example 48 legs as shown in FIG. 6. Each leg is about 0.007″ deep, 0.007″ wide and about 0.050″ long.

In some embodiments of the present invention, complete mixing is desired. It has been found that through mixing can be achieved using alternative designs other than the zig-zag mixer as disclosed herein, and any design known or developed by one of ordinary skill in the art which results in uniform mixtures are encompassed as a mixer in the present invention, and can be used to combine liquid samples with liquid reagents or conditioning agents that have differing viscosities and volumes.

Any chamber which allows adequate mixing of more than one liquid is encompassed as a mixing chamber as that term is described herein, In some embodiments, mixing chambers are commonly known mixing chambers, such as those disclosed in U.S. Pat. No. 7,347,617 which is incorporated in its entirety herein by reference, or modification of mixing chambers depending on the viscosity and relative volumes of the liquids being mixed. It will be evident that mixing a viscous liquid with one that is much less viscous will be more difficult than mixing two liquids having similar low viscosities. Mixing two viscous liquids also will be difficult to do uniformly. Combining two liquids having significantly different volumes would be expected to be more difficult than mixing liquids of equal volumes.

In general, two or more liquids are combined in a first chamber, which is emptied through at least one connecting capillary “mixing” passageway into the reservoir chamber as disclosed herein. Movement of the liquids typically requires application of pressure to overcome the resistance to flow inherent in the use of small passageways. In some embodiments, pressure can be applied, for example using any one of the following; air pressure, vacuum, electroosmosis, absorbent materials, additional capillarity, mechanical displacement such as by a syringe pump, and the like. The force applied is sufficient to create a flow of liquid in the capillary passageways of 1 mm/sec or more. In some embodiments, the mixing passageways have cross-sectional dimensions between 1 μm and 2000 μm, preferably 200 to 1000 μm, as determined by the physical properties of the liquids. The passageways will have a length between 0.5 and 100 mm, preferably 1-50 mm, depending on the arrangement of chambers and passageways on the chip.

As mixing is usually done by creating chaotic conditions (e.g. chaotic advection) or in some embodiments, with turbulent conditions, it is typically done at high velocities and thus any mixing element known by a person of ordinary skill in the art which requires a high velocity is encompassed in the present invention. In some embodiments, a high velocity element is an active micro mixer. For example, U.S. Published Patent Application 2002/0097532 which is incorporated herein in its entirety by reference disclosed a disc containing many channels. Two liquids were passed through a zig-zag channel in laminar flow while the disc was rotated, with mixing said to occur by diffusion. In U.S. Published Application 2001/0048900 (herein incorporated in its entirety by reference), mixing separate streams by creating a vortex in a chamber. In some embodiments, the inventors indicate that a Reynolds number of 320 is achieved and the first and second fluids have Reynolds numbers between 1 and 2000. Therefore, the flow is in a region between laminar flow and turbulent flow.

High flow mixers are disclosed in US Application 2008/0043570 which is incorporated herein by reference. U.S. Pat. No. 5,921,678 (herein incorporated in its entirety by reference) discloses a liquid mixer in which two streams of liquid meet head-on and exit together in a channel 90 degree from the entrance channels. The Reynolds number of the streams is said to be 2000-6000. Sharp-edged pillars are shown to assist in generating turbulence at the intersection of the mixing streams.

U.S. Published Application 2002/0048535 (herein incorporated in its entirety by reference) shows a device in which two liquids are combined during rotation of the device to transfer the liquids from one container to another. U.S. Pat. No. 6,264,900 (herein incorporated in its entirety by reference) provides mixing of parallel laminar flow streams for carrying out fast chemical reactions. U.S. Pat. No. 6,065,864 (herein incorporated in its entirety by reference) discloses a micro-mixing system including bubble-controlled pumps and valves to establish circulating flow in a mixing chamber.

Low Flow Element or Low Flow Chamber

A low flow chamber is any chamber or element on a microfluidic device which requires a low velocity to function (e.g. binding events) or which has very high flow resistance and thus a low velocity. Typically a low flow chamber with a high flow resistance will require about 100 psi of pressure to generate a flow rate of about 1 μL/sec. In some embodiments, a low flow element is any element or capillary on a microfludic device which as a flow volume per minute of 2 or 3 or 4 or 5 to 10 times slower, or 10-100 times slower that the flow rate of a high flow element as that term is defined herein. In some embodiments, a velocity of a low flow element is a velocity of less than 200 μL/min velocity or any rate below, for example, in the range of 0-199.9 μL/min, or for example within the range of 0.1 μL/min to 10 μL/min or 1.0 μL/min to 50 μL/min, or in the range of 50 μL-100 μL/min or in the range of 100-199.9 μL/min or any range therebetween. In some embodiments, the velocity of low flow chamber is for example, about 0.18 μL/sec or about 10.8 μL/min.

In some embodiments, a low flow chamber has a velocity of less than 155 mm/sec, and can be any range from 0.0001 mm/sec to 149.99 mm/second. In some embodiments, fluid flow through a low flow chamber has a velocity of between 15-150 mm/second. Typically a low flow chamber is any element or capillary on a microfludic device which as a flow rate which as a flow distance per second of at least 2 or 3 or 4 or 5 to 10 times slower, or 10-100 times slower that the flow rate of a high flow element as that term is defined herein.

In some embodiments of this aspect of the present invention, a low flow chamber is a solid-phase extraction (SPE) column which is commonly known by a skilled artisan and can be used to isolate the nucleic acids from the lysed cell, for example the lysed bacterial cell. In some embodiments, the solid-phase extraction column comprises a silica bead and polymer composite. In alternative embodiments, any solid-phase extraction column is useful in the methods of the present intervention, and such solid-phase extraction columns and nucleic acid extraction methods are commonly known by persons of ordinary skill in the art and are encompassed for use in the present invention. For example but not limited to, the following examples are useful for nucleic acid extraction according to the methods of the present invention; silica bead packed solid phase extraction column, silica membranes, high surface area pillar chip modules, Leukosorb filters and Nano-gap channel arrays.

Microfluidic Devices

The analytical devices of the invention may be referred to as “chips”. They are generally small and flat, typically about 1 to 2 inches square (25 to 50 mm square) or disks having a radius of about 40 to 80 mm. The volume of samples will be small. For example, they will contain only about 0.1 to 10 μL for each assay, although the total volume of a specimen may range from 10 μL to about 500 μL, for example, about 250 μL-350 μL, or about 350 μL or about 300 μL-400 μL or greater than 400 μL, such as 500 μL or greater than 500 μL. The chambers holding the sample fluids and reagents typically will be relatively wide and shallow in order that the samples can be easily seen and changes resulting from reaction of the samples can be measured by suitable equipment. The interconnecting capillary passageways typically will have a cross-sectional dimension in the range of 1 to 2000 μm, preferably 200 to 500 μm. The shape will be determined by the method used to form the passageways but passageways having rectangular cross-sections are preferred. The depth of the passageways will be at least 5 μm in many practical applications where samples contain particles, but may be smaller where the nature of the sample permits.

While there are several ways in which the capillaries and chambers can be formed om hard plastics such as injection molding, laser ablation, diamond milling or embossing, it is preferred to use injection molding in order to reduce the cost of the chips. Generally, a base portion of the chip will contain the desired network of chambers and capillaries. After reagent compounds have been placed in the chambers as desired, a top portion “cover slip” is attached over the base to complete the chip.

The chips usually are intended to be disposable after a single use. Consequently, they will be made of inexpensive materials to the extent possible, while being compatible with the reagents and the samples which are to be analyzed. In most instances, the chips will be made of plastics such as polycarbonate, polystyrene, polyacrylates, or polyurethene, alternatively, they can be made from silicates, glass, wax or metal.

Types of Thermoplastic Materials for Substrates of the Microfluidic Devices and Remote-Valve Microfluidic Devices

In some embodiments, the microfluidic chips as disclosed herein are made of plastic, and as such will be much cheaper than other microfluidic chips available in market which are made of glass or quartz.

Most currently available microfluidic devices are made of silicon and/or glass. Use of silicon and glass is relatively expensive because of high material and manufacturing costs. Polymeric materials would be less expensive. Therefore, microfluidic devices made from polymeric materials are more suitable for mass-production of disposable devices. In one embodiment, the microfluidic devices disclosed herein are made using cyclic polyolefin, such as ZEONEX® (ZEONEX 690R, Zeon Chemicals Inc. Louisville, Ky., USA). For example, the inventors demonstrated herein that the mechanical and optical properties of cyclic polyolefins, such as ZEONEX are suitable for chip manufacturer.

In some embodiments, the microfluidic device disclosed herein is made of thermoplastic polymer that includes a channel or a multiplicity of channels whose surfaces can be modified by photografting. In some embodiments, the device further includes a porous polymer monolith (PPM) impregnated with silica particles or carbon particles for cell lysis of a biological sample, as disclosed in International Patent Application WO2009/002580 (herein incorporated in its entirety by reference), where the PPM impregnated with silica or carbon particles can be prepared via UV initiated polymerization of a porous polymer solution embedded with the silica or carbon nanoparticles, within the channel.

In some embodiments, the microfluidic substrate is made with cyclic polyolefins as the chip material. In one embodiment, the inventors demonstrated use of ZEONOR® or ZEONEX® (Zeon Chemicals, Louisville, Ky., USA), medical grade cyclic polyolefins, to manufacture a plastic microfluidic device. The inventors used ZEONEX® the primary chip material, because of its excellent mechanical properties, low auto-fluorescence and high UV transmission. However, one can use any other material with suitable optical properties can be used. The optical properties necessary for both photoinitiated polymerization during manufacturing and the integration of on-chip detection in the future include good mechanical properties, low auto-fluorescence and high UV transmission.

In one embodiment, one forms the microchannels by hot embossing with a master at about 166° C. (about 30° C. above the Tg of ZEONEX or ZEONOR) and about 250 psi for about 2-5 minutes using, for example, a hot press, such as Heated Press 4386, Carver, Wabash, Ind. The master and the substrate can be manually separated at the de-embossing temperature, 60° C. Aluminum (Al) coating on the master facilitates easier removal of the master from the substrate after the embossing is completed. To seal the channels, another piece of ZEONEX or ZEONOR of the same dimensions can be thermally bonded on top, for example using 134° C., 6751b per sq inch of chip for 2 minutes.

In an alternative embodiment, one can prepare the microfluidic device as disclosed herein by hot embossing using wire embedded in the base plate of ZEONEX or ZEONOR substrate or by using a SU-8 master. Channels of about 100 μm and about 165 μm depths can be fabricated by this method. The width of the channels can vary from about 2 μm to at least about 500 μm. The width of the channels preferably vary from about 50 μm to about 250 μm or any width between, such as about 51 μm, or about 52 μm, or about 53 μm, about 54 μm, or about 55 μm, or about 60 μm, or about 65 μm, or about 70 μm, or about 75 μm, or about 80 μm, or about 85 μm, or about 90 μm, or about 100 μm, or about 115 μm, or about 125 μm, or about 150 μm, or about 200 μm, or about 249 μm. One can drill wells of any depth. In one preferred embodiment, one drills wells of about 1.5 mm diameter at the end of the channels for sample introduction and collection.

In some embodiments, where SU-8 master is used in fabrication of the device, the SU-8 masters can be fabricated, for example, on piranha-cleaned silicon wafers by spinning SU-850 photoepoxy (Microchem, Newton, Mass.) or any other comparable method. In one preferred embodiment, one uses thickness of about 100 μm and about 165 μm onto the wafers. One then pre-bakes the wafers as is known to one skilled in the art. For example, in one preferred embodiment, one pre-baked the wafers for 30 min at 95° C. After baking, the pattern is transferred through a mask preferably, by using contact lithography. Other applicable methods may be used as is known to one skilled in the art. One follows the transfer of the pattern by development, for example with SU-8 developer (Microchem) and post-baking the wafers for, for example, 1.5 h at 175° C. In one embodiment, after the fabrication process, the SU-8 molds exhibit glass-like mechanical properties and have the negative pattern of the microfluidic channels.

In some embodiments, the wafers are sputter coated with about 500 Angstroms (Å) of titanium (Ti) for adhesion, followed by about 100 Å of Al.

In another embodiment, one forms the microchannels by hot embossing with a master at about 100° C. (about 30° C. above the Tg of ZEONEX or ZEONOR) and about 250 psi for about minutes using, for example, a hot press, such as Heated Press 4386, Carver, Wabash, Ind. The master with and the substrate can be manually separated at the de-embossing temperature, 60° C. Aluminum (Al) coating on the master facilitates easier removal of the master from the substrate after the embossing is completed. To seal the channels, another piece of ZEONEX or ZEONOR of the same dimensions can be thermally bonded on top, for example using 134° C., 6751b per sq inch of chip for 2 minutes.

The capillary passageways are configured to be either hydrophobic or hydrophilic, properties which are defined with respect to the contact angle formed at a solid surface by a liquid sample or reagent. Typically, a surface is considered hydrophilic if the contact angle of water on the surface is less than 90° and hydrophobic if the contact angle is greater than 90°. Preferably, the surface energy is adjusted by plasma induced polymerization at the surface of the passageways. The analytical devices of the invention may also be made with other methods used to control the surface energy of the capillary walls, such as coating with hydrophilic or hydrophobic materials, grafting, or corona treatments. The surface energy of the capillary walls may be adjusted for use with the intended sample fluid. For example, to prevent deposits on the walls of a hydrophobic passageway or to assure that none of the liquid is left in a passageway. For most passageways in the microfluidic devices of the invention, the surface is generally hydrophilic since the liquid tends to wet the surface and the surface tension forces causes the liquid to flow in the passageway. For example, the surface energy of capillary passageways is adjusted so that the contact angle of water on the surface is between 10° to 60° when the passageway is to contact whole blood or a contact angle of water on the surface of 25° to 80° when the passageway is to contact urine.

Microfluidic devices can take many forms as needed for the analytical procedures which measure the analyte of interest. The microfluidic devices typically employ a system of capillary passageways connecting chambers containing dry or liquid reagents or conditioning materials. Analytical procedures may include preparation of a metered sample by diluting the sample, pre-reacting the analyte to ready it for subsequent reactions, removing interfering components, mixing reagents, lysing cells, capturing bio molecules, carrying out enzymatic reactions or incubating for binding events, staining, or deposition. Such preparatory steps may be carried out before or during metering of the sample, or after metering but before carrying out reactions which provide a measure of the analyte.

In such analytical procedures a sample will be combined with a conditioning liquid or with a reagent liquid and then transferred to a mixing chamber before being sent to subsequent processing. It will be evident that intimate mixing of the sample with the reagent or conditioning liquid is important to accurate and reproducible results. As is well known, the flow in microfluidic devices is typically laminar, that is, the viscosity of the liquid has a greater effect than the inertia of the flowing liquid so that the liquid flows linearly without being turbulent. One consequence of laminar flow conditions is that mixing of two or more liquids is slow since it principally results from molecular diffusion. As discussed above, some microfluidic devices have been designed to improve diffusion between layers of liquids in laminar flow. Many of these devices do not intend that complete mixing occurs, but in others provision for close contacting of liquid streams is provided.

The photografting method used in preparing the microfluidic chips of the present invention can be used for the surface modification of a wide range of thermoplastic polymers. The preferred substrates, i.e. for forming channel or tube surfaces, are selected from the group consisting of poly(methyl methacrylate), poly(butyl methacrylate), poly(dimethylsiloxane), poly(ethylene terephthalate), poly(butylene terephthalate), hydrogenated polystyrene, polyolefins such as, cyclic olefin copolymer, polyethylene, polypropylene, and polyimide. Polycarbonates and polystyrenes may not be transparent enough for efficient UV transmission and therefore may not be suitable for use as substrates.

Optical properties such as light transparency at the desired wavelength range and low background fluorescence are important characteristics of substrate materials that show potential for use in the microfluidic devices as disclosed herein. Since the photografting reactions must occur within the channels on all sides, the light must first pass through a layer of this polymer. Therefore, the substrate materials should be transparent in a wavelength range of about 200 to about 450 nm, preferably at any point in the range between about 330-380 nm such as about 350 to about 365 nm, or about 350 to about 395, etc.

In addition, the chemical properties and solubility of substrates can be taken into consideration. For instance, substrates that dissolve only in solvents, such as toluene and hexane, that are less likely to be used in standard microfluidic applications, make more desirable candidate substrate materials for photografting.

One important consideration in choosing substrate material for grafting is the grafting efficiency, expressed as Neff, of the monomer to the substrate, which depends on properties such as the chemistry and transparency for light at the desired wavelength range. Grafting efficiency values of substrates correlate well with the irradiation power, the measured values of contact angles and the transparency of the substrate. An opaque substrate with a grafting efficiency value of 0 would reflect a sample, wherein no transmitted light can be detected using the material as a filter and no grafting is achieved even after 30 minutes of irradiation.

Thickness of only a few micrometers of a UV absorbing material or solution could decrease the intensity of the UV light and, consequently, the grafting efficiency. The depth of features in typical microfluidic devices may reach several tens of micrometers. Therefore, it is important to assess the effect of UV transparency of the grafting monomer mixtures during the grafting more exactly in order to determine the depth of the channel through which sufficient grafting can be safely achieved with the chosen monomer mixture.

In general, the channel depth should be about 10-500 μm, preferably any range between about 10-250 μm including about 50-250 μm, most preferably about 10-50 μm. In some embodiments, when a channel is filled with PMM (e.g. for silica embedded PMM for a low-flow chamber for nucleic extraction), the channel depth is about 1 mm wide/1 mm thick. The thickness or width of the channel can be varied depending on the biomolecule one is looking at. For example, from about 35 μm to about 300 μm, and all ranges in between. In some embodiments, the channel ranges from about 50 μm to about 250 μm. In some embodiments, a channel can be about 100 μm depth and between about 100 μm and about 15 μm in width.

In some embodiments, wells can be prepared to introduce and collect samples at the ends of the channels. These can range from about 0.5 mm to about 2.0 mm, and all ranges in between, such as about 1.5 mm.

Other plastics can be used for the microfluidic devices, in particular the substrate for he remote-valve microfluidic device using for example, a variety of commercially available materials known by a skilled artisan such as, for example, polymethyl-methacrylate (PMMA), polystyrene (PS), polycarbonate (PC), polypropylene (PP), or polyvinylchloride (PVC). Other representative materials that can be used to fabricate upper and lower substrates 21, 22 include, but are not limited to polychlorotrifluoroethylene (PCTFE), polyetheretherketone (PEEK), polyetherimide (PEI), polyethersulfone (PES), polyethylene—carbon filled (PE), polyethylene—high density (HDPE), polyethylene—low Density (LDPE), polyethylene—U.H.M.W. (UHMW PE), polyethylene naphthalate (PEN), polyethylene terephthalate (polyester, PET, PETP), polyethylene/polyethylene composite (PE fibre—PE matrix), polyhydroxybutyrate—biopolymer (PHB), polyhydroxybutyrate/polyhydroxyvalerate 8%—biopolymer (PHB92/PHV 8), polyhydroxybutyrate/polyhydroxyvalerate 12%—biopolymer (PHB88/PHV12), polyimide (PI), polymethylpentene (TPX.RTM.), polyoxymethylene—copolymer (acetal—copolymer POMC), polyoxymethylene—homopolymer (acetal—homopolymer POMH), polyoxymethylene/acetal copolymer—10% carbon fiber reinforced (POMC-10% CFR), polyphenyleneoxide (PPO modified, PPE modified), polyphenyleneoxide (modified), polyphenylenesulfide (PPS), polyphenylenesulfide—40% glass fiber reinforced (PPS-40% GFR), polyphenylenesulphide—20% carbon fiber reinforced (PPS-20% CFR), polyphenylsulfone (PPSu), polypropylene (PP), polypropylene—polypropylene composite (PP fibre—PP matrix), polystyrene (PS), polystyrene—conductive (High Impact Conductive Polystyrene), polystyrene—cross-linked (PS—X-Linked), polystyrol, polysulphone (PSu), polytetrafluoroethylene (PTFE), polytetrafluoroethylene coated Glass Fabric (PTFE 75/Glass 25), polytetrafluoroethylene filled with glass (PTFE 25% GF), polyvinylchloride—unplasticized (UPVC), polyvinylfluoride (PVF), polyvinylidenechloride (PVDC), and polyvinylidenefluoride (PVDF). See, for example, product catalogs offered by Goodfellow Cambridge Limited, Huntingdon, Cambridgeshire, England. In the case of optical characterization, the substrate is preferably constructed out of a transparent plastic material.

Capillaries, reaction chambers, and pump chambers can be formed in substrates using methods such as injection molding, compression molding, hot embossing, machining, micro-compression molding, electrodischarge machining, injection compression molding, hot stamping, and micro injection molding. Methods for forming the features in the microfluidic devices include die cutting, die forging, blow molding, rotary die cutting, laser etching, injection molding, and reaction injection molding.

### Uses & Applications of Microfluidic Devices Comprising Flow Changing Reservoirs Controlled by Remote Valve Switching

Modular microfluidics should have widespread applications in diagnostics for biodefense, infectious diseases, forensics, genomics, and proteomics. The present invention of microfluidic devices, remote-valve switching devices and instruments can be integrated to provide a core technology to enable production of compact autonomous integrated microfluidic devices and instruments as disclosed herein with small footprints that can be deployed to the field for biodefense, disease and pathology epidemiology applications, for example, such as pathogen monitoring devices for buildings, planes, or airports, or at site locations and point-of-care clinics, as well as a laboratory version to cope with surges in testing demand. The microfluidic devices, systems and instruments as disclosed herein can prepare and analyze sample from air, biological fluids, agricultural products, or other matrices to detect target a pathogen. The combination of low consumable costs with automated preparation and analysis are likely to be extremely advantageous for rapid, and effective molecular diagnostics and have significant impact on the field of diagnostics.

In some embodiments, the microfluidic devices, such as the valveless microfluidic devices where fluid direction is controlled by remote-valve switching devices, and/or microfluidic devices comprising reservoir-buffered mixers, can be used for biological assays commonly known by one of ordinary skill in the art. For example, the remote-valve switching device, and/or a reservoir-buffered mixer can be used in microfluidic devices for detecting the presence of a pathogen in a biological sample, as disclosed in the examples.

Alternatively, microfluidic devices have many applications. Analyses may be carried out on samples of many biological fluids, including but not limited to blood, urine, water, saliva, spinal fluid, intestinal fluid, food, and blood plasma. Blood and urine are of particular interest. A sample of the fluid to be tested is deposited in the sample well and subsequently measured in one or more metering capillaries or wells into the amount to be analyzed. The metered sample will be assayed for the analyte of interest, including for example a protein, a cell, a small organic molecule, or a metal. Examples of such proteins include albumin, HbAlc, protease, protease inhibitor, CRP, esterase and BNP. Cells which may be analyzed include E. coli, pseudomonas, white blood cells, red blood cells, h.pylori, strep a, chlamdia, and mononucleosis. Metals which may be detected include iron, manganese, sodium, potassium, lithium, calcium, and magnesium.

Applications for the microfluidic devices as disclosed herein and integrated fluidic systems, apparatus and methods of the present invention include, but are not limited to, the areas of genomics, proteomics, molecular diagnostics and cell based assays. Examples, some of which are further described herein, include sample cleanup and purification, PCR, cycle sequencing, sample dilution, sample concentration, and isothermal, enzyme or ligand binding assays. Multiple reaction steps may be performed. Samples may be prepared for detection by mass spectrometry. In addition, applications exist in fields outside of life sciences.

The modular microfluidic devices, remote valve switch devices, integrated fluidic systems, apparatus and methods as disclosed herein are capable of implementing a wide range of applications, chemistries, biochemistries, processes, and analyses. The following examples are just some of the wide range of applications that can be implemented on modular microfluidic microchips. It is important to note that the sample preparation on modular microfluidic devices, systems and instruments as disclosed herein can be for on-chip analysis or moved off-chip for analysis with different instrumentation such as CAE, mass spectroscopy, microarrays, optical or other analytical methods. The examples are not meant to limit the invention scope but to illustrate specific examples of how modular microfluidic devices, systems and instruments as disclosed herein can be applied to develop genomic, proteomic, and metabolic assays.

In many applications, color developed by the reaction of reagents with a sample is measured. Other spectroscopic analysis of the sample are possible, using sensors positioned for detecting absorbance, reflectance, transmission and emission such as fluorescence, phosphorescence, luminescence, and other changes in the near and far infrared, Raman, and ultraviolet wavelengths. It is also feasible to make electrical measurements of the sample, using electrodes positioned in the small wells in the device. Examples of such analyses include electrochemical signal transducers based on amperometric, impedimetric, potentimetric detection methods. Examples include the detection of oxidative and reductive chemistries and the detection of binding events.

There are various reagent methods which could be used in microfluidic devices. Reagents undergo changes whereby the intensity of the signal generated is proportional to the concentration of the analyte measured in the clinical specimen. These reagents contain indicator dyes, metals, enzymes, polymers, antibodies, electrochemically reactive ingredients and various other chemicals dried onto carriers. Carriers often used are papers, membranes or polymers with various sample uptake and transport properties. They can be introduced into the reagent chambers in the microfluidic devices.

A number of uses for reagents are possible. For example, an analyte can be reacted with reagent in a first chamber and then the reacted reagent directed to a second chamber for further reaction. Also, a reagent can be re-suspended in a liquid in a first chamber and moved to a second chamber for a reaction. An analyte or reagent can be trapped in a first or second chamber and a determination of free versus bound reagent be made. A third liquid reagent can be used to wash materials trapped in the second chamber and to move materials to the waste chamber.

The determination of a free versus bound reagent is particularly useful for multizone immunoassay and nucleic acid assays. There are various types of multizone immunoassays that could be adapted to these devices. In the case of adaption of immunochromatography assays, reagents and filters are placed into separate chambers and do not have to be in physical contact as chromatographic forces are not in play. Immunoassays or DNA assay can be developed for detection of bacteria such as Gram negative species (e.g. E. Coli, Entereobacter, Pseudomonas, Klebsiella) and Gram positive species (e.g. Staphylococcus aureus, Entereococcus). Immunoassays can be developed for complete panels of proteins and peptides such as albumin, hemoglobin, myoglobulin, .alpha.-1-microglobulin, immunoglobulins, enzymes, glycoproteins, protease inhibitors, drugs and cytokines. See, for examples: Greenquist in U.S. Pat. No. 4,806,311, Multizone analytical Element Having Labeled Reagent Concentration Zone, Feb. 21, 1989, Liotta in U.S. Pat. No. 4,446,232, Enzyme Immunoassay with Two-Zoned Device Having Bound Antigens, May 1, 1984.

Potential applications where dried reagents are resolubilized include, filtration, sedimentation analysis, cell lysis, cell sorting (mass differences) and centrifugal separation. Enrichment (concentration) of sample analyte on a solid phase (e.g. microbeads) can be used to improved sensitivity. The enriched microbeads could be separated by continuous centrifugation or by filter methods. Multiplexing can be used (e.g. metering of a variety of reagent chambers in parallel and/or in sequence) allowing multiple channels, each producing a defined discrete result. Multiplexing can be done by a capillary array comprising a multiplicity of metering capillary loops, and fluidly connected with the entry port, or an array of dosing channels and/or capillary stops connected to each of the metering capillary loops. Combination with secondary forces such as magnetic forces can be used in the device design. Particle such as magnetic beads used as a carrier for reagents or for capturing of sample constituents such as analytes or interfering substances. Separation of particles by physical properties such as density (analog to split fractionation).

Microfluidic devices have also been used in assays to measure glycated hemoglobin (HbAlc) content of a patient's blood which can indicate the condition of diabetic patients. The method used has been the subject of a number of patents, most recently U.S. Pat. Nos. 6,043,043 and 7,347,617, which are both incorporated herein in their entirety by reference.

PCR Analysis on Modular Microchips

As demonstrated herein in Example 1 and 2, and using the microfluidic chip shown in FIG. 5, PCR can be readily adapted to modular microfluidic devices, systems and instruments as disclosed herein of the present invention. As adapted to the fluidic microchips, systems, methods, and apparatus of the present invention, PCR can provide evidence for the presence of pathogens as well as quantify the number of organisms, such as viruses, by RT-PCR, provide amplified sample for analysis of VNTR, MLVA, and AFLP and can produce defined templates for supplemental analyses, such as by cycle sequencing.

The heating device can be selected from most devices used to heat and cool a substrate, including an external resistive heater such as a nichrome coil; a Peltier device; flowing air, gas, water or other liquids past a device; rapidly moving a microchip from one thermal zone to another; IR heating; heating by alternating current; or other methods well known to function at the macroscale, microscale or smaller dimension, such as continuous flow PCR, where fluid is moved between two spatially separated thermal zones.

Usefully, feedback from a temperature measuring device such as a thermocouple and control software control the temperature regulation to thermally cycle or maintain an isothermal or other profile.

In some embodiments, PCR is modified to accommodate the modular microfluidics of the present invention. Such modifications include, for example, altering the concentration of primers and Mg²⁺; including additives such as BSA, PEG, betaine or other additives to decrease absorption to the walls as the reactions are miniaturized; re-optimizing thermal cycling conditions to minimize offsets in set and actual temperatures, and optimization of key reagent concentrations (DNA, primers, polymerase, DNA to enzyme ratio, and MgCl₂) for microchip reactions, and coating of the channels. Multiplexed PCR reactions, such as those described in U.S. patent application publication nos. 2003/0096291 and 2003/0104459, the disclosures of which are incorporated herein by reference in their entireties, can be implemented to further increase the throughput or information content of reactions.

In some embodiments, stabilized reagents are used. In some of these embodiments, stabilized PCR reagents can be pre-dispensed into the reaction chambers of modular microfluidic devices. As an example, Ready-to-Go.RTM. (RTG) stabilized reagents (GE Healthcare, Piscataway, N.J.) can be used; as commercially available, these kits provide complete PCR reagents including stabilizing carbohydrates, specific primers, premix, and polymerase in a dehydrated form. The reagents are stabilized almost indefinitely at room temperatures by the carbohydrate film until dissolved by adding template DNA. To adapt RTG to microchips, RTG beads can usefully be re-dissolved, the microchip wells coated, and flash-frozen and dried at the bottom of microchip wells, in reaction chambers, in channels or other locations. In other embodiments, real time PCR and/or quantitative PCR (qPCR) can be performed, using standard curves or other methods of calibration to provide quantitative measurement of starting concentrations of template.

PCR reactions can, in some embodiments, be modified to use molecular beacons, TaqMan, or other fluorophores or reporters that perform (fluorescence resonance) energy transfer or quenching reactions or other methods that quantify template starting concentration.

The use of modular microfluidic devices, systems and instruments as disclosed herein provides an additional benefit that the PCR reactions can be performed in multiple microchips and then the endpoint read by moving a microchip onto a fluorescent reader. In an alternative embodiment, the monitoring of fluorescence or other readout can be monitored in a continuous or interval manner by reading directly off of the microchip. Another embodiment performs the PCR amplification with labeling in a modular microfluidic microchip of the present invention and then performs a separation such as capillary electrophoresis, mass spectroscopy, liquid chromatography or other separation methods to separate the products and quantify the amounts.

Rolling Circle Amplification on Modular Microchips

Rolling circle amplification is a technique using a DNA polymerase to replicate DNA in a circular template. Presently phi29 polymerase is used in commercial products. Rolling circle amplification can be used in either linear or exponential amplification mode depending on the primer sets supplied to the DNA polymerase. While rolling circle amplification is a powerful technique that can amplify from single cells and low copy numbers, it produces a very large molecular weight product that has a large physical size compared to microchannels, which can clog microchannels, thereby hindering its adaptation to microfluidic systems.

A disposable sample preparation microchip is loaded with the rolling circle amplification mixture and DNA is added. Alternatively, the complete mixture of DNA, DNA polymerase, and reaction buffers and substrates are premixed and added. The complete reaction is moved into a reaction chamber and incubated at the appropriate temperature. Typically, room temperature is adequate and reaction times are 4 h to 18 h. The long incubation time would provide a rate-limiting block to throughput in a monolithic microfluidic device, where the downstream processes would be kept waiting for the upstream amplification process. In the methods of the present invention, the chip within which amplification is performed would be reversibly segregated from the others of the microfluidic chips during the reaction.

After the amplification reaction is complete, typically a second reaction, such as genotyping or cycle sequencing, is performed. Because the high molecular weight rolling circle amplification product may be hard to move within a channel or chamber, using the modular microfluidic microchip system of the present invention the reaction microchip can dock with a station that injects the next reaction mix and enzyme into the first reaction chamber, the reaction then proceeding in the chamber where the rolling circle reaction has taken place. The second reaction produces lower molecular weight products that can be moved using pressure, electrokinetics, electroosmotics, or other well-known microfluidic motivating means into another channel.

From the channel the sample can either be collected and analyzed off-chip by CAE or other analytical methods or moved to a second microchip using modular microfluidics for analysis on-chip by CAE using a twin-tee injector or other analysis method. In an alternative embodiment, the second reaction is analyzed in place if separations are not required. The microchip containing the rolling circle amplification product can then be discarded or cleaned off-line by methods that digest the large rolling circle amplification product or that fragment it for removal and reuse of the chip if desirable.

Cycle Sequencing Sample Preparation on Microfluidic Devices

For DNA sequencing, cycle sequencing sample preparation can readily be implemented on modular microfluidic microchips. This will produce samples with volumes from 20 μL down to several nanoliters or less and make increased throughput affordable for users.

Cycle sequencing can be performed on modular microfluidic devices, systems and instruments as disclosed herein using dye-terminator or dye-primer chemistries well known in the art. Samples can first be amplified on-chip and analyzed on off-chip in CAE instruments.

For example, in one implementation, dye-terminator sequencing reactions can be used on modular microfluidic devices, systems and instruments as disclosed herein essentially according to the manufacturer's specified protocols using DYEnamic.TM. ET Terminator Sequencing Kits with only slight optimization. Reagents are cycled at 95° C. for 25 s, 50° C. for 10 s, and 60° C. for 2 min for 30 cycles. On-chip thermal cycling can be performed using thermal cycling methods including Peltier heaters, external resistive heaters made from nichrome coils, air-based thermal cyclers, contact fluid thermal cyclers such as “dunking” and switching sources of circulating water or other methods well known in the art.

Temperature control is by software such as the NanoPrep thermal cycler control system with sensing by thermocouples, fluorescent reports such as the Luxtron instrument uses, or other means. Thermocouples can be inserted into a hole in the microchip. Since the thermocouples are not inside the sample chamber, a range of temperatures can empirically determine the offset and optimum. The number of amplification cycles, the temperature profile, and the concentration of different reactants, i.e., primers, polymerase, dNTPs, ddNTPs, etc, are individually optimized.

Buffer additives such as BSA or PVA which can decrease surface effects that remove reactants from the reaction mixture may be added. The surface chemistry of the reaction chambers can be altered using, for example, modified LPA or PEG coatings. For glass, an alternate approach is multipoint covalent attachment of the polymers such as polyethers and oxidized polysaccharides to many surface sites simultaneously, thus extending the lifetime of the surface immobilization since many sites must be hydrolyzed to free the polymer.

The prepared samples can be analyzed “off-chip” in CAE instruments. CAE instruments are capable of detecting sample prepared in 10 nL of sample volume when injection conditions are optimized for small volumes. The samples can provide low volume reactions to feed the CAE throughput. After thermal cycling, for off-chip analysis, samples can dispensed from the microchips into 40 μL of 80% ethanol at room temperature in a microtiter plate by air pressure. For ethanol post-processing, the samples can be centrifuged at 2,800 RCF for 5 s and the alcohol removed by a brief inverted spin for 30 s at 50 RCF. The samples are resuspended in 10 μL of double distilled water. The samples can be subsequently injected into the 96-capillary MegaBACE instrument using a 10 kV, 15 s injection and separated using a120 V/cm field strength. The separation matrix can be 3% linear polyacrylamide (MegaBACE Long Read Matrix, Amersham Biosciences) with a running buffer of Tris-TAPS (30 mM Tris, 100 mM TAPS, 1 mM EDTA, pH 8.0). Four-color electropherograms can be processed using the Sequence Analyzer base-calling software package with the Cimarron 3.1 basecaller (Amersham Biosciences) and the Phred base-calling script. Separations are optimized for injection time, injection voltage, and loading conditions.

In another embodiment, cycle sequencing or genotyping samples can be analyzed by mass spectroscopy. In this method, the length and molecular weight are analyzed to determine the identity of the fragment, particularly for short genotyping reactions. After thermal cycling for on-chip analysis, samples can moved in modular microfluidic devices, systems and instruments as disclosed herein from the sample preparation area to a “twin T” injector for on-chip CAE analysis. With on-chip sample preparation can also be combined with on-chip sample cleanup to improve the capillary electrophoresis.

Sample Cleanup on Modular Microfluidic Devices

A major advantage of sub-microliter sample preparation volumes is that many techniques that are unaffordable at the macroscale can be economically applied to nanoscale samples. Using modular microfluidics, sample cleanup functionality can be provided on a microchip and then the sample moved onto another microchip, or to another part of the same chip, for analysis. The cleanup technology can use beads, particles, monoliths, mini-chromatography, affinity chromatography or other methods well known in the art to purify samples either before sample preparation to remove impurities in the sample; to concentrate or after sample preparation; to remove by-products, reagents, or buffers; to concentrate samples before further processing or analysis; or other applications. The samples can be analyzed on-chip by CAE or another on chip methods, or moved off-chip for CAE, MS, optical imaging, or other analyses.

For example, in one embodiment, for the cleanup of DNA sequencing reactions, the sample cleanup can be implemented using commercially available beads (Agencourt, Dynal, or other vendors) or solid phase beads with custom chemistries on their surface. The chemistries can range from absorption, ion exchange, affinity interactions such as antibodies or biotin, or other chemistries well known in the art. For example, briefly, SPRI beads (Agencourt) are loaded into a channel with a weir to constrain bead flow. The cycle sequencing reactions are then loaded onto the beads and washed with 100% EtOH. After incubation, the beads are washed with 70% EtOH and then the cycle sequencing products eluted with formamide. The concentration of the eluted sample will be kept to a minimal volume of less than 50 nL to keep the samples concentrated enough for injection with a twin tee injector.

In another embodiment, applications of solid phase chemistries on beads, including paramagnetic beads, are used. In another embodiment, affinity purifications can be performed using biotinylated primers, bound antibodies, nucleic acids, or related affinity techniques well known in the art. In this method, primers with biotin on the 5′-end will be loaded onto streptavidin-coated surface and the primers and products bound. The template, salts, and unincorporated nucleotides are washed off before elution of the desired products.

In another embodiment, affinity cleanup methods are used in the modular microfluidic. Acrylamide gels with affinity capture reagents such as antibodies or nucleic acids can be used to capture specific molecules. The affinity capture matrix can collect multiple targets into one sample, as will be needed for MLVA.

In another embodiment, monolith chemistry can be used on modular microfluidic microchips. The monoliths can be made using current technologies, porogens, and modifications to provide a selective separation matrix. The monoliths can be derivatized to have different surface chemistries and separation properties. After cleanup on modular microfluidic microchip, the samples can be transfer using a modular microfluidic interface to an analytical device or the analytical device can be on the microchip.

DNA Fragment Separations

On-chip capillary array electrophoresis with modular microfluidics can be used to separate DNA, RNA, proteins, or other analytes. These include PCR products, cDNA, RNA, single base extension reactions, VNTR, microsatellites, MLVAs and cycle sequencing products. The modular microfluidics provides a mechanism to load samples into a microchip from the top or the end when the sample has been prepared in small volume in a microchip or capillary, or if it has been prepared in larger volumes for analysis on a microchip. The CE separation can also be used as a first dimension in a multi-dimensional analysis or as a later dimension as described below.

Good separations in microchips are possible by systematic optimization of many parameters. A modified version of the Hjerten coating can prevent electroosmosis in the separation channels in glass. Separation matrices can be pumped into the microchip as described in the referenced patents incorporated herein by reference. For example, linear polyacrylamide such as MegaBACE Long Read Matrix performs well as will other formulation including 2% (w/w) high molecular mass (13 MDa) LPA with 0.5% low molecular mass (50 kDa) LPA; DMSO matrix separations; low viscosity POP matrices from Applera; and other matrices such as PVP can be used. For each design, the injection and separation voltage settings for the sample, waste, cathode and anode reservoirs can be tuned. Buffers can be adjusted to increase stacking, minimize injection plug size, provide sufficient ions for separations, and decrease evaporation.

DNA Sequencing on Modular Microfluidic Devices

For DNA sequencing modular microfluidics can prepare and analyze sequencing samples with low consumable costs using automated preparation and analysis. PCR and cycle sequencing microchips can be disposable or reusable devices that are seamlessly integrated with a reusable sequence analysis microchip but could also feed existing conventional CAE instrumentation. By using nanofluidics, a modular microfluidic system can consume fewer reagents and will be less expensive to operate than conventional equipment. The modular approach will be scalable from clinical, research, or high throughput labs; serviceable; and readily extensible as improved microchips are developed.

A DNA sequencing system can be performed by first performing a DNA amplification step, such as the PCR amplification, rolling circle amplification, or other amplification step, as described above and then move the sample to a cleanup step for DNA, such as using biotinylated primers and beads with streptavidin, SPRI, chromatography, or other cleanup methods. The cleaned up sample is then moved either to a reservoir for capillary array electrophoresis off chip or into a twin tee injector for on chip electrophoresis.

Multi-Dimensional Separations

The modular microfluidic devices, systems and instruments as disclosed herein facilitate multi-dimensional analysis. For a multi-dimensional analysis, the first separation dimension might be by free zone capillary electrophoresis and the second dimension by gel capillary electrophoresis. A first dimension of a gel separation on a modular microfluidic microchip could also be used to connect to an electrospray microchip that has a modular connection to receive the sample. The electrospray microchip might have connect to a spray nozzle or have the spray nozzle built into the modular microfluidic microchip. An alternative first dimension is capture onto an affinity material with antibodies, nucleic acids, aptamers, or other affinity materials.

For protein analysis, a two-dimensional separation can be performed by performing the first separation in an isoelectric focusing separation. Pressure is then applied to move the separated sample into an SDS denaturing gel electrophoresis separation. Alternatively, the sample can be released by altering the pH and then electric force used to move the now mobile separated proteins. A modular microchip could also prepare samples by ICAT or other labeling and then perform a first electrophoretic separation before docking with an electrospray MS for downstream analysis.

Similarly, for cell-based analysis, a series of modular microchips could labeled cells and introduce them into a flow cytometer or other readout device for analysis. Other combinations are possible and not meant to be excluded by these examples.

Integration of Sample Preparation, Cleanup, and Analysis

An advantage of the modular microfluidic devices, systems and instruments as disclosed herein is that different steps can be developed individually with off-chip analysis, and then readily integrated into a more complex, multi-chip process using modular microfluidics to transfer samples or fluids. Samples such from cycle sequencing, PCR, cDNA, MLVA, proteins, or other samples can be prepared on a sample preparation microchip and then moved onto a second microchip by pressure, electrical, or other means into a channel or and collected in a reservoir. The samples can be further moved on the second microchip (or a separate location on the same microfluidic chip) into a sample cleanup chamber and prepared as described. After cleanup samples can be eluted into the analysis portion, which can be a “Twin T” CAE system or into a MS or other analytical system. The integration can be by adjusting the workflow to accommodate the linkage of the sample preparation, cleanup, and analysis. This will involve optimizing the concentration of products made in the relevant reaction by changing the number of cycles, incubation times, or starting concentrations; matching the amount of fluid transferred to the concentration of products and elution volumes; and using a “Twin T” injector with sufficient volume to deliver high enough signal coupled with sufficient stacking or initial length to provide adequate resolution. An advantage of the modular microfluidic devices, systems and instruments as disclosed herein is the different steps can be performed on different microchips as appropriate and match microchips with different throughputs.

Detection of Biodefense Agents and Emerging Infectious Diseases

In another application example, rapid detection and analysis of pathogenic organisms is a critical need for biodefense and for the management of emerging infectious diseases. Autonomous systems that can detect pathogens are required in the field while testing laboratories need automated systems that can rapidly detect and fingerprint microbes from human or environmental samples. Systems need to be developed that use advanced technologies including molecular detection, automation, microfluidics, and bioinformatics.

To create a detection monitor, a modular microfluidic device and systems or instrument as disclosed herein can be integrated with an upstream commercially available air sampler such as from Sceptor Industries (Kansas City) or other systems. The output from the air sampler can be lysed on a microchip using sonication, bead beating or other methods on a modular microfluidic microchip. The microchip can then output lysed and concentrated samples into a PCR chamber on a second microchip for on-chip analysis. Alternatively, the sample can be lysed off chip and the sample then feed to the PCR microchip. The PCR sample can be either read by real time PCR in the chamber or if labeled primers are used for multiplex detection, the samples can be separated on a modular microfluidic CAE microchip. Other detection chemistries including immunoassays, isothermal DNA amplification, and preparation of samples for mass spectroscopy are also possible.

Modular microfluidics can provide a platform to develop a biodefense detection system for multiple assays for both field and laboratory settings. In one embodiment, an autonomous air monitor could run a week and perform 336 tests at 30 min intervals or run for a month with 2 hr sampling intervals. For the first screen, the microchips might have up to 12 parallel reactions in a microchip with only one used each interval in low alert levels. The hotels might therefore need to hold at least 40 microchips.

FIG. 2 illustrates one embodiment of apparatus suitable to function as instrument, where the fluid control of a microfluidic device is controlled by reservoir-buffer chamber present on the device as well as remote-valve switch devices controlled by the instrument, to control velocity and directional flow respectively. FIG. 2 shows only one microfluidic device in the instrument, however, the system can easily be adapted by a skilled artisan, and usually will, be multiplexed. Thus, this core technology platform shown in FIG. 2 will be applied to develop autonomous field monitoring equipment with multi-dimensional screening and multiplex screening of multiple samples, as well as a family of laboratory-based instruments.

As an example of how the microfluidic system could work for biodefense monitoring, samples from aerosol might be introduced into the modular microfluidic system. After concentration, the sample, from an air sampler, environmental, or clinical specimen, is moved for testing into microchip. The microchip could be capable of either ELISA or genetic screening, or a combination. PCR amplification with RT-PCR for ten target pathogens might be the primary screen performed on the microchip. If an optical detector senses amplification of a target PCR fragment, the ensuing process could be altered with real-time decision making via control software to trigger a second analysis specific for the putative agent such as fragment size analysis; invoke a MLVA, AFLP, or other assay; and begin a GenomiPhi archival amplification of the sample for subsequent testing. The assay could use microchips already in place or retrieve different microchips. If the secondary screen is positive, a more complete characterization can be initiated with additional assays including short sequencing of pre-defined target regions while a response team arrives to manage the site and download archival material for further testing. Archival samples will consist of unprocessed fractions from the suspect samples and samples that have been processed using the system as disclosed herein, to amplify all the DNA in the sample. Because the systems can be remotely accessed and controlled, the flexible workflow can be used to adjust the sampling rate, focus on specific threats based upon intelligence or other incidents, or change the reporting threshold. The real-time decision making will allow tests to be only conducted when needed, saving assays and reducing costs until pre-defined trigger conditions occur. With web connections, the trigger conditions could be remotely controlled.

VNTR Assay on Modular Microfluidic Devices, Systems and Instruments

VNTR analysis is a method that can be applied to determine the identity and subtype microorganisms. It has uses in epidemiology, biodefense, antibacterial development, medicine and other areas. VNTR can be readily adapted to modular microfluidic microchips. VNTR is based on PCR amplification with primer sets that robustly amplify multiple VNTR targets identified from bioinformatic databases. In modular microchips, the amplification will take place in the same or similar reaction chambers as for PCR reaction example described above. The amplified sets of products can be either removed from the microchip and analyzed on full scale analytical instruments such as capillary array electrophoresis and compared with full volume controls or the fragments analyzed on-chip using capillary electrophoresis on chip. The analysis is by moving the samples into the cross-channel injector and separated on-chip.

The VNTR analysis can be performed on a two chip system or on a three chip modular microfluidic microchip system with on-chip cleanup and analysis by fragment sizing. Without post-processing cleanup of the samples, the dye labeled primers will obscure part of the electropherogram.

MLVA on Modular Microfluidic Devices, Systems and Instruments

The VNTR assays can be extended to Multiple-Locus VNTR Analysis (MLVA). MLVA assays multiple VNTR alleles and provides a fingerprint of an organism. To adapt VNTR to MLVA and modular microfluidics, multiplexed PCR with sets of fluorescently labeled PCR primers is designed to target regions of either the chromosome or plasmids. Bioinformatics and experimental verification well known to one skilled in the art are applied to ensure primer sets do not interact or amplify regions of non-target organisms.

In the modular microfluidic system as disclosed herein, the amplification would take place in the same chamber as the PCR described above, and as disclosed in the Examples and in FIG. 5. The amplified sets of products can be removed from the microchip and analyzed on capillary array electrophoresis (CAE) using denaturing linear polyacrylamide gel and compared with full volume controls. Alternatively MLVA samples can be prepared on one chip, captured on a second cleanup chamber, and analyzed on the third fragment analysis chip as described below.

AFLP on Modular Microfluidic Devices, Systems and Instruments

AFLP is a general fingerprinting technology that appears to be readily adaptable to a modular approach. To adapt ALFP to modular microchips, a restriction digest of lysed cells is first performed on-chip by pumping restriction enzymes and buffer into a chamber with the DNA to be analyzed. Restriction digests have previously been performed and analyzed on microchips. In addition, restriction digests on membranes have been transferred to microchip with the complete digestion and analysis complete within 20 min. The restricted sample can then have fluorescently labeled half-site adapters with two or three nucleotides added and ligated. Fluorescently-labeled PCR primers are added with PCR mix and PCR amplified. This could all occur on one microchip. The sample is the moved to a second microchip and separated by denaturing capillary gel electrophoresis. The fragments are detected by an external laser induced fluorescence detector using charge coupled devices (CCD) or photomultiplier tubes (PMT) or other detectors with laser illumination and appropriate filters. The patterns are analyzed for matches against reference libraries.

Eberwine Amplification

A standard method for amplifying RNA for microarray analysis is using the method commonly called the Eberwine amplification. The Eberwine procedure is a current standard method to linearly amplify RNA for analysis on a DNA microarray to measure global gene expression from total RNA. A transcription amplification using T7 RNA polymerase provides linear amplification which enables the detection of genes with low RNA expression levels.

In this method, after RNA isolation from a sample, first- and second-strand cDNA DNA is created from the RNA with reverse transcriptase using an oligo dT primer linked to a T7 promoter. The resultant DNA is then transcribed with a T7 RNA polymerase to perform a linear amplification that is representative of the original composition of the isolated RNA. The product of the Eberwine amplification is analyzed on DNA microarrays to measure the transcriptional profile of the gene expression of the sample.

The total process can take 18 hours and is a long temporal process even though the number of manipulation steps is few. The long time of the reactions has precluded the adaptation to microchips since, in a monolithic design, the other functions such as for analysis are limited in their throughput to the long incubation times.

Modular microfluidic devices, systems and instruments as disclosed herein can alleviate the problems with adapting the Eberwine process to microchips. The Eberwine process can be performed in reaction chambers on modular microfluidic microchips. The long time can be accommodated by using multiple microchips in parallel. For analysis the prepared Eberwine samples for gene expression analysis are analyzed on full scale gene expression microarrays or in the future the measurements will be in microfabricated chambers on microchips.

Constant Denaturant Gel Electrophoresis

Molecule differentiation methods such as denaturing gradient gel electrophoresis (DGGE), constant denaturant gel electrophoresis (CDGE), and single-strand conformation polymorphism (SSCP) offer the resolving power to identify regions of DNA with mutations compared to reference sequences.

CDCE sample preparation and separations can be performed on modular microchips. The samples can be PCR amplified on a modular microfluidic microchip. Samples from control and PCR amplified test organisms can be mixed together and by using a modular microfluidic interface introduced into a modular microfluidic microchip with multiple separation channels. The samples are separated in non-denaturing gel such as linear polyacrylamide in a range of denaturing conditions; the range can be produced by a heating device or chemical denaturants in the gel. The samples are then electrophoresed and hybrids with control and test strands that differ will be retarded. They can be detected by laser induced fluorescence and other means and then fractions can be collected by moving a receiving modular microfluidic microchip to collect the two homozygous sets of peaks and the heterozygous hybrids. The heterozygous can be further amplified, remixed with controls, and purified. Final analysis can be by DNA sequencing or genotyping methods.

In the future, the modular microfluidic system can be applied to other areas in chemical processing, chemical analysis, biodefense, pharmacogenetics, human medical genetics, biomedical research, animal and plant typing, and human identification.

Although this invention has been described in terms of certain preferred embodiments, other embodiments which will be apparent to those of ordinary skill in the art in view of the disclosure herein are also within the scope of this invention. Accordingly, the scope of the invention is intended to be defined only by reference to the appended claims. All documents cited herein are incorporated herein by reference in their entirety.

In some embodiments of the present invention may be defined in any of the following numbered paragraphs:

1. An integrated microfluidic device for analyzing a sample comprising a planar substrate having;
(i) at least one an input channel adapted to connect to a remote valve, wherein the remote valve can regulate and control the rate and direction of fluid flow on the microfluidic device; and
(ii) at least one high flow chamber and at least one low flow chamber
   2. The integrated microfluidic device of paragraph 1 comprising at least one reservoir to serve as a fluid control buffer to control the flow rate and/or velocity of fluid flow between at least one high flow chamber and at least one low chamber on the planar substrate.
   3. The integrated microfluidic device of paragraph 2, wherein the reservoir receives fluid from a high flow chamber.
   4. The integrated microfluidic device of paragraph 2, wherein the reservoir receives fluid from a low flow chamber.
   5. The integrated microfluidic device of any of paragraphs 1 to 4, wherein the device comprises at least one reservoir which receives fluid from a high flow chamber, and at least one reservoir which receives fluid from a low flow chamber.
   6. The integrated microfluidic device of any of paragraphs 1 to 5, further comprising at least one output channel, where the output channel is adapted to connect to a remote valve, wherein the remote valve can regulate and control the direction of fluid flow on the microfluidic device.
   7. The integrated microfluidic device of any of paragraphs 1 to 6, further comprising at least one output channel fluidly connected to the reservoir, wherein the output channel is adapted to connect to a remote valve, wherein the remote valve can regulate and control the direction of fluid flow on the mircofluidic device.
   8. The integrated microfluidic device of any of paragraphs 1 to 7, further comprising PCR channels in fluid communication with at least one reservoir for thermal cycling of the fluid sample.
   9. The integrated microfluidic device of any of paragraphs 1 to 8, further comprising a sample detection well.
   10. The integrated microfluidic device of any of paragraphs 1 to 5, wherein the high flow chamber is a mixer.
   11. The integrated microfluidic device of any of paragraphs 1 or 5, wherein the low flow chamber is a SPE column.
   12. The integrated microfluidic device of any of paragraphs 1 to 11, wherein the substrate has low auto-fluorescence.
   13. The integrated microfluidic device of paragraph 12, wherein the substrate is Zeonex®.
   14. A system for controlling fluid flow on a microfluidic device of paragraph 1, the system comprising;
(i) a microfluidic device comprising a planar substrate having;
   i. at least one input channel adapted to connect to a remote valve, wherein the remote valve can regulate and control the rate and direction of fluid flow on the microfluidic device;
   ii. at least one output channel adapted to connect to a remote valve, wherein the remote valve can regulate and control the direction of fluid flow on the microfluidic device; and
   iii. at least one reservoir to serve as a fluid control buffer to control the flow rate of fluid flow between chambers on the planar substrate;
(ii) at least one remote valve which is adapted to connect to at least one input channel on the microfluidic device;
(iii) at least one remote valve which is adapted to connect to at least one output channel on the microfluidic device; and
(iv) a control unit connected to each remote valve to control the opening and closing of the remote valves.
   15. The system of paragraph 14, further comprising a thermal controller connected to the control unit, wherein the thermal controller controls the temperature of a thermal interface which interfaces with part of the microfluidic device.
   16. The system of paragraph 14 or 15, further comprising a sample analysis detection system.
   17. The system of paragraph 16, wherein the sample analysis detection system is connected to an optical interface which analyzes a sample present on the microfluidic device.
   18. The system of any of paragraphs 14 to 17, wherein a remote valve control fluid rate using any one of the following selected from the group of; pneumatic dispensers, syringe pumps, or flow restrictors.
   19. The system of any of paragraphs 14 to 18, wherein the microfluidic device is any of the microfluidic device according to paragraphs 1 to 13.
   20. A system for controlling the rate of fluid flow and/or veloctity in at least one channel on a microfluidic device, the system comprising:
   (i) at least one high flow chamber;
   (ii) at least one low flow chamber;
   (iii) at least one reservoir chamber;
   wherein the high flow chamber and the low flow chamber are fluidly connected with the reservoir located therebetween.
   21. The system of paragraph 20, wherein the system further optionally comprising a waste chamber.
   22. The system of paragraph 20, wherein the system further optionally comprising a waste valve.
   23. The system of paragraph 20, wherein the high flow chamber has a flow rate and/or velocity of at least 2 times faster than the rate of the low flow chamber.
   24. The system of paragraph 20, wherein the high flow chamber has a flow rate and/or velocity of at least 3 times faster than the rate of the low flow chamber.
   25. The system of paragraph 20, wherein the high flow chamber has a flow rate and/or velocity of at least 4 times faster than the rate of the low flow chamber.
   26. The system of paragraph 20, wherein the high flow chamber has a flow rate and/or velocity of at least 5 times faster than the rate of the low flow chamber.
   27. The system of paragraph 20, wherein the high flow chamber has a flow rate of at least 200 μl/min, or a velocity of at least 150 mm/seconds.
   28. The system of paragraph 20, wherein the low flow chamber has a flow rate of less than 200 μl/min or a velocity of less than 150 mm/seconds.
   29. The system of paragraph 20, wherein the reservoir chamber has the volume of less than 50 μl.
   30. The system of paragraph 20, wherein the reservoir chamber has the volume of at between 20-50 μl.
   31. The system of paragraph 20, wherein the reservoir chamber has the volume of at least 50 Ul.
   32. The system of paragraph 31, wherein the reservoir chamber has the volume of at least 200 μl.
   33. The system of paragraph 30, wherein the reservoir chamber has the volume of at least 200-1000 μl.
   34. A microfluidic device comprising:
(i) a reservoir with an first input and first output, the first input being in liquid communication through a capillary passageway to the output of a first chamber,
(ii) a first chamber with an input and an output, the output being in liquid communication through a capillary passageway to the first input of the reservoir;
(iii) a second chamber with an input and output, the input being in liquid communication through a capillary passageway to the first output of the reservoir.
   35. The microfluidic device of paragraph 34, further comprising a waste chamber and a waste valve, wherein the inlet of the waste chamber is in liquid communication through a capillary passageway to a second output on the reservoir and wherein the waste valve controls flow of fluid from the reservoir second output into the inlet of the waste chamber, wherein when the waste valve is in the open position, fluid flows from the reservoir second output into the inlet of the waste chamber.
   36. The microfluidic device of paragraphs 34 and 35, further comprising a second input on the reservoir chamber, wherein the second inlet of the reservoir chamber receives a liquid to displace the liquid in the reservoir via the first output into the second chamber or via the second output into the waste chamber.
   37. The microfluidic device of any of paragraphs 34 to 36, wherein the first chamber is a high flow chamber, and the second chamber is a low flow chamber.
   38. The microfluidic device of any of paragraphs 34 to 36 wherein the first chamber is a low flow chamber, and the second chamber is a high flow chamber.
   39. The microfluidic device of any of the paragraphs 34 to 38, wherein the reservoir has the volume of less than 50 μl.
   40. The microfluidic device of any of the paragraphs 34 to 39, wherein the reservoir chamber has the volume of between 20-50 μl.
   41. The microfluidic device of any of the paragraphs 34 to 40, wherein the reservoir chamber has the volume of at least 50 μl.
   42. The microfluidic device of any of the paragraphs 34 to 41, wherein the reservoir chamber has the volume of at least 200 μl.
   43. The microfluidic device of any of the paragraphs 34 to 32, wherein the reservoir chamber has the volume of at between 200 μl-100 μl or greater than 1000 μl.
   44. The microfluidic device of paragraph 37 or 38, wherein the high flow chamber has a flow rate and/or velocity of at least 2 times faster than the rate of the low flow chamber.
   45. The microfluidic device of paragraph 37 or 38, wherein the high flow chamber has a flow rate and/or velocity of at least 3 times faster than the rate of the low flow chamber.
   46. The microfluidic device of paragraph 37 or 38, wherein the high flow chamber has a flow rate and/or velocity of at least 4 times faster than the rate of the low flow chamber.
   47. The microfluidic device of paragraph 37 or 38, wherein the high flow chamber has a flow rate and/or velocity of at least 5 times faster than the rate of the low flow chamber.
   48. The microfluidic device of paragraph 37 or 38, wherein the high flow chamber has a flow rate of at least 200□/min or a velocity of at least 150 mm/sec.
   49. The microfluidic device of paragraph 37 or 38, wherein the low flow chamber has a flow rate of less than 200 □l/min or a velocity of less then 150 mm/sec.
   50. The use of the microfluidic device of any of paragraphs 1 to 13 or 34 to 49 to increase or decrease the velocity and/or flow rate in a microfluidic device.
   51. The use of the microfluidic device of any of paragraphs 1 to 13, or 34 to 49, to decrease the velocity and/or flow rate in a microfluidic device.
   52. The use of the microfluidic device of any of paragraphs 1 to 13 or 34 to 49 to increase the velocity and/or flow rate in a microfluidic device.
   53. A method of decreasing the rate of fluid flow on a microfluidic device, comprising:
(i) opening an waste valve which controls output flow of a reservoir chamber;
(ii) dispensing at least one liquid from a high flow rate chamber into the input of the reservoir, wherein the liquid from the high flow rate chamber displaces the fluid in the reservoir into a waste chamber;
(iii) closing the waste valve which controls output flow of a reservoir chamber;
(iv) dispensing the at least one liquid from the reservoir into a low flow rate chamber.
   54. An remote-valve microfluidic device for controlling the flow of fluid in at least one channel on a microfluidic device; comprising;
   (i) a fluid-impermeable substrate, the substrate having at least one channel, the channel having an inlet on one external surface and an outlet at a different external surface,
   (ii) a valve, the valve configured to be in an open position to allow fluid to pass through the channel from the inlet to the outlet or configured to be in a closed position to completely interrupt the flow of fluid from the inlet to the outlet,
   wherein the external surface of the substrate with the inlet is fashioned for a reversible, fluidly sealed, engagement to an external surface of a second microfluidic device, creating an interface therebetween, and
   wherein the channel of the substrate is capable of fluidly communicating with a channel on a second microfluidic device across the interface.
   55. The remote-valve microfluidic device of paragraph 54, wherein the channel of second microfluidic has an inlet and an outlet, where the outlet is on the external surface of the second microfluidic device which forms the interface.
   56. The remote-valve microfluidic device of paragraph 54, wherein the channel of second microfluidic has an inlet, at least one junction and at least two arm channels coming off the junction, each arm channel having an outlet on the same or different external surfaces of the second microchip.
   57. The remote-valve microfluidic device of paragraph 54, optionally comprising a fluid impermeable material at the interface between the external surface at the second microfluidic device and the external surface of the substrate.

58. The remote-valve microfluidic device of paragraph 54, wherein the second microfluidic device is according to any of paragraphs 1 to 13 or 34 to 49.

59. An system for remotely controlling the fluid flow in at least one channel on a microfluidic device, the system comprising:
(i) at least one microfluidic device having at least one channel, said channel having at least one input and at least one output, wherein at least one input or at least one output are on the external surfaces of the microfluidic device;
(ii) a plurality of remote-valve microfluidic devices of paragraph 1, each containing a valve and at least one channel bias towards and reversibly sealed to, at least one microfluidic chip, creating in interface therebetween; wherein the channel of each remote-valve microfluidic device is capable of fluidly communicating with an input or output of a channel on the microfluidic device across the interface;
(iii) a means for opening and closing the valve in at least one remote-valve microfluidic device, wherein an open valve position allows fluid to flow across the interface between the channel of the remove valve microfluidic device and the channel of the microfluidic device, whereas a closed valve position prevents the flow across the interface between the channel of the remove valve microfluidic device and the channel of the microfluidic device.
   60. The system of paragraph 59, wherein the channel of the microfluidic device has an inlet, at least one junction and at least two arm channels coming off the junction, each arm channel having an outlet on the same or different external surfaces of the second microchip.
   61. The system of paragraph 59, optionally comprising a fluid impermeable material interface between the microfluidic device and the remote-valve microfluidic device.
   62. The system of paragraph 59, wherein the microfluidic device is according to paragraph 15 to 30.
   63. An apparatus for reversibly integrating a plurality of modular remote-valve devices, each containing at least one capillary channel and one valve, into a fluidically communicating system, the apparatus comprising: means for reversibly biasing each of a plurality of remote-valve devices towards at least one microfluidic device with sufficient bias to create a reversible fluidically sealed interface therebetween; means for actuating a valve of the plurality of remote valve devices, wherein the valve in of the remote valve device directs the flow of liquid of the microfluidic device to which the remote-valve device is reversibly and fluidly sealed to.
   64. The apparatus of paragraph 63, wherein the apparatus is capable of reversibly biasing a first microfluidic device into fluidically sealed engagement with a remote-valve microfluidic device.
   65. The apparatus of paragraph 63, wherein the apparatus reversibly fixes one of the plurality of microchips into place.
   66. The apparatus of paragraph 63, wherein the apparatus further comprises fluid motivating means.
   67. The apparatus of paragraph 63, wherein the apparatus further comprises detection means.
   68. The apparatus of paragraph 67, wherein the detection means are optical detection means.
   69. The apparatus of paragraph 68, wherein the detection means is a sample analysis detection system which is connected to a computer, and wherein the optical detection means can detect fluorescence of a sample present in the microfluidic device.
   70. An apparatus, comprising;
(i) at least one microfluidic device having at least one channel, said channel having at least one input and at least one output, wherein at least one input or at least one output are on the external surfaces of the microfluidic device;
(ii) a plurality of remote-valve microfluidic devices of paragraph 1, each containing a valve and at least one channel bias towards and reversibly sealed to, at least one microfluidic chip, creating in interface therebetween; wherein the channel of each remote-valve microfluidic device is capable of fluidly communicating with an input or output of a channel on the microfluidic device across the interface;
(iii) a means for opening and closing the valve in at least one remote-valve microfluidic device, wherein an open valve position allows fluid to flow across the interface between the channel of the remove valve microfluidic device and the channel of the microfluidic device, whereas a closed valve position prevents the flow across the interface between the channel of the remove valve microfluidic device and the channel of the microfluidic device.
   71. The apparatus of paragraph 70, wherein the microfluidic device is a valveless microfluidic device.
   72. The apparatus of paragraph 70, wherein the microfluidic device is a microfluidic device of any of paragraphs 1 to 13 or 34 to 49.

### EXAMPLES

The examples presented herein relate to a method of controlling fluid flow on a microfluidic device, in particular one aspect of the present invention relates to the directional control of fluid along channels on a valueless microfluidic devices using remote valve switching microfluidic device, and another aspect of the present invention relates to the control of flow rate on a microfluidic device, in particular changing the flow rate on a microfluidic device using a reservoir-buffered mixers. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Herein, the inventors have demonstrated the design and function of a low-cost to manufacture planar chip to identify bacterial pathogens in liquid clinical samples (FIGS. 2 and 5). In some embodiments, the chip includes a channel filled with a porous polymer monolith (PPM) embedded with silica particles for the lysis of bacteria and the isolation of the released nucleic acids¹¹. The PPM channel acts as an on-chip solid phase extraction (SPE) column and has been shown to isolate nucleic acids from bacteria¹ and viruses¹² in various human physiological samples including urine¹, blood¹³, and stool¹⁴. In some embodiments, the chip includes zig-zag mixers to mix reagents, fluid reservoirs to allow different fluid velocities through various regions of the chip, a PCR chamber, and an optical detection well for an end-point fluorescence measurement. The inventors in one embodiment made the chip from Zeonex® plastic as it has a high glass transition temperature to sustain the temperature range required by PCR, has a good UV transmissibility to allow in situ UV-curing of the PPM, and low auto-fluorescence to allow on-chip fluorescence-based detection of amplicons.

With a completely passive chip, an instrument is required to conduct the fluidic, temperature, and optical control. Although the point-of-care market will require a small inexpensive instrument, the inventors herein demonstrate, using an initial prototype instrument the significant flexibility in the design to enable it to be adapted to any microfluidic device, as well as exploration of the experimental protocol.

In one embodiment, the inventors included multiple syringe pumps and air pressure driven mechanisms and valves for fluid control, a ceramic heater with an air cooler for temperature control and a spectrophotometer for fluorescence measurements (see FIG. 2A). This design enabled the inventors to study various fluidic, temperature, and optical experimental methodologies to optimize the system, and can be adapted for specific microfluidic analysis and detection methods.

To enable fluid directional control without on-chip valves, the inventors developed and demonstrate use of a remote valve switching concept which utilizes the inherent incompressibility of liquids to control the fluid directions with valves located on the instrument. In other embodiments, the instrument can be made significantly smaller and less expensive than this original prototype.

To demonstrate the full functionality of the chip and instrument, the inventors present fluorescence data taken on-chip and the corresponding off-chip gel electrophoresis of the resultant amplicon for input samples of Bacillus subtilis DNA or bacteria.

### Material and Methods:

Microfluidic Chip prototype manufacture and preparation: The chip features were machined with a computer numerical controlled (CNC) milling machine in ZEONEX® 690R, obtained as molded plaques from Zeon Chemicals (Louisville, Ky.). The chip was designed to be manufactured via injection molding, however all work presented in this paper was conducted with milled chips to allow for rapid prototyping of various designs. To seal the channels, a cover slip was cut from 0.010 in. extruded Zeonex® 690R film (Plitek, Inc., Des Plaines, Ill.) and bonded to the chip with a solvent-assisted thermal bonding methodology¹⁵. To accomplish the bonding, the milled chip and cover slip were soaked for 1.5 hours in a sealed chamber containing toluene vapor. After vapor treatment, the chip and cover slip were thermally bonded in a Carver 4386 Hot Press at 271° F. for 2 min with the cylinder pressure set at 7757 (1125 psi). The piston has a 4.75 cm ID and the chip area is 46.5 cm².

Once the microfluidic chip was sealed, the porous polymer monolith (PPM) with embedded silica particles was polymerized via UV-curing in the “SPE column” 50 (see FIG. 5A). The PPM was manufactured in a modified method as previously published¹¹ and is briefly described here. The PPM is made in a two-step process that first prepares the channel side walls with a thin layer of grafted polymer, followed by filling the channel with a UV-cured monolith. Butyl methacrylate (99%, BuMA), ethylene dimethacrylate (98%, EDMA), ethylene diacrylate (90%, EDA), methyl methacrylate (99%, MMA), 1-dodecanol (98%), cyclohexanol (99%), benzophenone (99%), and 2,2-dimethoxy-2-phenylacetophenone (99%, DMPAP) were purchased from Sigma-Aldrich (St. Louis, Mo.). The grafting solution was prepared with 3.88 mL MMA and 120 mL melted Benzophenone. The SPE monomer mixture was prepared with 960 μL BUMA, 640 μL EDMA, 1680 μL 1-Dodecanol, 720 μL Cyclohexanol, and 16 μL melted DMPAP. 4.0 mL of 0.7 μm silica microsphere (Polysciences, Inc., Warrington, Pa.) were centrifuged at 2500 g for 10 min, the supernatant decanted, and then dried at 120° C. for 1 hr to a hard pill. The pill was broken into a power and mixed with the SPE monomer mixture, and sonicated in a Branson sonicator until silica dispersed (2-3 min). The ends of the channel were blocked with a water soluble hydroxyethylcellulose jelly (K-Y Jelly, Personal Products Company, Skillman, N.J.). Grafting solution was pipetted into channel through access holes and exposed for 10 min in a CL-1000 crosslinker (UVP, Inc., Upland, Calif.). After light exposure, the excess solution was vacuumed out. Then, the SPE monomer mixture was pipetted into the channel through the access holes and exposed to UV radiation for 15 min per side of the chip to polymerize the material. The access holes were then sealed shut with J-B Weld epoxy (J-B Weld, Sulpur Springs, Tex.). The resultant SPE column was rinsed with methanol for 15 min at 50 psi to remove any residual porogenic solvents or un-polymerized monomers. To remove traces of toluene, the chip was placed in the instrument (FIG. 2) and the PCR channel was rinsed with water for 1 hour with the on-chip thermal cycler set to 95° C. Finally to sterilize the chip for bacterial detection assays, it was rinsed sequentially with ˜1 mL each of 10% bleach solution, sterilized water, and methanol, before being dried with vacuum.

Bacillus subtilis culture and off-chip DNA isolation: A non-chain forming B. subtilis strain 168 (a gift from Shigeki Moriya, Institute for the Biotechnology of Infectious Diseases, Sydney, Australia) was cultured under aerobic conditions at 37 deg C. on an orbital shaker at ˜200 rpm, using LB broth (Lennox) media (Fisher Scientific, Pittsburgh, Pa.). A standard curve for B. subtilis growth was prepared by measuring the optical density (OD) of cells at 600 nm at different time points and by plating serially diluted cells onto LB-Agar (1.5%). Colonies were counted after 12 hours of cell growth and correlated with the OD of the cells at 600 nm. All subsequent cell counts were calculated by measuring the OD of cells at 600 nm and calibrating against the standard curve.

Genomic DNA was isolated from 50 mL of exponentially growing B. subtilis cells using the Qiagen DNeasy Blood and Tissue kit (Valencia, Calif.) as per manufacturer's instructions. The quality of the genomic DNA was tested by measuring optical density at 260 nm and by gel-electrophoresis on 6% polyacrylamide gel (Invitrogen Corporation, Carlsbad, Calif.) using Tris-borate-EDTA buffer as per manufacturer's protocol. Gels were visualized by Sybr-green based DNA-staining kit (Invitrogen Corporation).

Bacillus subtilis PCR: The inventors chose the B. subtilis gene ftsA, coding for an actin like cytokinetic protein responsible for cell division, as our marker for B. subtilis identification. The primers (GATCACCGGTTCAAAAACAATCTTACA (SEQ ID NO: 1), AGCGGCTGAAGGCAAATATCA; (SEQ ID NO: 2)) were targeted against the middle of the ftsA sequence which results in a PCR product that is 90 basepairs long, and were designed using the custom primer design tool for Taqman assays (Applied Biosystems, Foster City, Calif.). The Taqman assay primers and the Taqman assay master mix were purchased from Applied Biosystems and the off-chip PCR reactions were run on an ABI7300 (Applied Biosystems) real-time PCR instrument as per Applied Biosystems' recommended protocol. The thermal profile for off-chip PCR was an initial hold of 55° C. for 3 minutes, a 95° C. hold for 5 min, followed by forty cycles of 95° C. for 15 s and 60° C. for 1 minute. The on-chip PCR thermal profile consisted of an initial hold of 93° C. for 10 min, followed by 40 cycles of 93° C. for 30 s and 58° C. for 30 s. The detection of off-chip PCR products was achieved by real-time PCR (FAM dye) using Taqman-specific detection protocols in an ABI7300 instrument and through electrophoresis on 6% polyacrylamide gels (Invitrogen corp, USA) using Tris-borate-EDTA buffer as per the manufacturer's protocol. Gels were visualized by Sybr-green based DNA-staining kit (Invitrogen Corporation, USA).

On-chip reagents: Four solutions were used for the on-chip protocol: 1) the chaotropic buffer, also designated as the sample propulsion buffer, 2) the SPE column wash buffer, 3) the elution buffer, also designated as the elution propulsion buffer, and 4) the PCR mix. The chaotropic buffer consisted of a 50:50 mixture of 3M guanidium thiocyanate (GuSCN, Sigma-Aldrich) and isopropyl alcohol (Sigma-Aldrich). The two part reagent was used simultaneously to aid in the lysis of the bacteria by providing a chaotropic agent and to drive any released nucleic acids to bind to the silica particles embedded in the PPM with the organic solvent. The SPE column wash buffer was 70% ethanol and was used to rinse the SPE column of any contaminants (proteins, lipids, residual chaotropes, etc.). The nucleic acids were then eluted from the SPE column with an elution buffer (10 mM Tris-Cl, 1 mM EDTA, Fisher Scientific). Next, the eluate was mixed with the appropriate primers/probes, blocking agents, enzymes, and buffers (PCR mix). The PCR mix consisted of one part 20× primer-probe custom ordered from Applied Biosystems (see Bacillus subtilis PCR section above), 0.6 part 3.5% bovine serum albumin (Sigma Aldrich), and 10 parts Taqman assay master mix (Applied Biosystems). The instrument automatically delivered appropriate volumes of these reagents according to the protocol defined by the user.

Optics and signal processing: Following thermal cycling, the optical system measured the resultant fluorescent signal of the thermally cycled mixture in the optical detection well. The excitation optics consisted of an Ocean Optics LS-450 Blue LED light source (Dunedin, Fla.), filtered through a Thorlabs FES 0500 (Newton, N.J.), and focused with an Edmund Optics NT45-081 (Barrington, N.J.). A mirror (Thorlabs ME05-P01) was positioned above the detection well to reflect any emitted fluorescence back towards the detector (Ocean Optics USB4000 Spectrometer). Spectral data were gathered from the spectrophotometer and nulled against a dark spectrum to remove noise and detector artifacts. To compensate for drift in the LED intensity, the spectra were normalized against the height of the excitation peak. In practice, an initial baseline spectrum was taken while the PCR reaction products were still in the PCR channel; which included signals from stray light, excitation light, autofluorescence of the chip, and background signal from the dye. Then, the reaction products were pushed into the detection well, and the assay spectrum was taken. The reported assay signal consisted of the assay spectrum minus the baseline spectrum.

### Example 1

System Design. The lab-on-a-chip system was designed to accomplish the following steps:
1. input 50-400 μL of liquid physiological sample containing a pathogen,
2. mix the sample with a chaotropic buffer to aid in the release of nucleic acids,
3. flow the mixture over the SPE column to further lyse any remaining intact pathogens and bind the nucleic acids on the SPE column,
4. wash the SPE column of proteins and chaotropic agents,
5. dry the SPE column to remove any residual organic solvents,
6. elute the nucleic acids,
7. mix the eluate with PCR master mix,
8. thermally cycle the PCR mixture to amplify the target gene,
9. and detect the resultant amplicon via an end-point fluorescence measurement.

The system includes two major components: a disposable single-use plastic chip and an instrument which houses all active components to truly make the disposable low cost. To further minimize the cost of the chip, the microfluidic channels and chambers are in a planar geometry amenable to injection molding, and so the disposable can be assembled with a minimum number of steps. The final chip is composed of a chip with microfluidic features, bonded to a plastic cover slip.

To achieve the goal of removing all active components from the disposable chip, three fluid control design elements were implemented. First, to eliminate the need for on-chip valves, the inventors designed the fluidic system to use a remote valve switching concept. The fluidic pumps and valves needed to drive the various fluids on-chip are located in the instrument (see FIG. 2 for instrument and functional layout). The chip interfaces with the instrument by mating to an interface block which is sealed with an fluid impermeable material, for example, rubber o-rings, or other equivalent sealing mechanism at each of the fluid inputs and outputs. The remote valve switching takes advantage of the inherent incompressibility of liquids such that once all the fluid lines are full, the fluids in the chip can be driven by remote pumps and switched by remote valves. For instance, suppose that a channel branches to two outputs, A and B. Both outputs connect to tubes which lead into the instrument, where they pass through valves A and B. To switch the flow out through A, the instrument opens valve A and closes valve B. As long as there is no compressibility in the entire path between the branching point and valve B, then no fluid can flow into channel B, and thus it will all flow through output A. This effectively works like a valve located at the branch point, but is far cheaper and easier to implement. In order for this design to work, the channel needs to be stiff, in order to avoid a small amount of liquid being displaced in the ‘closed’ channel. The inventors successfully demonstrated in making the channel of sufficient stiffness so that only a very small volume or negligible was displaced.

To utilize the remote valve switching methodology, the chip and fluid lines must be pre-filled with liquid. Therefore, the second design element is that a priming step is required at the beginning of each experiment to ensure the fluid channels are filled. Third, to eliminate the need for active mixers on-chip, the inventors implemented simple zig-zag mixers. These mixers are quite simple in design, but require a relatively high fluid flow rate to achieve thorough mixing¹⁶. In contrast, the fluid flow rate of the sample through the SPE column must be relatively slow to allow time for nucleic acid binding to the silica particles and to maintain the structural integrity of the PPM matrix. To reconcile these two competing needs, the inventors added to the chip design two fluid reservoirs to enable multiple fluid velocities on the same chip using the remote fluid control concept.

To explore the possibility of low-cost instrumentation, the inventors implemented two different fluid propulsion methodologies; syringe pumps and pneumatic dispensers. The syringe pumps are very accurate and deliver well quantified volumes, but are relatively expensive. The pneumatic dispensers use compressed air to drive the fluids through a flow restrictor. To deliver the fluid volume accurately, tight control of the applied pressure and valve timing must be maintained. Pneumatic dispensers can be implemented inexpensively and thus represent the preferred implementation for low-cost instrumentation if adequate fluid control can be achieved. The first prototype instrument was intended to allow for flexibility and thus has extra pumps for different chip designs. For the chip described here, three syringe pumps are used to control the delivery of the chaotropic buffer (guanidium thiocyanate (GuSCN) and isopropanol), sample propulsion buffer, and elution buffer. Pneumatic dispensers control the delivery of the compressed air, 70% Ethanol, and PCR master mix. The various pumps and valves are controlled by a host personal computer (PC) (see FIG. 2B) via custom software written in Think and Do (Phoenix Contact, Middletown, Pa.). After implementation, the inventors found that the pneumatic dispensers performed well and were almost as reliable as the much more costly syringe pumps. For the system described here, the pneumatic dispenser approach is in fact sufficient and future designs can make use of this low cost fluid control method.

The fluidic protocol through the chip (FIG. 5A) is as follows: First, the fluid lines and the microfluidic channels are filled with their respective reagents to prime the system and remove any bubbles. Then, up to 400 μL of sample is loaded into the sample reservoir (“sample in”) 10. The sample is propelled with sample propulsion buffer and mixed with a chaotropic buffer (Inlet labeled “GuSCN” 20) in “Mixer 1” 30 at a fluid flow rate of 8 μL/s. The mixture is collected in “Reservoir 1” 40 to allow for a fluid flow rate change. The mixture is then loaded at a flow rate of 0.18 μL/s onto the “SPE column” 50 to lyse any residual intact bacteria and bind the released nucleic acids. The column flowthrough is directed to “Waste 1” 60. The SPE column is then washed with 70% ethanol from the port so-named and the flowthrough exits the chip out “Waste 1” 60. The SPE column is dried with compressed air from the “Air” 70 inlet and out through “Waste 1” 60 to remove any residual organic solvents, which could inhibit the subsequent PCR. The nucleic acids are then eluted from the column with elution buffer from the “Elution Buffer” 80 port and joined with a stream of PCR master mix from inlet “PCR mix” 90. The parallel streams are collected in “Reservoir 2” 100 to allow for a fluid flow rate change to the high mix speed through “Mixer 2” 110. The mixture is then pushed by “Eluate Propulsion Buffer” 120 into the “PCR channel” 130 at 8 μL/s. After thermal cycling, the mixture is pushed by “Eluate Propulsion Buffer” into the “Detection Well” for optical read-out. To ensure the proper fluid control, the inventors visualized the fluid movement by staining the input fluids with food coloring.

The thermal heating of the PCR channel was achieved by placing a ceramic heater in direct contact with the chip. The cooling was achieved by blowing room temperature air on the cooling fin located on the heater's underside. The chip was insulated on the opposite side of the chip from the heater to minimize heat loss and maintain close to uniform temperature throughout the PCR mixture. To measure the temperature response of the thermal control system, the inventors embedded a thermocouple into a “dummy” chip. The ramp rate was adjusted to minimize the cycle time and the temperature overshoot. The inventors achieved a heating ramp rate of ˜10° C./s and a cooling ramp rate of ˜5° C./s, which compares favorably to commercial instruments (e.g. Applied Biosystems 96-well GENEAMP® 9700 with a ramp rate of 5° C./s) (FIG. 11A).

The detection optics consist of an excitation source and a detector, both located underneath the chip and oriented 90 degrees from one another. A broadband mirror is also positioned above the chip, to reflect more of the emission to the detector. The excitation source is an LED with a center wavelength of 470 nm, which is filtered through a short-pass filter with a cutoff wavelength of 500 nm. To enable the greatest flexibility in fluorescence detection, the inventors chose to integrate a spectrophotometer as our detector. The spectrophotometer measures light intensity for a broad range of wavelengths. The software can either collect a whole spectrum of optical data or a single wavelength such as 525 nm (the peak emission of FAM, the dye used here). To assess the optical sensitivity of the system, the inventors measured the fluorescence from serially diluted, off-chip PCR-amplified DNA from B. subtilis using a Taqman assay (FIG. 11B). In these experiments, each copy of the amplicon releases a single reporter FAM dye molecule. The inventors estimated the concentration of the 90 basepair amplicon with a UV absorbance measurement (ratio of optical density at 260 nm and 280 nm) to be approximately 8.1 μg/mL after 40 cycles of amplification. From the calibration curve, the inventors estimate that the optical system can detect less than 1 ng/μL of amplicon using a Taqman assay (FIG. 11B). For the chip's 50 μL PCR reaction channel volume, this concentration of amplicons is achievable from a single starting target molecule in ˜38 cycles. As such, the inventors typically ran the on-chip PCR for 40 cycles.

The different subsystems were tested to confirm on-chip functionality and compared to off-chip standard methods. For example, PCR reagents were placed in the PCR channel and then thermal cycling and optical detection were conducted on-chip. After on-chip optical detection, the contents of the detection well were collected and the presence of the amplicon confirmed by gel electrophoresis. The function of the SPE column was confirmed by introducing a sample of purified B. subtilis DNA in 1×TE buffer in the sample reservoir and running the on-chip fluidics protocol up through the nucleic acid elution step. Then the eluate was collected from the chip and the presence, quality and quantity of the DNA was assessed by off-chip real time PCR (FIG. 12B). After ensuring the subsystems of the chip were functioning properly, the inventors tested the function of the complete lab-on-a-chip by introducing 20 ng of purified B. subtilis genomic DNA into the sample reservoir. The automated protocol was run. After end-point optical detection, the presence of the amplicon was confirmed by gel electrophoresis (FIG. 12B). For each chip, a full process negative control (water in the sample reservoir) was conducted followed by the DNA sample. Representative optical signals and gel electrophoresis for the negative controls and DNA samples are shown in FIG. 12A. The endpoint fluorescence signals for 20 ng of input DNA are on average 3500 fluorescent units above the on-chip negative controls, clearly demonstrating detection of the input DNA. The variability in the output signal is due to variations in the performance of the SPE channel, loss to the sidewalls of the chip, and the inherent variability of an endpoint fluorescence measurement for PCR.

### Example 2

Bacterial detection: To demonstrate the utility of the fully functional chip for detection of bacteria, the inventors conducted a series of experiments with different numbers of input B. subtilis cells (1.25×10⁶, 6.25×10⁶, or 12.5×10⁶ cells). For each chip, the inventors first ran a full-process negative control (water input) followed by the full process with the B. subtilis cells. The inventors present the optical detection data as the difference between the signal with B. subtilis and the same chip's negative control in FIG. 13A. After the on-chip detection was completed, the inventors collected the contents of the detection well and analyzed the sample by gel electrophoresis to confirm the presence of the target amplicon (FIG. 13B).

All three concentrations of B. subtilis were detected above the negative signal demonstrating that the system can successfully lyse the bacteria and isolate the nucleic acids, PCR amplify the target, and detect the presence of the amplicon via fluorescence optical measurements. Although the endpoint fluorescence data varies with the input concentration, the methodology is not strictly quantitative as the fluorescence measurement is not conducted concurrently with the thermal cycling. As the B. subtilis genome is 4.2 Mbps, the 20 ng of genomic content introduced for the experiments presented in FIG. 12 corresponds to approximately 4.2×10⁶ cells. Comparing the endpoint fluorescence signals from the samples containing purified DNA and those with B. subtilis cells implies that the bacterial lysis is not completely efficient. In some embodiments, cell lysis can be enhanced by integrating a cell lysis high-flow component as disclosed in WO2009/002580, upstream of the mixer to improve cell lysis. Nonetheless, the system presented here demonstrates the end-to-end detection of bacteria in a model system.

### Example 3

The main contributions described in this article are the design, implementation and demonstration of an end-to-end lab-on-a-chip system for the detection of bacteria that is truly low cost to manufacture. The field of microfluidic total analysis systems promises low-cost systems by miniaturizing and automating traditionally labor and reagent intensive processes. However, many of the innovations are complicated and expensive to manufacture and/or made of materials that are not robust for commercial application. To minimize the complexity and cost of the disposable component, the inventors designed a completely passive chip in a planar format that can be injection molded. The inventors have developed the injection molding process for this chip and will present the manufacturing methodology elsewhere. To achieve the full integration and automation of the laboratory steps required (sample and reagent introduction, mixing, nucleic acid isolation, PCR and optical detection), the inventors employed a remote valve switching concept in combination with fluid reservoirs to allow fluid flow rate changes. Additionally, the inventors incorporated a proven microchannel compatible SPE column¹¹. These innovations allow the chip to be manufactured in a low cost methodology with minimum assembly steps. The instrument can also be readily adapted to a lower cost solution and smaller footprint by implementing pneumatic dispensing for the whole system and replacing the spectrophotometer with filtered photodiode optics and a narrow bandwidth LED/PMT.

The inventors chose to demonstrate the full functionality of the chip with B. subtilis as a model system because it is a gram positive bacterium with a thick peptidoglycan cell wall. In general, gram positive bacteria are more difficult to lyse due to their cell wall than gram negative bacteria or viruses. The chip protocol was designed to include both chemical lysis by addition of chaotropic agents and mechanical lysis by shearing the bacteria at relatively high pressures against the porous polymer monolith in the SPE column. The methodology is therefore expected to be easily extendable to gram negative bacterial targets¹ or viral targets¹². Moreover, as the PPM technology has been shown to be effective at isolating nucleic acids from physiological samples^{1, 13, 14}, the inventors believe the system is also readily extendable to detect pathogens from clinical samples and future work will be directed towards such a demonstration.

Hence, this work describes a significant step towards the implementation of microTAS to replace traditional clinical nucleic acid analysis, which has applicability in modern clinical labs as well as in more challenging environments such as at the patient point-of-care or in resource limited settings.

### REFERENCES

All references cited in the specification and the Examples are incorporated herein in their entirety by reference.
1 M. D. Kulinski, M. Mahalanabis, S. Gillers, J. Y. Zhang, S. Singh and C. M. Klapperich, Sample preparation module for bacterial lysis and isolation of DNA from human urine, Biomed. Microdevices, 2009.
2 J. B. Henry, ed., Clinical Diagnosis and Management by Laboratory Methods, Saunders, Philadelphia, 2001.
3 P. E. Andreotti, G. V. Ludwig, A. H. Peruski, J. J. Tuite, S. S. Morse and L. F. Peruski, Jr., Immunoassay of infectious agents, BioTechniques, 2003, 35, 850-859.
4 D. Ivnitski, D. J. O'Neil, A. Gattuso, R. Schlicht, M. Calidonna and R. Fisher, Nucleic acid approaches for detection and identification of biological warfare and infectious disease agents, BioTechniques, 2003, 35, 862-869.
5 G. W. Procop, Molecular diagnostics for the detection and characterization of microbial pathogens, Clin. Infect. Dis., 2007, 45 Suppl 2, 599-5111.
6 C. D. Chin, V. Linder and S. K. Sia, Lab-on-a-chip devices for global health: past studies and future opportunities, Lab Chip, 2007, 7, 41-57.
7 P. A. Auroux, Y. Koc, A. deMello, A. Manz and P. J. Day, Miniaturised nucleic acid analysis, Lab Chip, 2004, 4, 534-546.
8 L. Chen, A. Manz and P. J. Day, Total nucleic acid analysis integrated on microfluidic devices, Lab Chip, 2007, 7, 1413-1423.
9 C. J. Easley, J. M. Karlinsey, J. M. Bienvenue, L. A. Legendre, M. G. Roper, S. H. Feldman, M. A. Hughes, E. L. Hewlett, T. J. Merkel, J. P. Ferrance and J. P. Landers, A fully integrated microfluidic genetic analysis system with sample-in-answer-out capability, Proc. Natl. Acad. Sci. U.S.A., 2006, 103, 19272-19277.
10 R. H. Liu, J. Yang, R. Lenigk, J. Bonanno and P. Grodzinski, Self-contained, fully integrated biochip for sample preparation, polymerase chain reaction amplification, and DNA microarray detection, Anal. Chem., 2004, 76, 1824-1831.
11 A. Bhattacharyya and C. M. Klapperich, Thermoplastic microfluidic device for on-chip purification of nucleic acids for disposable diagnostics, Anal. Chem., 2006, 78, 788-792.
12 A. Bhattacharyya and C. M. Klapperich, Microfluidics-based extraction of viral RNA from infected mammalian cells for disposable molecular diagnostics, Sens. Actuator B-Chem., 2008, 129, 693-698.
13 M. Mahalanabis, H. Al-Muayad, M. D. Kulinski, D. Altman and C. M. Klapperich, Cell lysis and DNA extraction of Gram-positive and Gram-negative Bacteria from Whole Blood in a Disposable Microfluidic Chip, Integrative Biology, 2009, submitted.
14 S. Gillers, C. D. Atkinson, C. Klapperich and S. K. Singh, Clostridium difficile Detection in Stool with a Nanoscale Microfluidic Device Gastroenterology, 2008, 134, A-578.
15 T. I. Wallow, A. M. Morales, B. A. Simmons, M. C. Hunter, K. L. Krafcik, L. A. Domeier, S. M. Sickafoose, K. D. Patel and A. Gardea, Low-distortion, high-strength bonding of thermoplastic microfluidic devices employing case-II diffusion-mediated permeant activation, Lab Chip, 2007, 7, 1825-1831.
16 V. Mengeaud, J. Josserand and H. H. Girault, Mixing processes in a zigzag microchannel: finite element simulations and optical study, Anal. Chem., 2002, 74, 4279-4286.

1. An integrated microfluidic device comprising a planar substrate having;
(i) at least one channel adapted to connect to a remote valve, wherein the remote valve can regulate and control the rate and direction of fluid flow on the microfluidic device; and
(ii) at least a first flow chamber and at least a second flow chamber, wherein the first flow chamber and the second flow chamber have different rates of flow

2. The integrated microfluidic device of claim 1 comprising at least one reservoir to serve as a fluid control buffer to control the flow rate and/or velocity of fluid flow between at least the first flow chamber and at least the second flow chamber on the planar substrate.

3. The integrated microfluidic device of claim 2, wherein the reservoir receives fluid from a first flow chamber.

4. The integrated microfluidic device of claim 2, wherein the reservoir receives fluid from a second flow chamber.

5. The integrated microfluidic device of claim 1, wherein the device comprises at least one reservoir which receives fluid from a first flow chamber, and at least one reservoir which receives fluid from a second flow chamber.

6. The integrated microfluidic device of claim 1, further comprising at least one output channel, where the output channel is adapted to connect to a remote valve, wherein the remote valve can regulate and control the direction of fluid flow on the microfluidic device.

7. The integrated microfluidic device of claim 1, further comprising at least one output channel fluidly connected to the reservoir, wherein the output channel is adapted to connect to a remote valve, wherein the remote valve can regulate and control the direction of fluid flow on the mircofluidic device.

8. The integrated microfluidic device of claim 7, further comprising PCR channels in fluid communication with at least one reservoir for thermal cycling of the fluid sample.

9. The integrated microfluidic device of claim 1, further comprising a sample detection well.

10. The integrated microfluidic device of claim 1, wherein the first flow chamber is a mixer.

11. The integrated microfluidic device of claim 1, wherein the second flow chamber is a SPE column.

12. The integrated microfluidic device of claim 1, wherein the substrate has low auto-fluorescence.

13. The integrated microfluidic device of claim 12, wherein the substrate is Zeonex®.

14. A system for controlling fluid flow on a microfluidic device of claim 1, the system comprising;
a. a microfluidic device comprising a planar substrate having;
i. at least one input channel adapted to connect to a remote valve, wherein the remote valve can regulate and control the rate and direction of fluid flow on the microfluidic device;
ii. at least one output channel adapted to connect to a remote valve, wherein the remote valve can regulate and control the direction of fluid flow on the microfluidic device; and
iii. at least one reservoir to serve as a fluid control buffer to control the flow rate of fluid flow between at least one chambers on the planar substrate;
b. at least one remote valve which is adapted to connect to at least one input channel on the microfluidic device;
c. at least one remote valve which is adapted to connect to at least one output channel on the microfluidic device; and
d. a control unit connected to each remote valve to control the opening and closing of the remote valves.

15. The system of claim 14, further comprising a thermal controller connected to the control unit, wherein the thermal controller controls the temperature of a thermal interface which interfaces with part of the microfluidic device.

16. The system of claim 14, further comprising a sample analysis detection system.

17. The system of claim 16, wherein the sample analysis detection system is connected to an optical interface which analyzes a sample present on the microfluidic device.

18. The system of any of claim 14, wherein a remote valve control fluid rate using any one of the following selected from the group of; pneumatic dispensers, syringe pumps, or flow restrictors.

19. The system of any of claim 14, wherein the microfluidic device is any of the microfluidic device according to claims 1 to 13.

20.-53. (canceled)

54. A remote-valve microfluidic device for controlling the flow of fluid in at least one channel on a microfluidic device; comprising;
a. a fluid-impermeable substrate, the substrate having at least one channel, the channel having an inlet on one external surface and an outlet at a different external surface,
b. a valve, the valve configured to be in an open position to allow fluid to pass through the channel from the inlet to the outlet or configured to be in a closed position to completely interrupt the flow of fluid from the inlet to the outlet,
wherein the external surface of the substrate with the inlet is fashioned for a reversible, fluidly sealed, engagement to an external surface of a second microfluidic device, creating an interface therebetween, and
wherein the channel of the substrate is capable of fluidly communicating with a channel on a second microfluidic device across the interface.

55. The remote-valve microfluidic device of claim 54, wherein the channel of second microfluidic has an inlet and an outlet, where the outlet is on the external surface of the second microfluidic device which forms the interface.

56. The remote-valve microfluidic device of claim 54, wherein the channel of second microfluidic has an inlet, at least one junction and at least two arm channels coming off the junction, each arm channel having an outlet on the same or different external surfaces of the second microchip.

57. The remote-valve microfluidic device of claim 54, optionally comprising a fluid impermeable material at the interface between the external surface at the second microfluidic device and the external surface of the substrate.

58. The remote-valve microfluidic device of claim 54, wherein the second microfluidic device is according to any of claims 1 to 13 or 34 to 49.

59-69. (canceled)

70. An apparatus, comprising;
a. at least one microfluidic device having at least one channel, said channel having at least one input and at least one output, wherein at least one input or at least one output are on the external surfaces of the microfluidic device;
b. a plurality of remote-valve switching devices, each containing a valve and at least one channel bias towards and reversibly sealed to, at least one microfluidic device, creating in interface therebetween; wherein the channel of each remote-valve switching device is capable of fluidly communicating with an input or output of a channel on the microfluidic device across the interface;
c. a means for opening and closing the valve in at least one remote-valve switching device, wherein an open valve position allows fluid to flow across the interface between the channel of the remove-valve switching device and the channel of the microfluidic device, whereas a closed valve position prevents the flow across the interface between the channel of the remove-valve switching device and the channel of the microfluidic device.

71. The apparatus of claim 70, wherein the microfluidic device is a valveless microfluidic device.

72. The apparatus of claim 70, wherein the microfluidic device is a microfluidic device of any of claims 1 to 13.

73. The integrated microfluidic device of claim 1, wherein the first flow chamber is a high flow chamber and the second flow chamber is a low flow chamber.

74. The integrated microfluidic device of claim 1, wherein the first flow chamber is a low flow chamber and the second flow chamber is a high flow chamber.
